(19) United States
(12) Patent Application Publication
(10) Pub. No.: US 2026/0355129 A1
(43) Pub. Date: Nov. 12, 2026

(54) 5-HT2C AGONISTS FOR TREATING PRADER-WILLI SYNDROME (PWS), METABOLIC SYNDROMES, AND OTHER DISEASES AND DISORDERS

(71) Applicant: Park City Bio, LLC, Salt Lake City, UT (US)

(72) Inventor: Jeremy Forest, Richmond Beach, WA (US)

(73) Assignee: Park City Bio, LLC, Salt Lake City, UT (US)

(21) Appl. No.: 19/636,592

(22) Filed: Apr. 1, 2026

Related U.S. Application Data
(63) Continuation of application No. PCT/US26/18754, filed on Mar. 11, 2026.
(60) Provisional application No. 63/770,584, filed on Mar. 12, 2025.
(60) Provisional application No. 63/805,185, filed on May 13, 2025.
(60) Provisional application No. 63/832,662, filed on Jun. 30, 2025.
(60) Provisional application No. 63/912,897, filed on Nov. 6, 2025.
(60) Provisional application No. 63/915,629, filed on Nov. 11, 2025.
(60) Provisional application No. 63/975,193, filed on Feb. 3, 2026.
(60) Provisional application No. 63/975,663, filed on Feb. 4, 2026.

(30) Foreign Application Priority Data
Apr. 1, 2025 (WO) PCT/US2025/022435

(51) Int. Cl. A61K 31/135; A61P 3/04; A61P 25/22

(52) U.S. Cl. CPC A61K 31/135; A61P 3/04; A61P 25/22

(57) ABSTRACT

This disclosure relates to the use of serotonin receptor agonists, optionally in combination with a GLP-1 agonist, for the treatment of metabolic disorders and other associated diseases that may be modulated via 5-HT_{2C} receptors.

### CROSS-REFERENCE TO RELATED APPLICATIONS

This application claims priority to U.S. Provisional Application No. 63/975,663, filed Feb. 4, 2026, U.S. Provisional Application No. 63/975,193, filed Feb. 3, 2026, U.S. Provisional Application No. 63/915,629, filed Nov. 11, 2025, U.S. Provisional Application No. 63/912,897, filed Nov. 6, 2025, U.S. Provisional Application No. 63/832,662, filed Jun. 30, 2025, U.S. Provisional Application No. 63/805,185, filed May 13, 2025, International Application No. PCT/US2025/022435, filed Apr. 1, 2025, and U.S. Provisional Application No. 63/770,584, filed Mar. 12, 2025, the entire contents of each are incorporated by reference herein for all purposes.

### TECHNICAL FIELD

The present disclosure relates to pharmaceutical compositions and methods for treating certain diseases and disorders including, but not limited to, metabolic disorders such as obesity, binge eating disorder, type 2 diabetes, and Prader-Willi Syndrome (PWS). In some embodiments, the present disclosure concerns the use of selective and/or biased 5-hydroxytryptamine 2C (5-HT_{2C}) receptor agonists and, optionally, a glucagon-like peptide-1 (GLP-1) receptor agonist.

### BACKGROUND

Prader-Willi syndrome (PWS) is a complex neurogenetic disorder in which hyperphagia and food-related compulsions frequently coexist with behavioral dysregulation, anxiety, and other neuropsychiatric symptoms. Current standards of care rely heavily on strict environmental control of food access, caloric supervision, and behavioral supports, with adjunctive pharmacologic management often directed to specific comorbidities rather than the core drive to eat. For example, recombinant human growth hormone is widely used in pediatric PWS to address growth failure and improve body composition, but it does not directly normalize hyperphagia or compulsive food-seeking. More recently, diazoxide choline extended-release (VYKAT™ XR) has been approved in the United States for treatment of hyperphagia in adults and pediatric patients (>4 years) with PWS, reflecting progress toward symptom-directed pharmacotherapy. Nevertheless, many patients continue to experience residual hyperphagia, compulsive behaviors, and related psychiatric burden, and commonly used off-label approaches can be limited by variable efficacy, tolerability, or lack of durable impact on the intertwined metabolic and behavioral features of PWS.

Accordingly, there remains a need for additional therapies that more effectively address PWS-associated symptomatology, including hyperphagia, compulsive behaviors, and downstream metabolic consequences, while maintaining an acceptable safety profile for chronic use across pediatric and adult populations. One possible approach is modulation of central serotonergic circuits governing appetite and impulse control, including the 5-HT_{2C} receptor pathway, which is mechanistically linked to melanocortin signaling and regulation of feeding behavior. The development of orally active, functionally biased 5-HT_{2C} agonists may provide a means to preferentially engage therapeutically beneficial signaling that suppresses appetite and improves behavioral control while minimizing signaling linked to dose-limiting adverse effects that have constrained prior serotonergic agents (e.g., the market withdrawal of the 5-HT_{2C} agonist lorcaserin). Such agents may therefore offer improved efficacy-tolerability balance and represent a potential next generation of targeted treatments for PWS and its associated compulsive and hyperphagic phenotypes. Such agents may also be useful in treating a variety of associated metabolic diseases and disorders, such as obesity and Type II diabetes, and may optionally be paired with other active agents like GLP-1 agonists to improve efficacy and patient outcomes.

Accordingly, there remains a need to develop biased 5-HT_{2C} receptor agonists for the treatment of metabolic diseases and disorders such as PWS.

### SUMMARY

The present disclosure provides compositions and methods for treating certain diseases and disorders, including metabolic disorders, comprising administering to a subject in need thereof at least one 5-HT_{2C} receptor agonist.

In certain embodiments, the present disclosure provides compositions and methods for treating Prader-Willi Syndrome (PWS) comprising administering to a subject in need thereof at least one 5-HT_{2C} receptor agonist. In certain embodiments, the at least one 5-HT_{2C} receptor agonist is functionally biased.

The present disclosure also provides compositions and methods for treating metabolic disorders using a combination of:
a 5-HT_{2C} receptor agonist; and
a GLP-1 receptor agonist.

In some embodiments, the 5-HT_{2C} agonist is functionally biased. In some embodiments, the 5-HT_{2C} agonist is administered orally. In some embodiments, the 5-HT_{2C} agonist exhibits minimal activity at the 5-HT_{2A} and/or 5-HT_{2B} receptor subtypes. In further embodiments, the 5-HT_{2C} agonist has an oral half-life in humans of at least 5 hrs and is administered at least once daily.

In some embodiments, the GLP-1 receptor agonist may be selected from liraglutide, semaglutide, dulaglutide, albiglutide, tirzepatide, and other analogs. In some embodiments, the GLP-1 agonist may be administered weekly or monthly by injection, or daily in oral form (e.g., semaglutide/Rybelsus®).

In certain embodiments, the agents are co-administered, either as separate dosage forms or in a single kit with combined instructions. In some embodiments, the combination is indicated for patients with suboptimal response to GLP-1 receptor agonist therapy alone.

Unless stated otherwise, general reference to Formula I includes all compounds encompassed by Formula I, including those selected from subgenera Formula II, Formula II(a), Formula II(b), Formula V, Formula VI(a), etc.

### BRIEF DESCRIPTION OF THE DRAWINGS

FIG. 1A provides the graphical results of active lever responses in the rodent operant self-administration compulsivity study described in the Examples section of this application.

FIG. 1B provides the graphical results of ethanol intake in the rodent operant self-administration compulsivity study described in the Examples section of this application.

### DETAILED DESCRIPTION

### Methods

The present disclosure provides compositions and methods for treating diseases and disorders, including metabolic disorders, comprising administering to a subject in need thereof at least one 5-HT_{2C} receptor agonist. In certain embodiments, the at least one 5-HT_{2C} receptor agonist is biased.

The present disclosure further provides compositions and methods for treating Prader-Willi Syndrome (PWS), as well as symptoms associated therewith, comprising administering to a subject in need thereof at least one 5-HT_{2C} receptor agonist.

The present disclosure further provides compositions and methods for treating metabolic disorders using a combination of:
a 5-HT_{2C} receptor agonist; and
a GLP-1 receptor agonist.

In some embodiments, the 5-HT_{2C} agonist is functionally biased. As used herein, the term “biased” or “functionally biased” refers to the ability of a ligand to selectively activate specific intracellular signaling pathways downstream of the receptor, rather than uniformly activating all possible signaling cascades. For 5-HT_{2C} receptors, this often involves preferential stimulation of the Gq/PLC/IP₃/Ca²⁺ pathway over alternative pathways such as β-arrestin recruitment or other G-protein subtypes. For example, certain known 5-HT_{2C} receptor agonists such as lorcaserin are non-biased in that they signal strongly through both the Gq and β-arrestin signaling pathways. Conversely, compounds described herein, such as those of Formula I and II, are biased and preferentially signal through Gq over β-arrestin at the 5-HT_{2C} receptors. Thus, in certain embodiments, the 5-HT_{2C} receptor agonist is functionally biased and exhibits a β-arrestin recruitment efficacy (Emax) of less than about 50% that of serotonin in an in vitro assay (e.g., 5-HT_{2C} BRET assay). Biased agonists can enhance therapeutic efficacy (e.g., appetite suppression, impulse control) while minimizing undesirable side effects, such as anxiety or cardiovascular effects that may result from off-target or full-spectrum receptor activation. In certain embodiments, this selectivity is particularly valuable at 5-HT_{2C} receptors, where finely tuned signaling can improve safety and tolerability relative to non-biased or less selective compounds.

Exemplary metabolic disorders for treatment using the methods and protocols described herein include, but are not limited to, obesity, binge eating disorders, Type 2 Diabetes, metabolic syndrome, non-alcoholic fatty liver disease, non-alcoholic steatohepatitis, prediabetes, insulin resistance, polycystic ovary syndrome, hypothalamic obesity, dyslipidemia/hyperlipidemia, hyperinsulinemia/hyperglycemia, Prader-Willi Syndrome, alcohol use disorder, drug use disorders (e.g., nicotine, cocaine, methamphetamine), anorexia nervosa, and sleep apnea.

In some embodiments is described a method of treating Prader-Willi Syndrome comprising administering at least one compound of Formula I to a subject in need thereof. In certain embodiments is described a method of treating at least one symptom associated with Prader-Willi Syndrome comprising administering at least one compound of Formula I to a subject in need thereof.

Prader-Willi syndrome (PWS) is a genetic disease caused by lack of expression of genes from an imprinted region of the paternally inherited chromosome 15qI I-qI3, near the centromere. The frequency of the disease is between about 1/10,000 and 1/30,000 with approximately 400,000 PWS patients living worldwide. PWS is a spectrum disorder which affects many systems in the body. Subjects with PWS typically suffer from a host of symptoms including neurologic, cognitive, endocrine, and behavioral abnormalities. Initially, infants exhibit hypotonia (floppy baby syndrome) and experience difficulty in sucking and feeding which can lead to growth delay. Subjects with PWS frequently have poor muscle tone, growth hormone deficiency, low levels of sex hormones, a constant feeling of hunger and excessive appetite (hyperphagia). They overeat, leading to weight gain, obesity and a high incidence of diabetes. Other signs appear including short stature, poor motor skills, underdeveloped sex organs, and mild intellectual and learning disabilities. PWS subjects may experience delayed speech and language development, and infertility. Behavioral symptoms may include cognitive impairment, cognitive rigidity, emotional lability and obsessive-compulsive behavior, with autistic symptomology, psychotic episodes, and bipolar disorder with psychosis. Additional clinical manifestations may include excessive daytime sleepiness, scoliosis, osteopenia/osteoporosis, decreased gastrointestinal motility, sleep disturbances, and reduced pain sensitivity.

In some embodiments is described a method of improving at least one symptom associated with Prader-Willi Syndrome comprising administering an effective amount of at least one compound of Formula I to a subject in need thereof, wherein the at least one symptom is selected from hypotonia, difficulty in sucking, difficulty in feeding, poor muscle tone, growth hormone deficiency, low levels of sex hormones, a constant feeling of hunger, excessive appetite (hyperphagia), weight gain, obesity, short stature, poor motor skills, underdeveloped sex organs, intellectual disability, learning disability, delayed speech development, delayed language development, infertility, cognitive impairment, cognitive rigidity, emotional lability, self-injury, obsessive-compulsive behavior, autistic symptomology, psychotic episodes, bipolar disorder with psychosis, excessive daytime sleepiness, scoliosis, osteopenia/osteoporosis, decreased gastrointestinal motility, sleep disturbances, and/or reduced pain sensitivity.

In certain embodiments is described a method of treating at least one symptom associated with Prader-Willi Syndrome (e.g., effectively treating two or more symptoms) comprising administering an effective amount of at least one compound of Formula I to a subject in need thereof, wherein the treatment of the at least one symptom is selected from: reducing or eliminating difficulty in sucking, reducing or eliminating difficulty in feeding, reducing or eliminating poor muscle tone, reducing or eliminating growth hormone deficiency, increasing levels of sex hormones, reducing or eliminating a constant feeling of hunger, reducing or eliminating excessive appetite (hyperphagia), reducing or eliminating weight gain, reducing or eliminating obesity, reducing or eliminating short stature, increasing motor skills, reducing or eliminating underdeveloped sex organs, reducing or eliminating intellectual disability, reducing or eliminating learning disability, reducing or eliminating delayed speech development, reducing or eliminating delayed language development, reducing or eliminating infertility, reducing or eliminating cognitive rigidity, reducing or eliminating cognitive impairment, reducing or eliminating emotional lability, reducing or eliminating obsessive-compulsive behavior, reducing or eliminating aggression, reducing or eliminating a compulsive behavior (e.g., drug or alcohol use), reducing or eliminating hyperactivity, reducing or eliminating cognitive impairment (e.g., improving memory), reducing or eliminating bipolar disorder with psychosis, reducing or eliminating excessive daytime sleepiness, reducing or eliminating scoliosis, reducing or eliminating osteopenia/osteoporosis, reducing or eliminating decreased gastrointestinal motility, reducing or eliminating sleep disturbances (e.g., including disturbances in REM sleep), reducing or eliminating stress or anxiety, reducing or eliminating reduced pain sensitivity, enhancing cognitive function, increasing daytime activity, improving learning (either the rate or ease of learning), improving attention, improving social behavior, and/or improving cerebrovascular function.

In embodiments, effective amount refers to an amount which may be suitable to prevent a decline in any one or more of the above qualities, or, in embodiments, to improve any one or more of the above qualities, for example, constant feeling of hunger, excessive appetite (hyperphagia), weight gain, obesity, cognitive function or performance, learning rate or ability, problem solving ability, attention span and ability to focus on a task or problem, social behavior, and the like. In some embodiments, an effective amount may be suitable to reduce either the extent or rate of decline in a subject's appetite dysregulation, weight loss, cognitive skills and/or functioning, and/or the effective amount may be suitable to delay the onset of such decline. In embodiments, an effective amount increases hypothalamic brain-derived neurotrophic factor (BDNF) expression. Such effectiveness may be achieved, for example, by administering the compounds and/or compositions described herein to an individual or to a population. In some embodiments, the reduction, or delay of such a decline, or the improvement in an individual or population can be relative to a cohort, e.g., a control subject or a cohort population that has not received the treatment or been administered the composition or medicament.

In some embodiments, the 5-HT_{2C} agonist is administered orally. In some embodiments, the 5-HT_{2C} agonist is administered via injection. In some embodiments, the 5-HT_{2C} agonist is administered intravenously (IV). In some embodiments, the 5-HT_{2C} agonist is administered subcutaneously (SC).

In one embodiment is described a method of treating a metabolic disorder, comprising administering to a subject in need thereof:
at least one 5-HT_{2C} receptor agonist; and
a GLP-1 receptor agonist,
   wherein the 5-HT_{2C} receptor agonist is administered at least once every other day, and the GLP-1 receptor agonist is administered at least once per month. In some embodiments, the 5-HT_{2C} receptor agonist is administered orally. In some embodiments, the GLP-1 agonist is administered by injection (e.g., subcutaneously).

In some embodiments, the GLP-1 receptor agonist is administered orally. In some embodiments, the GLP-1 receptor agonist is administered via injection. In some embodiments, the GLP-1 receptor agonist is administered intravenously (IV). In some embodiments, the GLP-1 receptor agonist is administered subcutaneously (SC). In some embodiments, the agonists are administered in therapeutically effective amounts.

In one embodiment is described a method of treating a metabolic disorder, comprising providing instructions to a patient or a healthcare provider to:
administer to a subject in need thereof at least one 5-HT_{2C} receptor agonist; and
administer to a subject in need thereof a GLP-1 receptor agonist,
   wherein the instructions indicate that the combination therapy improves weight loss outcomes relative to monotherapy.

In one embodiment is described a method of inducing weight loss in a subject (such as an overweight or obese subject) comprising administering to a subject in need thereof:
a 5-HT_{2C} receptor agonist having a half-life of at least 5 hours; and
a GLP-1 receptor agonist having a half-life of at least 72 hours,
   wherein the subject achieves at least a 5% reduction in body weight after 12 weeks of therapy.

In certain embodiments, the subject receiving the combination therapy achieves at least about a 5% reduction in body weight after 12 weeks of therapy, such as at least 8% or at least 10% reduction in body weight. In certain embodiments, the subject achieves a reduction of about 5% to about 12% after 12 weeks of therapy. In certain embodiments, the subject achieves a reduction of about 8% to about 15% after 12 weeks of therapy. As used herein, “body weight” refers to the total mass of a subject, measured in kilograms (kg) or grams (g), typically assessed using a calibrated scale. For non-human animal models, body weight is measured at baseline (Day 0) and at one or more subsequent timepoints during the study period. For human subjects, body weight refers to the total physical mass of the individual and is used as a primary indicator of obesity, metabolic status, or therapeutic response. Unless otherwise specified, percent weight loss refers to the percentage change in body weight from baseline (pre-treatment) to a specified post-treatment timepoint.

In some embodiments, therapeutic efficacy is evaluated based on percent reduction in body weight or body mass index (BMI). In human subjects, a clinically meaningful response may correspond to a reduction in body weight of at least 5%, or a reduction in BMI of at least 2 units or 5% from baseline. Accordingly, for any of the embodiments described herein wherein body mass (or changes in body mass) is calculated, body mass may be replaced by BMI. For example, where “reduction in body weight after 12 weeks of therapy” in some embodiments may be replaced with “reduction in BMI after 12 weeks of therapy.”

Some research has demonstrated that GLP-1 receptor agonists (like semaglutide and tirzepatide) cause substantial total weight loss, but a disproportionate share can come from lean body mass (LBM), including skeletal muscle. Indeed, some studies have shown that 2.4 mg dosing of semaglutide over 68 weeks can result in a total average weight loss of about 15%, wherein 39% of that total weight loss is lean body mass loss (about 6% of initial lean body mass). Similar studies with tirzepatide have shown about 25-40% of total weight loss can be attributed to a loss in lean mass, accounting for a 5-7% loss in LBM over 72 weeks.

Contrariwise, in some embodiments, Applicant has surprisingly discovered that therapies described herein can result in a loss of lean body mass of no more than 3% baseline LBM, or the loss of LBM accounts for no more than 25% of total weight loss, indicating preservation of active tissue. In certain embodiments, the methods described herein result in preservation of at least 75% of the subject's lean body mass during weight loss. As used herein unless stated differently, lean body mass (LBM) refers to the total weight of a person's body excluding fat mass. It includes skeletal muscle, bone, organs, skin, connective tissue, body water, and other fat-free components.

In certain embodiments, the 5-HT_{2C} receptor agonist is functionally biased. In certain embodiments, the 5-HT_{2C} receptor agonist is administered orally. In certain embodiments, the 5-HT_{2C} receptor agonist exhibits an oral half-life of at least 5 hours (hrs) in humans, such as about 5 hrs to about 24 hrs or about 6 hrs to about 15 hrs. In certain embodiments, the 5-HT_{2C} receptor agonist has a half-life of about 3 hrs to about 6 hrs in a mouse model following oral administration. In certain embodiments, the GLP-1 agonist is administered orally. In certain embodiments, the GLP-1 receptor agonist is administered by injection. In some embodiments, the GLP-1 agonist exhibits a subcutaneous half-life of at least 72 hours in humans, such as about 72 hrs to about two weeks.

In one embodiment is described a method of treating obesity in a subject in need thereof, comprising:
initiating treatment with a GLP-1 receptor agonist administered at least once per month; and
initiating co-treatment with a selective 5-HT_{2C} receptor agonist at least once every other day if weight loss in the subject is suboptimal after 4-8 weeks of GLP-1 receptor agonist monotherapy.
   In certain embodiments, the co-treatment results in a statistically significant increase in mean weight loss compared to continued GLP-1 receptor agonist monotherapy.

In certain embodiments, the suboptimal weight loss after 4-8 weeks of GLP-1 receptor agonist monotherapy is less than an 8% reduction of body weight. In certain embodiments, the suboptimal weight loss after 4-8 weeks of GLP-1 receptor agonist monotherapy is less than a 5% reduction of body weight, such as a reduction of about 1% to about 4% body weight.

In one embodiment is described a method of treating a metabolic disorder, comprising the simultaneous, sequential, or separate administration to a subject in need thereof of:
a 5-hydroxytryptamine 2C (5-HT_{2C}) receptor agonist; and
a glucagon-like peptide-1 (GLP-1) receptor agonist.

In certain embodiments, the 5-HT_{2C} receptor agonist is functionally biased and/or exhibits at least 100-fold selectivity for the 5-HT_{2C} receptor over the 5-HT_{2A} and 5-HT_{2B} receptors. In certain embodiments, the metabolic disorder is obesity, binge eating disorder, Type 2 diabetes, or a combination thereof. In certain embodiments, the combination results in a greater reduction in body weight or binge eating behavior than that achieved by either agent alone.

In one embodiment is described a method of treating a metabolic disorder comprising:
administering to a patient a combination of a GLP-1 receptor agonist and a 5-HT_{2C} receptor agonist, wherein the administration of the combination is conducted simultaneously, sequentially or separately, and said administration achieves a desired outcome, wherein the patient has previously been administered a GLP-1 receptor agonist treatment regimen for treating the metabolic disorder, and the treatment regimen did not achieve the desired outcome.

In certain embodiments, the desired outcome may be: at least a 5% reduction in body weight after 12 weeks of the combination therapy, such as at least 8% or at least 10% reduction in body weight; a reduction of about 8% to about 15% after 12 weeks of the combination therapy; a statistically significant increase in mean weight loss compared to continued GLP-1 receptor agonist monotherapy; or improvement in weight loss outcomes relative to GLP-1 receptor agonist monotherapy.

In one embodiment is described a kit comprising:
a first container comprising an oral dosage form of a 5-HT_{2C} receptor agonist;
a second container comprising an injectable dosage form of a GLP-1 receptor agonist; and
printed instructions for concomitant use of the selective 5-HT_{2C} receptor agonist and the GLP-1 receptor agonist for the treatment of obesity. In some embodiments, the printed instructions can include language printed on paper or another medium within the kit. In some embodiments, the printed instructions can be accessed by a barcode or QR code contained in the kit, wherein scanning of said code with a smartphone or other device provides the instructions in digital form.

In one embodiment is described a method of treating binge eating disorder in a human subject comprising concomitantly administering to the subject:
a therapeutically effective amount of a 5-HT_{2C} receptor agonist at least once every other day; and
a therapeutically effective amount of a GLP-1 receptor agonist at least once per month.

In some embodiments, the treatment results in a reduction in binge eating episodes and/or overall weight loss.

In one embodiment is described a system for promoting weight loss in a subject comprising:
a regimen of administration of a 5-HT_{2C} receptor agonist and a GLP-1 receptor agonist; and
a software application configured to track at least one of medication adherence, monitor weight and/or appetite changes, and/or to adjust dosage recommendations based on subject response.

In one embodiment is described a method of treating obesity in a human subject comprising:
(a) administering to the subject a 5-HT_{2C} receptor agonist at least once every other day; and
(b) administering to the subject a glucagon-like peptide-1 (GLP-1) receptor agonist at least once per month.

In some embodiments, the combination of (a) and (b) provides for a synergistic effect characterized by a reduction in body weight that is greater than the sum of the reductions observed when each agonist is administered as a monotherapy.

In one embodiment is described a method of treating obesity or a binge eating disorder in a subject, comprising administering a functionally biased 5-HT_{2C} receptor agonist and a GLP-1 agonist, wherein the 5-HT_{2C} receptor agonist selectively activates a Gq-mediated signaling pathway with reduced recruitment of β-arrestin relative to a non-biased 5-HT_{2C} receptor agonist, and wherein administration of the biased agonist results in a durable therapeutic response, characterized by a sustained reduction in body weight or binge-eating episodes over a period of at least 8 weeks, and wherein the durable therapeutic response is further characterized by exceeding the duration of response observed with an equi-effective dose of a non-biased 5-HT₂C receptor agonist administered under otherwise equivalent conditions.

In certain embodiments, the GLP-1 agonist comprises an oral dosage form selected from semaglutide (Rybelsus®), orforglipron (investigational), and danuglipron (investigational). In certain embodiments, the GLP-1 agonist comprises an injectable dosage form selected from semaglutide (Ozempic, Wegovy), liraglutide (Victoza, Saxenda), dulaglutide (Trulicity), exenatide (Byetta, Bydureon), albiglutide (Tanzeum—withdrawn), afpeglenatide (investigational), and tirzepatide (Mounjaro, Zepbound—dual GIP/GLP-1 agonist).

In some embodiments, the subject experiences 15% reduction in baseline body weight after 12 weeks of combination therapy.

In one embodiment, the synergistic effect of the combination therapy is observed in preclinical rodent models fed a high-fat diet, as measured by percent reduction in body weight relative to baseline. In one embodiment, the synergistic effect is evidenced by a statistically significant increase (p<0.05) in mean percent weight loss at 12 weeks in subjects receiving the combination compared to the expected additive weight loss based on matched monotherapy groups.

In certain embodiments, the subject/patient is a mammal. In certain embodiments, the subject is a primate. In certain embodiments, the subject is a non-human primate. In certain embodiments, the subject is a human.

In certain embodiments, the 5-HT_{2C} agonist is functionally biased. In certain embodiments, the 5-HT_{2C} agonist is selective, wherein “selective” means at least 100-fold selectivity over 5-HT_{2A} and/or 5-HT_{2B} receptors as measured in human cells via one or more functional assays for serotonin receptors (e.g., BRET assays).

Unless context dictates otherwise, the term “at least once every other day” means a patient is administered a minimum treatment of one or more times every other day and, thus, would include, e.g., once per day, 2-3 times per day, and once every other day regimens.

Unless context dictates otherwise, the term “at least once per month” means a patient is administered a minimum treatment of one time every month and, thus, would include, e.g., once per month, once per week, once per day, and once every other day regimens.

In some embodiments, the 5-HT_{2C} agonist exhibits minimal activity at the 5-HT_{2A} and/or 5-HT_{2B} receptor subtypes. In further embodiments, the 5-HT_{2C} agonist has an oral half-life in humans of at least 5 hrs and is administered at least once daily. In some embodiments, the 5-HT_{2C} agonist has an oral half-life in humans of at least 8 hrs and is administered at least once daily. In some embodiments, the 5-HT_{2C} agonist has an oral half-life in humans of at least 12 hrs and is administered at least once daily. In some embodiments, the at least one 5-HT_{2C} receptor agonist is selected from compounds of Formula I.

In some embodiments, the 5-HT_{2C} agonists described herein may be useful in the treatment of other diseases or disorders that may be modulated via 5-HT_{2C} agonism. Such diseases and disorders may include, but are not limited to, a broad range of psychological, neurological, developmental, inflammatory, and pain-related conditions. The psychological and psychiatric disorders may include, but are not limited to, depression; psychotic disorders; schizophrenia; schizophreniform disorder; schizoaffective disorder; bipolar I disorder (mania/manic-depressive psychosis); bipolar II disorder; major depressive disorder (including with psychotic features); delusional disorders (paranoia); shared psychotic disorder; brief psychotic disorder; unspecified psychosis; paranoid, schizoid, and schizotypal personality disorders; anxiety disorders (including social anxiety disorder, substance-induced anxiety disorder, panic disorder, panic attacks, agoraphobia, and selective mutism); attention deficit syndrome; post-traumatic stress disorder (PTSD); premenstrual dysphoric disorder (PMDD); and premenstrual syndrome (PMS).

In certain embodiments, the disease or disorder includes neurodegenerative disorders, including Huntington's disease, Alzheimer's disease, dementia, and Parkinson's disease, as well as developmental disorders; delirium; amnestic and other cognitive disorders; psychiatric disorders due to a somatic condition; drug-related disorders; mood disorders; somatoform disorders; factitious disorders; dissociative disorders; eating disorders; sleep disorders; impulse control disorders; adjustment disorders; and personality disorders.

In addition, the disclosure includes the treatment or prevention of inflammation and pain, including inflammatory skeletal or muscular conditions, joint pain, muscle pain, tendon pain, burn pain, and pain associated with inflammatory diseases such as rheumatoid arthritis, and encompasses nociceptive, neuropathic, mixed-type, acute, and chronic pain. Other inflammatory disorders may include Lyme Disease and Post-Treatment Lyme Disease (PTLD).

In certain embodiments, the present disclosure provides methods for treating dysautonomia and related symptomatology associated with Lyme disease, including post-treatment Lyme disease (PTLD), by administering to a subject in need thereof a therapeutically effective amount of one or more 5-HT_{2C} receptor agonist described herein. Dysautonomia in the context of Lyme disease or PTLD may manifest as orthostatic intolerance, postural tachycardia, gastrointestinal dysmotility, altered thermoregulation, palpitations, exercise intolerance, fatigue, sleep disturbance, cognitive impairment (“brain fog”), anxiety, depression, anhedonia, and other neuropsychiatric or autonomic symptoms, many of which are believed to be driven or exacerbated by persistent neuroinflammation, peripheral inflammation, endothelial dysfunction, or immune dysregulation. Without being bound by theory, it is believed that activation of 5-HT_{2C} receptors within central autonomic regulatory nuclei, including hypothalamic and brainstem circuits, modulates sympathetic and parasympathetic tone, regulates hypothalamic-pituitary-adrenal (HPA) axis activity, and/or attenuates aberrant neuroimmune signaling, including reductions in pro-inflammatory cytokines and microglial activation. Such effects may restore autonomic balance, improve stress responsivity, and alleviate both somatic and neurobehavioral symptoms associated with inflammatory or post-infectious states.

In further embodiments, the methods described herein are applicable to additional disorders that share overlapping symptom domains and pathophysiologic features with Lyme disease and PTLD, including post-viral syndromes and other chronic inflammatory conditions. Non-limiting examples include post-acute sequelae of viral infection (e.g., post-acute sequelae of SARS-CoV-2 infection (PASC or “long COVID”)), myalgic encephalomyelitis/chronic fatigue syndrome (ME/CFS), postural orthostatic tachycardia syndrome (POTS), fibromyalgia, mast cell activation-associated disorders, and other conditions characterized by dysautonomia, central sensitization, chronic fatigue, cognitive dysfunction, mood disturbance, and/or systemic or neuroinflammation. In such disorders, selective and/or functionally biased 5-HT_{2C} agonists, including orally bioavailable small molecules such as compounds of Formula I described herein, may be administered alone or in combination with antiviral, anti-inflammatory, immunomodulatory, or supportive therapies to reduce inflammatory burden, normalize autonomic signaling, improve fatigue and cognitive performance, and ameliorate associated psychiatric and somatic symptoms.

In addition, the 5-HT_{2C} agonists described herein may be used to treat a variety of seizure disorders, including both convulsive and non-convulsive epilepsies. Exemplary seizure types include absence seizures (staring spells and/or loss of awareness), such as Childhood Absence Epilepsy (CAE) and Juvenile Absence Epilepsy (JAE), including forms with or without myoclonia and Jeavons Syndrome (absence seizures with eyelid myoclonia). Absence seizures are characterized by brief episodes of loss of awareness or behavioral arrest, often lasting only seconds and occurring multiple times per day and are described as non-convulsive because they do not involve rhythmic jerking or sustained muscle stiffening. Seizure disorders may also include generalized convulsive seizure types, including tonic-clonic seizures (body stiffening followed by jerking movements), clonic seizures (repetitive rhythmic jerking), myoclonic seizures (sudden, brief muscle twitches), and atonic seizures (sudden loss of muscle tone resulting in falls). In addition, focal (partial) seizures are included, which may present with region-specific symptoms depending on the cortical area involved, such as unusual sensory experiences or involuntary movements in a localized body region.

In certain embodiments, the compounds described herein may be effective across mechanistically distinct seizure categories-both convulsive and non-convulsive-despite the historical divergence in pathophysiology and treatment response between these classes. In certain embodiments, the compounds may be used to treat mixed seizure phenotypes, such as juvenile absence epilepsy with occasional convulsive episodes, who otherwise often require multiple agents to address different seizure types. Additionally, certain embodiments contemplate seizure disorders associated with genetic dysfunction, including mutations in ion channel and synaptic genes such as SCN1A, SCN2A, SCN8A, CACNA1A, CACNA1H, GABRG2, GABRB3, and others. Other embodiments include methods of increasing serotonin and its metabolites in the brainstem by administering one or more functionally biased 5-HT_{2C} agonists, including those compounds described herein. In certain embodiments, the present disclosure provides novel 5-HT_{2C} receptor agonists that exhibit unique in vivo pharmacodynamic properties. Notably, in some embodiments, administration of the disclosed compounds results in elevated concentrations of serotonin (5-HT) and its primary metabolite 5-hydroxyindoleacetic acid (5-HIAA) in brainstem regions, including but not limited to the dorsal raphe nucleus (DRN). This neurochemical profile is surprising and unexpected based on the established mechanistic role of 5-HT_{2C} receptors in serotonergic circuits.

Prior to the work conducted herein, activation of 5-HT_{2C} receptors has been widely reported to inhibit serotonergic neuron firing in the dorsal raphe nucleus through activation of local GABAergic interneurons. Consistent with this mechanism, pharmacological activation of 5-HT_{2C} receptors with agonists such as RO-60-0175 has been shown to suppress increases in serotonin turnover under stress conditions, as reflected by reductions in the 5-HIAA/5-HT ratio (see Mongeau et al., J. Neurochem., 2010, 115(2):438-49). In view of this established inhibitory role of 5-HT_{2C} signaling on serotonergic activity, an observed increase in both serotonin (5-HT) and its principal metabolite 5-HIAA within brainstem regions following administration of a selective 5-HT_{2C} agonist would not have been predicted based on the prevailing pharmacological understanding of 5-HT_{2C} receptor function.

In contrast, administration of certain compounds of the present disclosure can produce statistically significant increases in 5-HT and 5-HIAA concentrations within the brainstem, suggesting either a novel mechanism of receptor signaling (e.g., biased agonism), region-specific effects on serotonin biosynthesis or metabolism, and/or alternative modulation of presynaptic serotonergic regulation. This outcome is unexpected in view of the canonical understanding of 5-HT_{2C} pharmacology and would not have been predicted by prior art. This makes the compounds disclosed herein of particular interest, as they seem to modulate a mechanism of action that is unique from those modulated by previously-identified 5-HT_{2C} agonists.

In some embodiments, the compounds and compositions described herein may be suitable for treating, preventing or ameliorating the effects of a variety of other seizure-related disorders, such as sudden unexpected death in epilepsy (SUDEP), atonic (aka “drop”) seizures, and audiogenic seizures. As used herein, SUDEP generally refers to refers to the sudden, unexpected death of someone with epilepsy, who was otherwise healthy and for whom no other cause of death is found during autopsy. SUDEP is one of the leading causes of death in people with uncontrolled seizures, particularly generalized tonic-clonic seizures. In certain embodiments, it is believed that SUDEP may be associated with cardiac arrhythmias triggered by post-ictal autonomic dysfunction, respiratory suppression following seizures (central apnea), or brainstem dysfunction (potentially similar to mechanisms seen in audiogenic seizures in animal models).

### Compounds

In certain embodiments, the 5-HT_{2C} agonist is functionally biased. In some embodiments, the 5-HT_{2C} agonist is selected from compounds of Formula I:

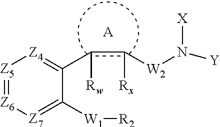

wherein
X and Y are each independently selected from hydrogen, deuterium, optionally substituted C₁-C₈ alkyl, optionally substituted C₂-C₈ alkenyl, and optionally substituted C₂-C₈ alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from CF₂, NR₁, O, S, S(O), SO₂, Se, Se(O), and SeO₂;
W₂ is absent or C(R₃)(R_{3′});
Z₄ is selected from N and CR₄;
Z₅ is selected from N and CR₅;
Z₆ is selected from N and CR₆;
Z₇ is selected from N and CR₇;
R₁ is selected from hydrogen, deuterium, optionally substituted C₁-C₈ alkyl, optionally substituted C₂-C₈ alkenyl, —C(O)R₃, —C(O)OR₃, —P(O)(OR₉)₂, —C(O)N(R₉)₂, —SOR₃, and —SO₂R₈;
R₂, R₄, R₅, R₆, and R₇ are each independently selected from hydrogen, deuterium, —N(R₉)₂, —SR₉, halo, optionally substituted C₁-C₈ alkyl, —C₁-C₈ alkoxy, optionally substituted C₂-C₈ alkenyl, optionally substituted C₂-C₈ alkynyl, optionally substituted aryl, optionally substituted heteroaryl, hydroxyl, —OC(O)R₈, —OC(O)OR₈, —OP(O)(OR₉)₂, and —OSO₂R₈, or R₂ is a residue selected from Formula III:

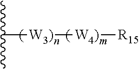

wherein W₃ is, for each occurrence, independently selected from optionally substituted alkylene, optionally substituted arylene, optionally substituted heteroarylene, optionally substituted cycloalkylene, optionally substituted heterocycloalkylene, and

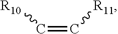

wherein n is an integer selected from 1 to 10,
W₄ is, for each occurrence, independently selected from O, S, Se, C(R₁₂)(R₁₃), optionally substituted arylene, optionally substituted heteroarylene, optionally substituted cycloalkylene, optionally substituted heterocycloalkylene, and

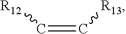

wherein m is an integer selected from 0 to 10, and
R₁₀, R₁₁, R₁₂, R₁₃, and R₁₅ are, for each occurrence, independently selected from optionally substituted C₁-C₈ alkyl, hydrogen, deuterium, halo, and hydroxyl,
or R₄ is taken together with R₅ and the carbons therebetween to form 3- to 7-membered carbocyclic or heterocyclic ring optionally including 1 to 2 additional ring heteromoieties selected from O, S, S(O), SO₂, and NR₉, or R₅ is taken together with R₆ and the carbons therebetween to form 3- to 7-membered carbocyclic or heterocyclic ring optionally including 1 to 2 additional ring heteromoieties selected from O, S, S(O), SO₂, and NR₉, or R₆ is taken together with R₇ and the carbons therebetween to form 3- to 7-membered carbocyclic or heterocyclic ring optionally including 1 to 2 additional ring heteromoieties selected from O, S, S(O), SO₂, and NR₉;
R₃ and R_{3′} are each, independently, selected from hydrogen, deuterium, —N(R₉)₂, —SR₉, halo, optionally substituted C₁-C₈ alkyl, −C₁-C₈ alkoxy, and optionally substituted C₂-C₈ alkenyl, or Y is absent and R₃ taken together with carbon to which it is attached and the nitrogen atom to which X is attached form a 3- to 7-membered heterocyclic ring optionally including 1 to 2 additional ring heteromoieties selected from O, S, S(O), SO₂, and NR₉, or R₃ and R_{3′} are taken together with the carbon to which they are attached to form a keto group;
Rₓ and R_{w} are absent, or each, independently, is selected from hydrogen, deuterium, —N(R₉)₂, —SR₉, halo, optionally substituted C₁-C₈ alkyl, −C₁-C₈ alkoxy, optionally substituted C₂-C₈ alkenyl, and optionally substituted C₂-C₈ alkynyl, or R₂ is taken together with W₁ and the carbon to with Rₓ is attached, and the carbons therebetween, to form a 3- to 7-membered carbocyclic or heterocyclic ring optionally including 1 to 2 additional ring heteromoieties selected from O, S, S(O), SO₂, and NR₉;
R₈ is selected from optionally substituted C₁-C₈ alkyl, optionally substituted C₂-C₈ alkenyl, and optionally substituted aryl;
R₉ is independently selected from hydrogen, deuterium, optionally substituted C₁-C₈ alkyl, optionally substituted C₂-C₈ alkenyl, and optionally substituted aryl;

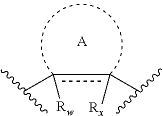

(also referred to as “Group A”) represents a fused ring (in other words, a ring system fused to the rest of the molecular scaffold) chosen from an optionally substituted cycloalkanyl, optionally substituted cycloalkenyl, optionally substituted heterocyclyl, optionally substituted aryl, and optionally substituted heteroaryl;
the [symbol] is a single or double bond, wherein Rₓ and R_{w} are absent when it is a double bond;
and salts, solvates, hydrates, prodrugs, and enantiomers thereof.

As used herein, the term “alkyl” refers to straight, branched or cyclic saturated hydrocarbon group. As used herein, alkyl has 1 to 20 carbon atoms, 1 to 10 carbon atoms, 1 to 8 carbon atoms, 1 to 6 carbon atoms or 1 to 3 carbon atoms. Examples of alkyl groups include methyl, ethyl, propyl, isopropyl, n-butyl, isobutyl, sec-butyl, tert-butyl pentyl, isopentyl, hexyl, heptyl, octyl, as well as cycloalkyl variants such as cyclopropyl, cyclobutyl, cyclopentyl, cyclohexyl, cycloheptyl, cyclooctyl, cyclononyl, and cyclodecyl. When an alkyl residue having a specific number of carbons is named by chemical name or identified by molecular formula, all positional isomers having that number of carbons may be encompassed; thus, for example, “butyl” includes n-butyl, isobutyl, sec-butyl, and tert-butyl; and “propyl” includes n-propyl and isopropyl. In some embodiments, a deuterium atom maybe be a replacement for a hydrogen atom. The related term “alkylene” refers to a divalent radical derived from an alkyl group by removing at least two hydrogens from one or more carbons. The term “alkylene” includes straight chained and branched alkylene groups. The related term “cycloalkylene” refers to a divalent radical derived from a cycloalkyl group by removing hydrogen from two carbons.

When the alkyl or alkylene groups described herein are said to be “substituted,” they may be substituted with any substituent or substituents as those found in the exemplary compounds and embodiments disclosed herein, as well as deuterium, alkyl (including, e.g., cycloalkyl), keto (═O), aryl, heteroaryl, hydroxyl, alkoxy, alkyl sulfonamido, aryl sulfonamido, and halo. In certain embodiments, the optional substituents themselves may be optionally substituted with any of the substituents set forth in the exemplary compounds and embodiments set forth herein. For example, any of the alkyl groups set forth herein may be substituted with an aryl and/or heteroaryl group, wherein the aryl and heteroaryl groups themselves are optionally substituted with one or more groups selected from hydroxyl, halo, and C₁-C₈ alkoxy.

As used herein, the term “alkenyl” refers to an alkyl group that contains one or more carbon-carbon double bonds, including “cycloalkenyl” groups as defined below. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃), and —CH₂C≡C(CH₂CH₃), among others. When the alkenyl and alkynyl groups described herein are said to be “substituted,” they may be substituted with any substituent or substituents as those found in the exemplary compounds and embodiments disclosed herein, as well as those identified above as suitable substituents for substituted “alkyl” groups.

As used herein, the term “cycloalkenyl” refers to a cyclic alkyl group that is partially saturated. The related term “cycloalkanyl” refers to a cyclic alkyl group that is totally saturated.

As used herein, the term “alkoxy” refers to —O-(alkyl), wherein “alkyl” is as defined above and may be optionally substituted as defined above.

As used herein, the term “aryl” refers to an aromatic carbocyclic group of from 6 to 14 carbon atoms having a single ring (e.g., phenyl) or multiple condensed rings (e.g., naphthyl or anthryl). In some embodiments, aryl groups contain 6-14 carbons and in others from 6 to 12 or even 6 to 10 carbon atoms in the ring portions of the groups. Particular aryls include phenyl, biphenyl, naphthyl and the like. The phrase “aryl groups” also includes groups containing fused rings, such as fused aromatic-aliphatic ring systems (e.g., indanyl, tetrahydronaphthyl, and the like). When the aryl groups described herein are said to be “substituted,” they may be substituted with any substituent or substituents as those found in the exemplary compounds and embodiments disclosed herein, as well as deuterium, aryl, alkyl, heteroaryl, hydroxyl, and halo, as well as any of the substituents for substituted “alkyl” groups as noted above. The related term “arylene” references a bivalent residue derived from an aryl group by removing at removing a hydrogen atom from two ring carbon atoms.

As used herein, the term “heteroaryl” refers to an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. When the heteroaryl groups described herein are said to be “substituted,” they may be substituted with any substituent or substituents as those found in the exemplary compounds and embodiments disclosed herein, as well as deuterium, aryl, alkyl, heteroaryl, hydroxyl, and halo, as well as any of the substituents for substituted “alkyl” groups as noted above. The related term “heteroarylene” references a bivalent residue derived from a heteroaryl group by removing at removing a hydrogen atom from two ring carbon atoms.

As used herein, the term “heterocyclic ring” or “heterocyclyl” or “heterocycloalkyl” refers to a non-aromatic cycloalkyl in which one to four of the ring carbon atoms are independently replaced with a heteroatom selected from O, S, and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. When the heterocyclyl groups described herein are said to be “substituted,” they may be substituted with any substituent or substituents as those found in the exemplary compounds and embodiments disclosed herein, as well as deuterium, aryl, alkyl, heteroaryl, hydroxyl, and halo, as well as any of the substituents for substituted “alkyl” groups as noted above. The related term “heterocycloalkylene” references a divalent radical derived from a heterocyclic group by removing hydrogens from two carbons.

As used herein, the term “heteromoieties” refers to any groups containing a heteroatom, for example, amino, 0, Se, Se(O), SeO₂, S, S(O), and SO₂.

As used herein, the term “halo” or “halogen” refers to a fluorine, chlorine, bromine or iodine atom.

As used herein, the term “hydroxyl” refers to —OH group.

As used herein, the term “alkyl sulfonamido” refers to an alkyl moiety containing —S(═O)₂—NR₂, wherein each R group is chosen from an alkyl and H.

As used herein, the term “aryl sulfonamido” refers to an aryl moiety containing —S(═O)₂—NR₂, wherein each R group is chosen from an aryl and H.

In some embodiments, the compound of Formula I contains one or more stereocenters. In some circumstances, the compound of Formula I comprises a racemic mixture. In some embodiments, the compound of Formula I comprises the (S) enantiomer. In some embodiments, the compound of Formula I comprises the (R) enantiomer. In some embodiments, the (S) and (R) designations refer to the absolute stereochemistry of a compound having more than one stereocenter. In such cases, the conformation of one of those stereocenters may be referred to in terms of its relative (D) or (L) configuration. In some embodiments, the compounds of Formula I exhibit S,S absolute stereochemistry at the two stereocenters formed by Group A when the [symbol] is a single bond and Rₓ and R_{w} are present.

In some embodiments, X and Y are independently selected from hydrogen, deuterium, and optionally substituted C₁-C₈ alkyl, wherein the alkyl group comprises a cycloalkyl moiety (e.g., cyclopropyl, cyclobutyl, etc.). In some embodiments, at least one of X or Y is an unsubstituted C₁-C₄ alkyl. In some embodiments, at least one of X or Y is an unsubstituted C₂-C₄alkyl.

In some embodiments, R₂, R₃, R_{3′}, R₄, R₅, R_{w}, Rₓ, R₆ and R₇ are each independently selected from hydrogen, deuterium, halo, —N(R₉)₂, —SR₉, optionally substituted C₁-C₈ alkyl, —C₁—C alkoxy, and optionally substituted C₂-C₈ alkenyl, or Y is absent and R₃ taken together with carbon to which it is attached and the nitrogen atom to which X is attached form a 3- to 7-membered heterocyclic ring optionally including 1 to 2 additional ring heteromoieties selected from O, S, S(O), SO₂, and NR₉. In some embodiments, R₂, R₃, R_{3′}, R₄, R₅, R_{w}, Rₓ, R₆ and R₇ are each independently selected from hydrogen, deuterium, halo, optionally substituted C₁-C₈ alkyl, and optionally substituted C₂-C₈ alkenyl. In some embodiments, R₂ is selected from C₁-C₈ alkyl and C₂-C₈ alkenyl, each of which are optionally substituted with fluorine or deuterium. In some embodiments, R₂ is selected from C₂-C₈ alkyl and C₂-C₈ alkenyl, each of which are optionally substituted with fluorine or deuterium.

In some embodiments, R₃, R_{3′}, Rₓ and R_{w} are each, independently, selected from hydrogen, deuterium, —N(R₉)₂, —SR₉, halo, optionally substituted C₁-C₈ alkyl, —C₁-C₈ alkoxy, and optionally substituted C₂-C₈ alkenyl. In some embodiments, Rₓ and R_{w} are both absent. In some embodiments, at least one of R₃, R_{3′}, Rₓ or R_{w} is not hydrogen. In some embodiments, at least one of R₃, R_{3′}, Rₓ or R_{w} is deuterium. In some embodiments, at least one of R₃, R_{3′}, Rₓ or R_{w} is halo. In some embodiments, R₃ and R_{3′} are taken together with the carbon to which they are attached to form a keto group (═O). Exemplary compounds where R₃ and R_{3′} are taken together with the carbon to which they are attached to form a keto group include intermediates for certain target compounds disclosed herein, such as the target amide compound produced in accordance with Example 20 below.

In some embodiments, R₄ and R₅ are each independently selected from hydrogen, deuterium, —N(R₉)₂, —SR₉, optionally substituted C₁-C₈ alkyl, optionally substituted C₂-C₈ alkenyl, halo, hydroxyl, —C₁-C₈ alkoxy, —OC(O)R₈, —OC(O)OR, —OP(O)(OR₉)₂, and —OSO₂R₈. In some embodiments, R₄ and R₅ are each independently selected from hydrogen, deuterium, optionally substituted C₁-C₈ alkyl, optionally substituted C₂-C₈ alkenyl, halo, hydroxyl, —C₁-C₈ alkoxy, —OC(O)R₈, —OC(O)OR, —OP(O)(OR₉)₂, and —OSO₂R₈.

In certain embodiments, at least one of R₄ and R₅ is a C₁-C₈alkoxy group, or in some embodiments a C₂-C₄ alkoxy group, wherein it may be a straight chain or branched C₁-C₈alkoxy group or C₂-C₄ alkoxy group, for example a straight chain, and may be methoxy or ethoxy. In some embodiments, R₅ is C₁-C₈alkoxy. In some embodiments, R₄ is selected from hydrogen and fluorine, and R₅ is C₁-C₈alkoxy. In some embodiments, at least one of R₄ and R₅ is a C₁-C₈alkyl group or, in some embodiments, a C₁-C₄ alkyl group, for example a straight chain C₁-C₄ alkyl. In some embodiments, R₅ is selected from methyl, ethyl, n-propyl and n-butyl, and for example methyl or ethyl. In some embodiments, at least one of R₄ and R₅ is halo. In some embodiments, R₄ is fluoro. In some embodiments, R₄ is fluoro and R₅ is selected from hydrogen and C₁-C₈alkoxy. In some embodiments, at least one of R₄ and R₅ is —OC(O)R. In some embodiments R₄ is —OC(O)R₃ and R₅ is hydrogen or fluoro.

In some embodiments, R₃ is selected from optionally substituted C₁-C₈ alkyl, optionally substituted C₂-C₈ alkenyl, and optionally substituted aryl. In some embodiments, R₉ is selected from hydrogen, deuterium, optionally substituted C₁-C₈ alkyl, optionally substituted C₂-C₈ alkenyl, and optionally substituted aryl. In some embodiments, R₃ is selected from methyl, ethyl, propyl, and isopropyl. In some embodiments, R₉ is selected from methyl, ethyl, propyl, and isopropyl.

Exemplary halo residues for compounds of Formula I include chloro, bromo, fluoro, and iodo. In certain embodiments, the compounds of Formula I comprise at least one fluoro residue.

In some embodiments, W₁ is selected from CF₂, NR₁, O, S, S(O), SO₂, Se, Se(O), and SeO₂. In some embodiments, W₁ is NR₁. In some embodiments, W₁ is 0. In some embodiments, W₁ is S. In some embodiments, W₁ is Se.

In some embodiments, Z₆ is selected from N and CR₆; and Z₇ is selected from N and CR₇. In some embodiments, Z₆ is N. In some embodiments, Z₇ is N.

In some embodiments, R₁ is selected from hydrogen, deuterium, optionally substituted C₁-C₈ alkyl, optionally substituted C₂-C₈ alkenyl, —C(O)R₃, —C(O)OR₃, —P(O)(OR₉)₂, —C(O)N(R₉)₂, —SOR₃, and —SO₂R₈. In some embodiments, R₁ is hydrogen. In some embodiments, R₁ is optionally substituted C₁-C₈ alkyl. In some embodiments, R₁ is optionally substituted C₂-C₄alkyl. In some embodiments, R₁ is methyl. In some embodiments, R₁ is ethyl. In some embodiments, R₁ is isopropyl.

In certain embodiments, the alkyl groups of Formula I are selected from C₁-C₈ alkyl, C₂-C₈alkyl, C₃-C₈ alkyl, and C₄-C₈ alkyl, or methyl, ethyl, propyl, isopropyl, n-butyl, isobutyl, sec-butyl, tert-butyl pentyl, isopentyl, hexyl, heptyl, octyl, etc. In certain embodiments, the alkenyl groups of Formula I are selected from C₂-C₈ alkenyl, C₃-C₈ alkenyl, and C₄-C₈ alkenyl, or ethenyl, propenyl, butenyl, pentenyl, hexenyl, heptenyl, octenyl, etc.

In certain embodiments, the alkyl, alkenyl or alkynyl groups of Formula I may be unsubstituted or substituted with one or more groups selected from deuterium, aryl, heteroaryl, hydroxyl, alkoxy, alkyl sulfonamido, aryl sulfonamido, and halo.

In certain embodiments, the cycloalkenyl and heterocyclyl groups of Formula I may be unsubstituted or substituted with one or more groups selected from deuterium, alkyl, alkenyl, aryl, heteroaryl, hydroxyl, alkoxy, alkyl sulfonamido, aryl sulfonamido, and halo.

In certain embodiments, the aryl and heteroaryl groups of Formula I may be unsubstituted or substituted with one or more groups selected from deuterium, aryl, alkyl, heteroaryl, hydroxyl, and halo. In certain embodiments, the alkoxy groups of Formula I may be unsubstituted or substituted with one or more groups selected from aryl, alkyl, heteroaryl, hydroxyl, and halo.

In some embodiments, X and/or Y are a straight chain C₁-C₄ alkyl or a C₂-C₄ alkenyl. In some embodiments, X and Y are each methyl, X and Y are each ethyl, or X is methyl and Y is ethyl. In certain embodiments, X and/or Y are an C₁-C₈ alkyl or C₂-C₈ alkenyl optionally substituted with at least one halo group, such as fluorine. In certain embodiments, at least one of X or Y is a group selected from —CF₃, —CHF₂, —CH₂F, —CH₂CF₃, —CH₂CHF₂, and —CH₂CH₂F. In certain embodiments, at least one of X or Y is a group selected from -CD₃, —CH₂CD₃, -CD₂CH₃, and -CD2CD3.

In some embodiments, X is unsubstituted C₁-C₈ alkyl. In some embodiments, X is methyl. In some embodiments, X is ethyl. In some embodiments, X is n-propyl. In some embodiments, X is isopropyl. In some embodiments, X is cyclopropyl. In some embodiments, Y is hydrogen. In some embodiments, Y is unsubstituted C₁-C₈ alkyl. In some embodiments, Y is methyl. In some embodiments, Y is ethyl. In some embodiments, Y is n-propyl. In some embodiments, Y is isopropyl. In some embodiments, Y is cyclopropyl.

In some embodiments, R₂, R₃, R_{3′}, R_{w}, Rₓ, R₆ and R₇ are each independently selected from hydrogen, deuterium, halo, and C₁-C₄ alkyl, for example a straight chain C₁-C₄ alkyl. In some embodiments, R₂, R₃, R_{3′}, R_{w}, Rₓ, R₆ and R₇ are each independently selected from hydrogen, deuterium, halo, methyl, ethyl, n-propyl, isopropyl, n-butyl, and isobutyl. In other embodiments, R₂, R₃, R_{3′}, R_{w}, Rₓ, R₆ and R₇ are each independently selected from hydrogen, deuterium, methyl, and ethyl. In some embodiments, R₂ is selected from optionally substituted methyl, ethyl, propyl, and butyl. In some embodiments, R₂ is optionally substituted propyl, such as n-propyl optionally substituted with one or more deuterium atoms.

In some embodiments, R₂ is a C₁-C₄ alkyl group substituted with at least one group selected from deuterium, halo, aryl, and heteroaryl, wherein the aryl and heteroaryl groups are themselves optionally substituted. In some embodiments, R₂ is a C₂-C₄ alkenyl group substituted with at least one group selected from deuterium, halo, aryl, and heteroaryl, wherein the aryl and heteroaryl groups are themselves optionally substituted. In some embodiments, R₂ is a C₁-C₄ alkyl group substituted with at least one deuterium and at least one fluoro group. In some embodiments, R₂ is a C₂-C₄ alkenyl group substituted with at least one deuterium and at least one fluoro group.

In some embodiments, R₃ and R_{3′} are hydrogen. In some embodiments, R₃ and R_{3′} are each independently selected from hydrogen, methyl, and ethyl. In some embodiments, R₃ is hydrogen. In some embodiments, R₃ is methyl and R₃ is hydrogen. In some embodiments, R₃ and R₃, are both hydrogen. In some embodiments, R₃ and R_{3′} are both deuterium. In some embodiments, R₃ is hydrogen and R₃ is deuterium. In some embodiments, when R₃ and R_{3′} are not the same, it represents a stereocenter wherein the compound of Formula I comprises a racemic mixture. In some embodiments, when R₃ and R_{3′} are not the same or when R₃ is not hydrogen, it represents a stereocenter wherein the compound of Formula I comprises the (S) enantiomer. In some embodiments, when R₃ and R_{3′} are not the same or when R₃, is not hydrogen, it represents a stereocenter wherein the compound of Formula I comprises the (R) enantiomer. In some embodiments, a racemic mixture can be resolved to provide a pure enantiomer or a mixture enhanced with either the (R) or (S) enantiomer.

In some embodiments, R₆ and R₇ are each independently selected hydrogen, halo, methyl, ethyl, n-propyl, isopropyl, n-butyl, and isobutyl. In some embodiments, R₆ is selected from hydrogen and halo. In some embodiments, R₆ is selected from hydrogen and fluorine. In some embodiments, R₆ is fluorine. In some embodiments, R₆ is selected from alkoxy and halo.

In some embodiments, R₆ is selected from methoxy, chloro, and fluoro. In some embodiments, R₆ is fluorine. In some embodiments, R₆ is methoxy. In some embodiments, R₇ is selected from hydrogen and optionally substituted C₁-C₄ alkyl. In some embodiments, R₇ is selected from hydrogen, methyl, and ethyl. In some embodiments, R₇ is optionally substituted C₁-C₄ alkyl. In some embodiments, when R₆ is fluoro, then R₇ is selected from hydrogen and optionally substituted C₁-C₄ alkyl.

In certain embodiments, R₅ is selected from hydrogen, C₁-C₈ alkoxy, and halo. In certain embodiments, R₅ is hydrogen. In certain embodiments, R₅ is methoxy. In certain embodiments, R₄ is selected from hydrogen, C₁-C₈ alkoxy, hydroxyl, and —OC(O)R₃. In certain embodiments, R₄ is hydrogen. In certain embodiments, R₄ is hydroxyl. In certain embodiments, R₄ is —OC(O)R₃.

In certain embodiments, R₁ is hydrogen. In certain embodiments, R₁ is C₁-C₄ alkyl. In certain embodiments, R₁ is methyl.

In certain embodiments, R₂ is hydrogen.

In certain embodiments, X and Y are selected from hydrogen and deuterium. In certain embodiments, X is hydrogen and Y is C₁-C₈ alkyl. In certain embodiments, X is hydrogen. In certain embodiments, Y is selected from methyl, ethyl, and n-propyl. In certain embodiments, X is methyl.

In certain embodiments, one or more hydrogen atoms on compounds of Formula I may be replaced with one or more deuterium atoms. For example, in certain embodiments, R₆ may comprise a deuterium atom as a replacement for a hydrogen, or when R₇ is a —CH₃, each hydrogen atom may be replaced to form a -CD₃ residue. Similarly, another non-limiting example includes when X and/or Y is a —CH₃, each hydrogen atom may be replaced to form a -CD₃ residue.

In some embodiments,

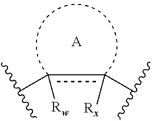

(also referred to as “Group A”) represents a fused ring (in other words, a ring system fused to the rest of the molecular scaffold), and may be chosen from an optionally substituted cycloalkanyl, an optionally substituted cycloalkenyl, optionally substituted heterocyclyl that is partially saturated, optionally substituted aryl, and optionally substituted heteroaryl. In certain embodiments, the Group A ring system itself may comprise a fused ring system (e.g., optionally substituted aryl such as naphthalene or indole). In certain embodiments, Group A is selected from the following:

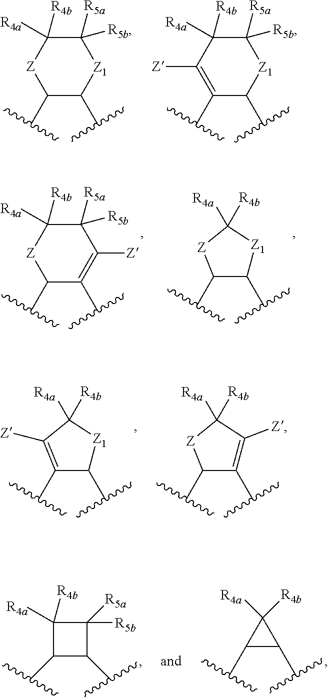

wherein Z and Z₁ are independently selected from O, S and —C(Z′)₂—, and R₄ₐ, R_{4b}, R₅ₐ, R_{5b} and Z′ are each independently selected from hydrogen, deuterium, halogen, hydroxyl, branched or unbranched C₁-C₈ alkyl, and branched or unbranched C₂-C₈ alkenyl.

In certain embodiments, Group A may be unsubstituted or optionally substituted with one or more groups selected from deuterium, alkyl, alkenyl, aryl, heteroaryl, hydroxyl, alkoxy, alkyl sulfonamido, aryl sulfonamido, and halo. In some embodiments, Group A may be substituted with one or more groups selected from C₁-C₈ alkyl or C₂-C₈ alkenyl, such as methyl, ethyl, n-propyl, isopropyl, and allyl.

In some embodiments, the compounds of Formula I are represented by the subgenus Formula II:

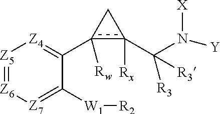

wherein all variables are as defined above.

In certain embodiments for compounds of Formulae I and II, Z₅ is CR₅, and R₅ is selected from hydrogen, C₁-C₈ alkoxy, and halo. In certain embodiments, Z₅ is CR₅, and R₅ is hydrogen. In certain embodiments, Z₅ is CR₅, and R₅ is methoxy. In certain embodiments, Z₅ is CR₅, and R₅ is halo, such as fluoro.

In certain embodiments, Z₄ is CR₄, Z₅ is CR₅, Z₆ is CR₆ and Z₇ is CR₇, wherein R₅ is halo, R₄ is selected from hydrogen, deuterium and halo, R₆ is selected from hydrogen, deuterium and halo, and R₇ is selected from hydrogen, deuterium and halo.

In certain embodiments, Z₄ is CR₄, Z₅ is CR₅, Z₆ is CR₆ and Z₇ is CR₇, wherein R₅ is fluoro, R₄ is selected from hydrogen, deuterium and halo, R₆ is selected from hydrogen, deuterium and halo, and R₇ is selected from hydrogen, deuterium and halo.

In certain embodiments, Z₄ is CR₄, Z₅ is CR₅, Z₆ is CR₆ and Z₇ is CR₇, wherein R₅ is fluoro, R₄ is selected from hydrogen, deuterium and fluoro, R₆ is selected from hydrogen, deuterium and fluoro, and R₇ is selected from hydrogen, deuterium and fluoro.

In certain embodiments, Z₄ is CR₄, Z₅ is CR₅, Z₆ is CR₆ and Z₇ is CR₇, wherein R₅ is fluoro, R₄ is selected from hydrogen, deuterium and fluoro, R₆ is selected from hydrogen, deuterium and fluoro, and R₇ is selected from hydrogen and deuterium.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen; W₂ is C(R₃)(R_{3′}) and R₃ and R_{3′} are each selected from hydrogen and deuterium; and X and Y are each hydrogen.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen; W₂ is C(R₃)(R_{3′}) and R₃ and R_{3′} are each hydrogen; X and Y are each hydrogen; [symbol] is a single bond, wherein Rₓ and R_{w} are each independently selected from hydrogen, deuterium, and fluoro; and W₁ is selected from S and O.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen; W₂ is C(R₃)(R_{3′}) and R₃ and R_{3′} are each hydrogen; X and Y are each hydrogen; [symbol] is a single bond, wherein Rₓ and R_{w} are each hydrogen; W₁ is selected from S and O; and R₂ is selected from a group selected from C₁-C₈ alkyl, C₂-C₈ alkenyl, aryl, and heteroaryl, wherein said group is substituted with at least one of deuterium or fluoro.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen; W₂ is C(R₃)(R_{3′}) and R₃ and R_{3′} are each hydrogen; X and Y are each hydrogen; [symbol] is a single bond, wherein Rₓ and R_{w} are [symbol] each hydrogen; W₁ is selected from S and O; and R₂ is selected from a group selected from C₁-C₈ alkyl, C₂-C₈ alkenyl, aryl, and heteroaryl, wherein said group is substituted with at least two deuterium atoms.

In certain embodiments, Z₄ is CR₄, Z₅ is CR₅, Z₆ is CR₆ and Z₇ is CR₇, wherein R₅ is fluoro, R₄ is hydrogen, R₆ is hydrogen, and R₇ is hydrogen; W₂ is C(R₃)(R_{3′}) and R₃ and R_{3′} are each hydrogen; X and Y are each hydrogen; [symbol] is a single bond, wherein Rₓ and R_{w} are each hydrogen; W₁ is selected from S and O; and R₂ is selected from a group selected from C₁-C₄ alkyl and C₂-C₄ alkenyl, wherein said group is substituted with at least two deuterium atoms.

In certain embodiments, R₂ is a residue selected from Formula III:

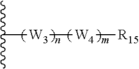

wherein
   W₃ is, for each occurrence, independently selected from arylene, heteroarylene, cycloalkylene, heterocycloalkylene, C(R₁₀)(R₁₁), and

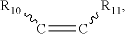

wherein n is an integer selected from 1 to 10;
W₄ is, for each occurrence, independently selected from O, S, Se, arylene, heteroarylene, cycloalkylene, heterocycloalkylene, C(R₁₂)(R₁₃), and

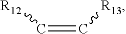

wherein m is an integer selected from 0 to 10; and
R₁₀, R₁₁, R₁₂, R₁₃, and R₁₅ are, for each occurrence, independently selected from hydrogen, deuterium, halo, and hydroxyl.

In certain embodiments of Formula I wherein W₂ is C(R₃)(R_{3′}) and R₂ is a residue of Formula III where W₃ is C(R₁₀)(R₁₁), at least one of R₃, R_{3′}, R₁₀ or R₁₁ is deuterium.

In certain embodiments of Formula I wherein W₂ is C(R₃)(R_{3′}) and R₂ is a residue of Formula III where W₃ is C(R₁₀)(R₁₁) and n is 1, at least one of R₃, R_{3′}, R₁₀ or R₁₁ is deuterium.

In certain embodiments, W₃ is, for each occurrence, independently selected from C(R₁₀)(R₁₁), wherein n is an integer selected from 1 to 4, and at least one R₁₀ is deuterium.

In certain embodiments, W₃ is, for each occurrence, independently selected from C(R₁₀)(R₁₁), wherein n is an integer selected from 1 to 4, and at least one R₁₀ and at least one R₁ are deuterium.

In certain embodiments, W₃ is C(R₁₀)(R₁₁), wherein n is 1, and R₁₀ and R₁₁ are both deuterium.

In certain embodiments, W₃ is C(R₁₀)(R₁₁), wherein n is 1, and R₁₀ and R₁₁ are both deuterium; W₄ is, for each occurrence, independently selected from arylene, heteroarylene, C(R₁₂)(R₁₃), and

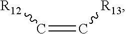

wherein m is an integer selected from 0 to 3, R₁₂ and R₁₃ are each independently selected from hydrogen, deuterium, and halo for each occurrence; and R₁₅ is selected from hydrogen, halo, and deuterium.

In certain embodiments, W₃ is C(R₁₀)(R₁₁), wherein n is 1, and R₁₀ and R₁₁ are both deuterium; W₄ is, for each occurrence, independently selected from C(R₁₂)(R₁₃) and

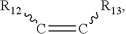

wherein m is an integer selected from 0 to 3, R₁₂ and R₁₃ are each independently selected from hydrogen, deuterium, and fluoro for each occurrence; and R₁₅ is selected from hydrogen, fluoro, and deuterium.

In certain embodiments, W₃ is C(R₁₀)(R₁₁), wherein n is 1, and R₁₀ and R₁₁ are both deuterium; W₄ is, for each occurrence, independently selected from C(R₁₂)(R₁₃) and

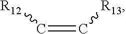

wherein m is an integer selected from 0 to 3, R₁₂ and R₁₃ are each independently selected from hydrogen and fluoro for each occurrence; and R₁₅ is selected from hydrogen and fluoro.

In certain embodiments, at least one occurrence of R₁₀, R₁₁, R₁₂, R₁₃ or R₁₅ is deuterium.

For example, in certain embodiments when n is 1, one or both of R₁₀ and R₁₁ are deuterium. In certain embodiments when n is 1 and one or both of R₁₀ and R₁₁ are deuterium, m is an integer selected from 1 to 10 and each occurrence of R₁₂ and R₁₃ are selected from hydrogen and halo.

In certain embodiments, R₁₅ is selected from hydrogen, deuterium, and halo. In certain embodiments, R₁₅ is fluoro. In certain embodiments, at least one of R₁₂ or R₁₃ is fluoro.

In certain embodiments, at least one of R₁₀, R₁₁, R₁₂, R₁₃, R₃ or R_{3′} is deuterium. In certain embodiments, at least two groups selected from R₁₀, R₁₁, R₁₂, R₁₃, R₃ and R_{3′} are deuterium. In certain embodiments, at least four groups selected from R₁₀, R₁₁, R₁₂, R₁₃, R₃ and R_{3′} are deuterium.

In certain embodiments, at least one of R₁₀, R₁₁, R₃ or R_{3′} is deuterium. In certain embodiments, at least two groups selected from R₁₀, R₁₁, R₃ and R_{3′} are deuterium. In certain embodiments, R₁₀, R₁₁, R₃ and R_{3′} are all deuterium.

In certain embodiments, R₃ and R_{3′} are hydrogen. In certain embodiments, R₃ and R₃ are deuterium. In some embodiments, R₂ is an unsubstituted C₁-C₄ alkyl, such as n-propyl. In some embodiments, R₂ is a substituted C₁-C₄ alkyl, wherein the substituents are selected from deuterium and fluoro. In some embodiments, R₂ is a C₁-C₄ alkyl substituted with at least two deuteriums. In some embodiments, R₂ is a C₁-C₄ alkyl substituted with at least four deuteriums.

In some embodiments, R₃ and R_{3′} are hydrogen and R₂ is a C₁-C₄ alkyl substituted with at least two deuteriums. In some embodiments, R₃ and R_{3′} are deuterium and R₂ is an unsubstituted C₁-C₄ alkyl (e.g., n-propyl). In some embodiments, R₃ and R₃ are deuterium and R₂ is a C₁-C₄ alkyl substituted with at least two deuteriums.

Exemplary compounds of Formula I include:

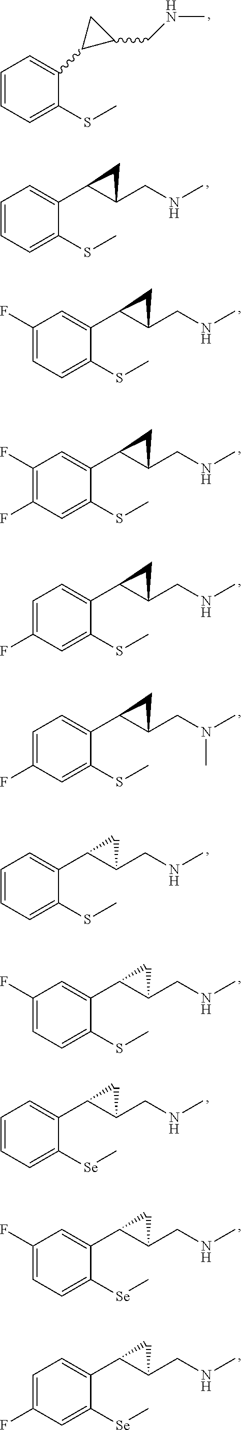

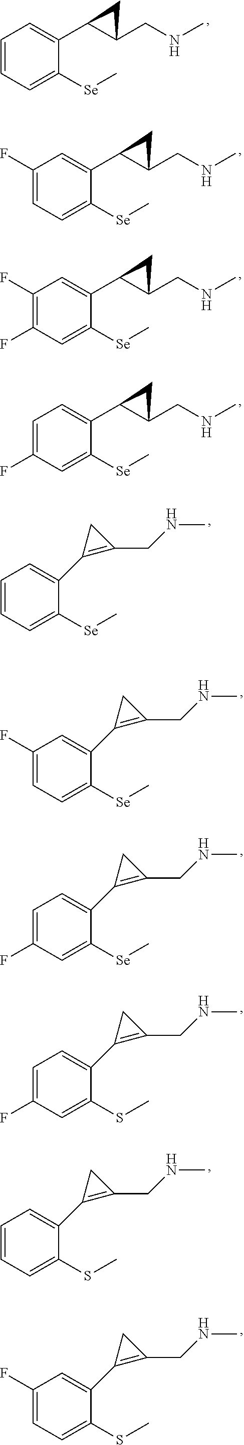

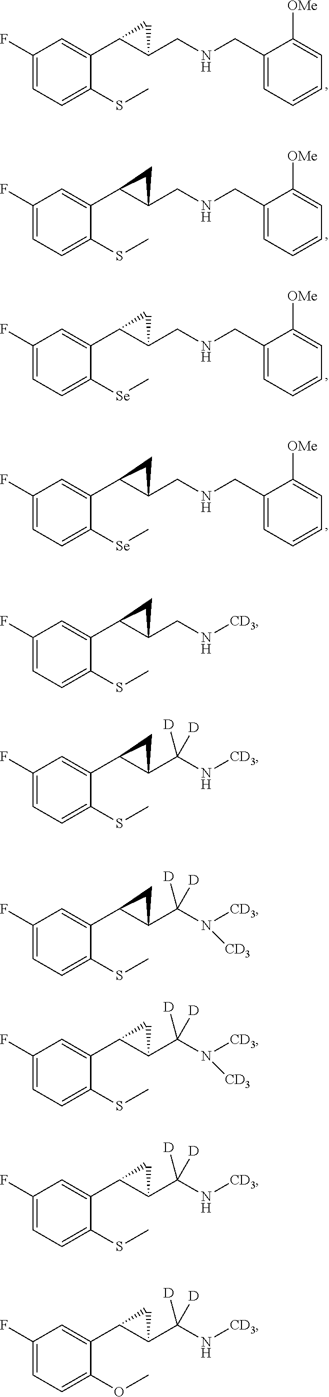

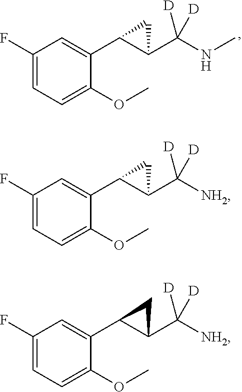 [SMILES: [2H]C([2H])(NC)[C@@H]1C[C@H]1c1cc(F)ccc1OC ; [2H]C([2H])(N)[C@@H]1C[C@H]1c1cc(F)ccc1OC ; [2H]C([2H])(N)[C@H]1C[C@@H]1c1cc(F)ccc1OC]

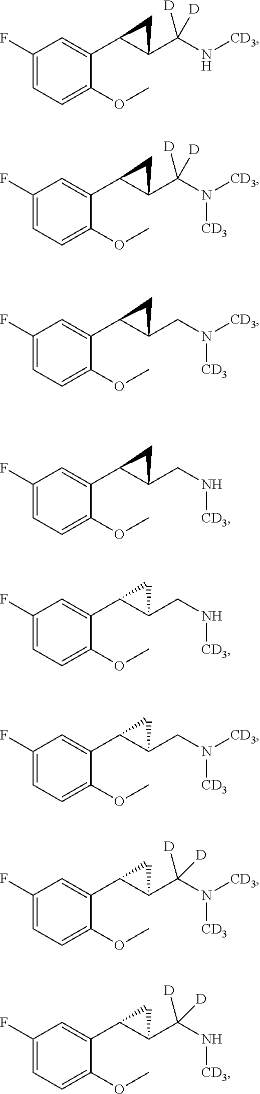

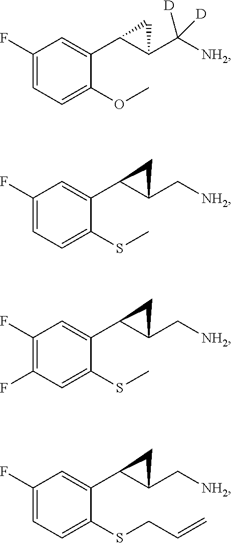 [SMILES: [2H]C([2H])(N)[C@@H]1C[C@H]1c1cc(F)ccc1OC ; CSc1ccc(F)cc1[C@H]1C[C@@H]1CN ; CSc1cc(F)c(F)cc1[C@H]1C[C@@H]1CN ; C=CCSc1ccc(F)cc1[C@H]1C[C@@H]1CN]

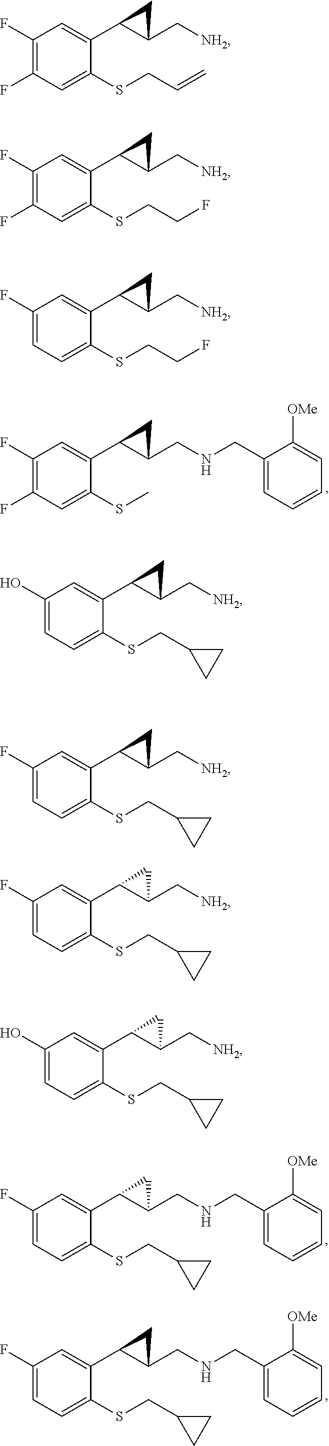

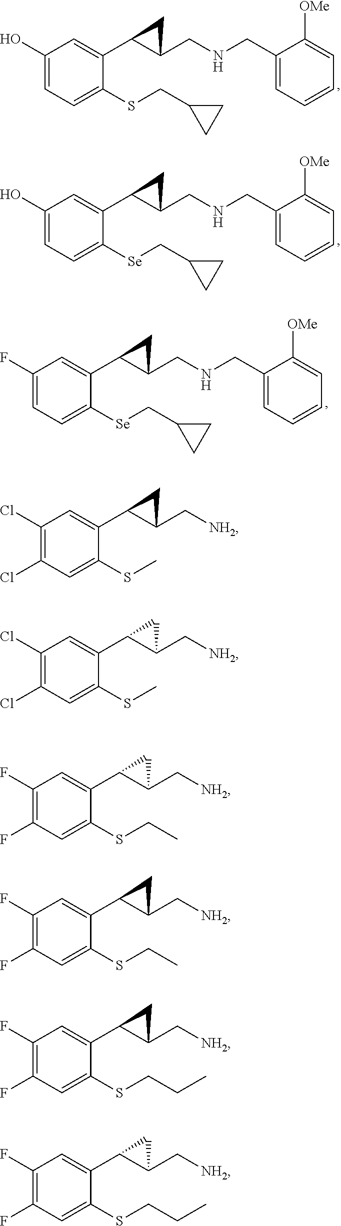

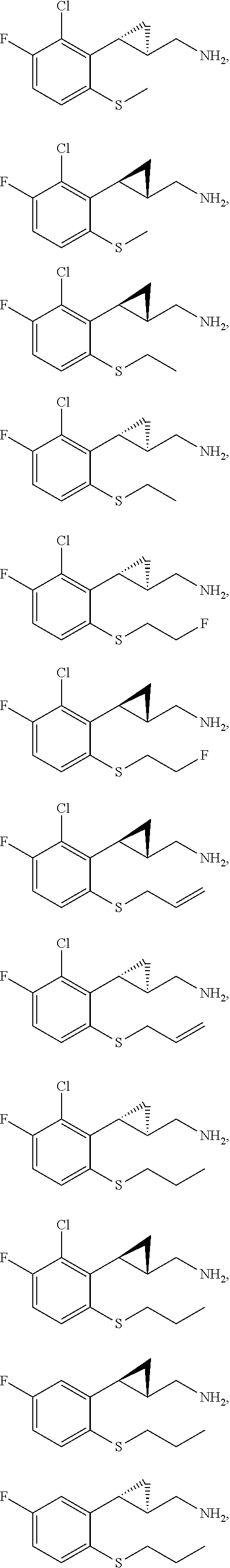

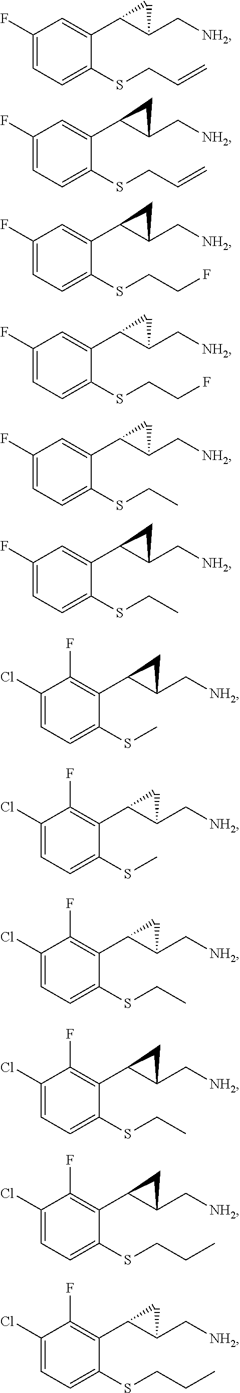

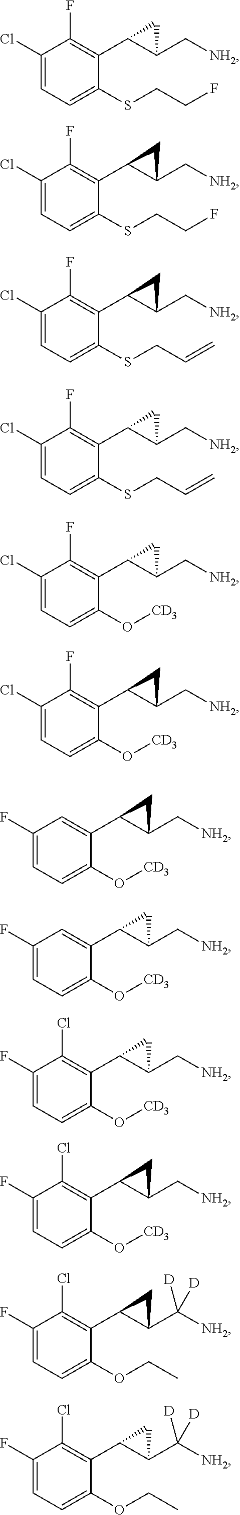

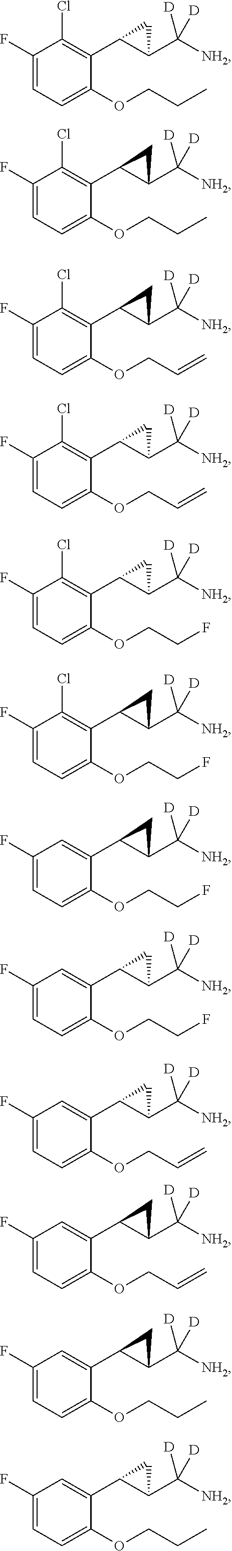

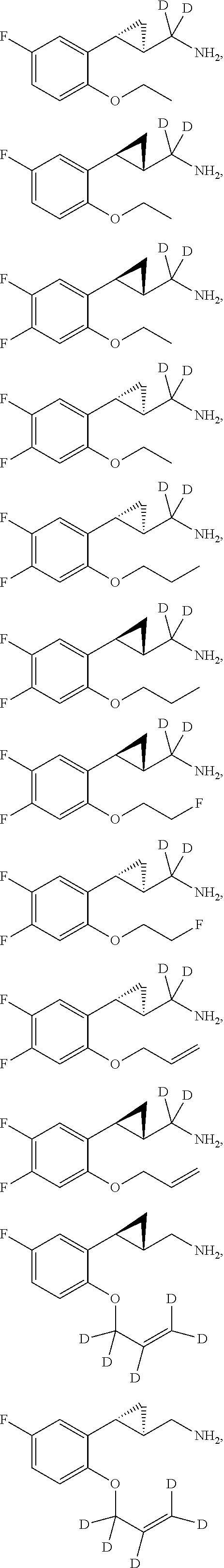

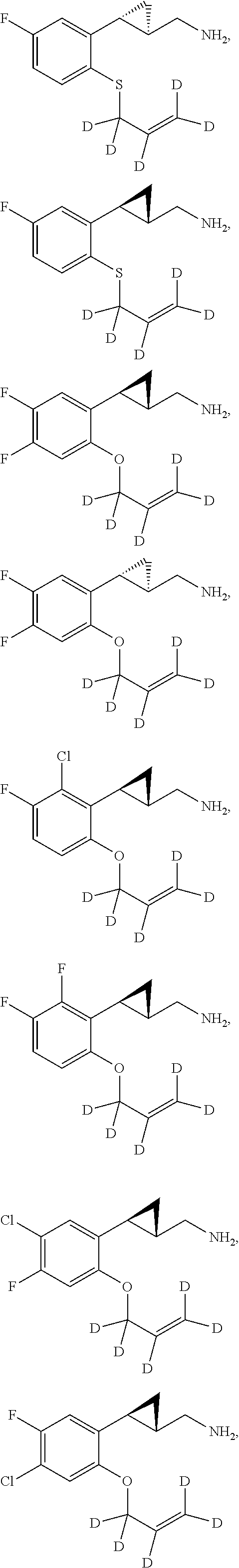

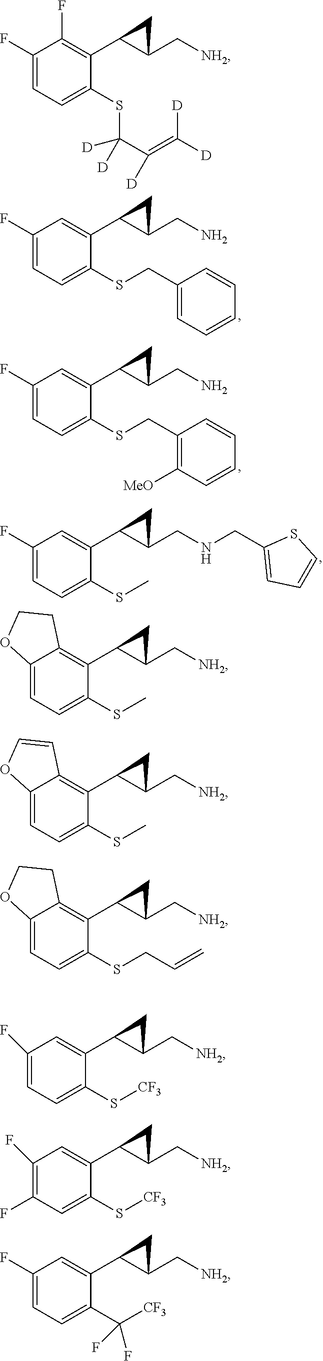

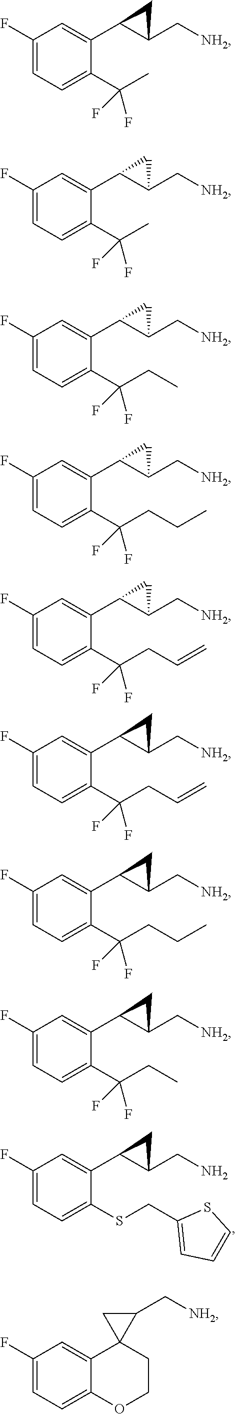

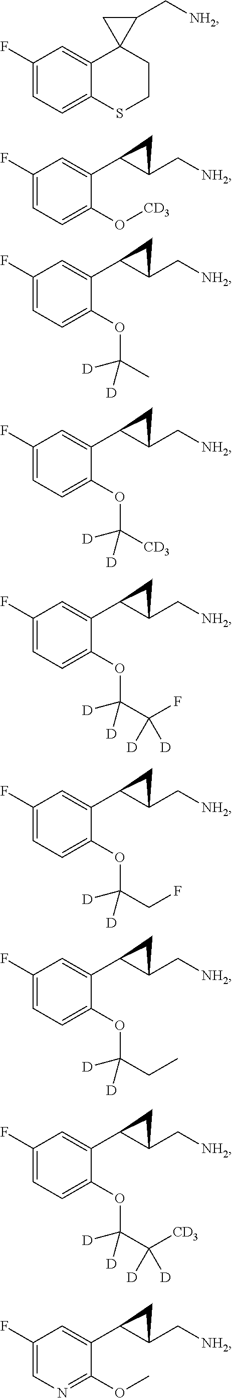

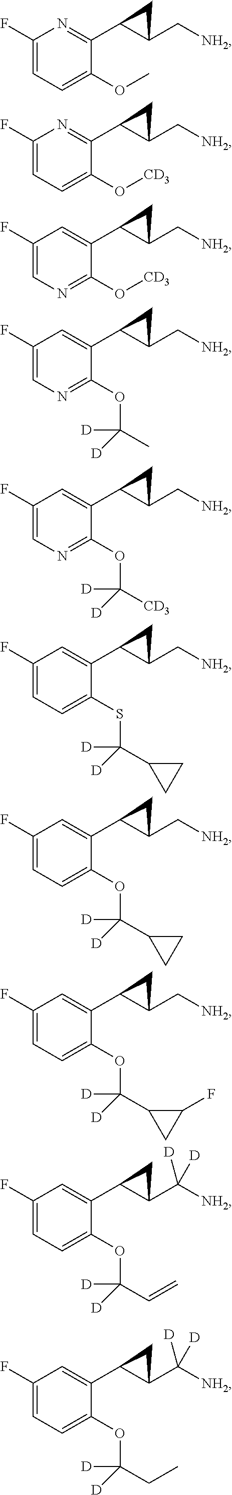

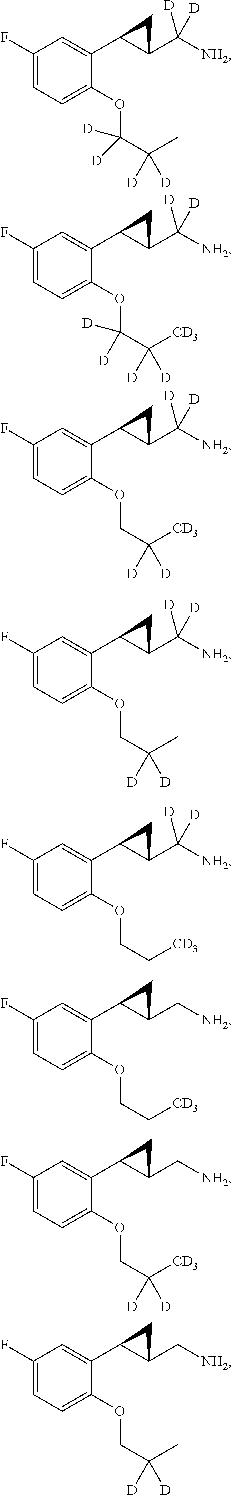

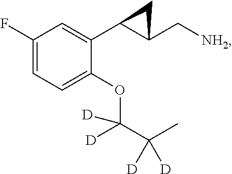 [SMILES: [2H]C([2H])(C)C([2H])([2H])Oc1ccc(F)cc1[C@H]1C[C@@H]1CN]

and salts, solvates, hydrates, prodrugs, and enantiomers of any of the foregoing compounds.

In some embodiments, the compound is Lumocaserin, which has the following structure:

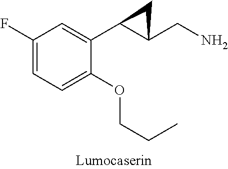 [SMILES: CCCOc1ccc(F)cc1[C@H]1C[C@@H]1CN]

and salts, solvates, hydrates, prodrugs, and enantiomers thereof. In some embodiments, the compound of Formula I is a deuterated Lumocaserin.

In some embodiments, the compounds of Formula I comprise salts. In some embodiments, the compounds of Formula I comprise pharmaceutically-acceptable salts. Exemplary salts include, but are not limited to, HCl, HI, HBr, HF, ascorbate, hydrofumarate, fumarate, oxalate, maleate, and the like. In certain embodiments, the compound of Formula I is in its free-base form. In some embodiments, the compound of Formula I comprises a salt, such as a [1:1] salt (e.g., HCl, hydrofumarate) or a [2:1] salt (e.g., oxalate, fumarate). For the [1:1] salts, one ammonium cation of one compound of Formula I is balanced by a single anion (Cl—, I—, etc.). For the [2:1] salts, two ammonium cations of two molecules of Formula I are balanced by a dianionic species, such as a dianion derived from di-acids such as oxalic acid and fumaric acid. Other exemplary salts include zwitterionic forms of compounds of Formula I, such as when R₁ is —P(O)(OH)₂, wherein deprotonation of an —OH on R₁ may result in intramolecular coordination of the resulting —O⁻ with the quaternary ethylammonium residue (e.g., —(CH₂)₂N⁺H(CH₃)₂).

In certain embodiments, the compounds described herein are hydrates. In some embodiments, the compounds described herein are non-stoichiometric hydrates. In some embodiments, the compounds described herein are ion-coordinated hydrates. In some embodiments, the compounds described herein are channel hydrates. In some embodiments, the compounds described herein pore hydrates. In certain embodiments, ion-coordinated hydrates, pore hydrates, and/or channel hydrates may be either stoichiometric or non-stoichiometric hydrates. In some embodiments, the hydrates are hydrated salt forms of compounds described herein (e.g., non-stoichiometric hydrates of Formula I HCl salts).

As used herein, the term “non-stoichiometric hydrate” denotes a crystalline solid in which water molecules occupy disordered or partially occupied sites within the crystal lattice or reside in lattice channels or voids, such that the overall water content is variable rather than constant. A non-stoichiometric hydrate retains its crystallinity and lattice structure across a range of water contents, and is therefore distinguishable from a stoichiometric hydrate, in which water occupies well-defined and fixed stoichiometric positions. For purposes of the present disclosure, the term “non-stoichiometric hydrate” includes partial hydrates, i.e., hydrates containing less than one mole of water per mole of compound, including but not limited to hydrates having 0.01 to 0.99 moles of water per mole of compound.

As used herein, the term “channel hydrate” refers to a crystalline hydrate in which one or more water molecules reside within continuous one-dimensional or two-dimensional channels, tunnels, or grooves formed by the packing arrangement of the compound in the crystal lattice. The water molecules may be ordered or disordered and may interact with the lattice through hydrogen bonding, van der Waals forces, electrostatic interactions, or ion coordination. Channel hydrates may be stoichiometric or non-stoichiometric and may exhibit reversible water uptake or release as a function of temperature or relative humidity while retaining the same fundamental crystalline framework. Channel hydrates may be identified by visualization of the extended crystalline network. Additionally, or in the alternative, channel hydrates may be identified by one or more analytical characteristics, including gradual and reversible water uptake in dynamic vapor sorption (DVS) analysis, variable but reproducible water content by Karl Fischer titration or thermogravimetric analysis, and substantially consistent XRPD patterns despite changes in hydration level.

As used herein, the term “pore hydrate” (also known as a “void” or “cage” hydrate) refers to a crystalline hydrate in which water molecules occupy isolated voids, cavities, or pockets within the crystal lattice that do not form continuous channels. The enclosed water molecules are typically stabilized by local hydrogen bonding or electrostatic interactions but are spatially confined and not part of a continuous network through the lattice. In some embodiments, pore/cage hydrates may more commonly exhibit fixed (stoichiometric) water content and may undergo stepwise dehydration accompanied by phase transitions or loss of crystallinity when water is removed.

As used herein, the term “ion-coordinated hydrate” refers to a crystalline solid form in which one or more water molecules are coordinated to ionic species present in the lattice, including cations (e.g., protonated amines, alkali metals, alkaline earth metals) or anions (e.g., halides, sulfonates, phosphates). Coordination may occur through hydrogen bonding, electrostatic interaction, or direct coordination and may involve water molecules located at defined lattice sites or within lattice channels, pores, or voids. An ion-coordinated hydrate may be stoichiometric or non-stoichiometric and includes hydrates in which water contributes to the stabilization of ionic interactions within the crystal lattice.

Other exemplary compounds include compounds of Formula II(a) set forth below in Table 1:

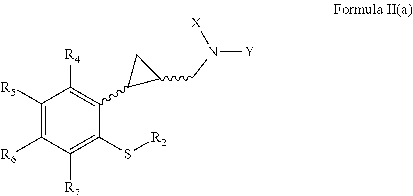

TABLE 1

| Ref. | X | Y | R₂ | R₄ | R₅ | R₆ | R₇ |
|---|---|---|---|---|---|---|---|
| 1 | —CH₃ | —CH₃ | —CH₃ | —OH | H | H | H |
| 2 | —CH₃ | —CH₂CH₃ | —CH₃ | —OH | H | H | H |
| 3 | —CH₃ | —(CH₂)₂CH₃ | —CH₃ | —OH | H | H | H |
| 4 | —CH₃ | —CH(CH₃)₂ | —CH₃ | —OH | H | H | H |
| 5 | —CH₃ | —(CH₂)₃CH₃ | —CH₃ | —OH | H | H | H |
| 6 | —CH₃ | —CH₂—HC═CH₂ | —CH₃ | —OH | H | H | H |
| 7 | —CH₂CH₃ | —CH₂CH₃ | —CH₃ | —OH | H | H | H |
| 8 | —CH₂CH₃ | —(CH₂)₂CH₃ | —CH₃ | —OH | H | H | H |
| 9 | —CH₂CH₃ | —CH(CH₃)₂ | —CH₃ | —OH | H | H | H |
| 10 | —CH₂CH₃ | —(CH₂)₃CH₃ | —CH₃ | —OH | H | H | H |
| 11 | —CH₂CH₃ | —CH₂—HC═CH₂ | —CH₃ | —OH | H | H | H |
| 12 | —(CH₂)₂CH₃ | —(CH₂)₂CH₃ | —CH₃ | —OH | H | H | H |
| 13 | —(CH₂)₂CH₃ | —CH(CH₃)₂ | —CH₃ | —OH | H | H | H |
| 14 | —(CH₂)₂CH₃ | —(CH₂)₃CH₃ | —CH₃ | —OH | H | H | H |
| 15 | —(CH₂)₂CH₃ | —CH₂—HC═CH₂ | —CH₃ | —OH | H | H | H |
| 16 | —CH(CH₃)₂ | —CH(CH₃)₂ | —CH₃ | —OH | H | H | H |
| 17 | —CH(CH₃)₂ | —(CH₂)₃CH₃ | —CH₃ | —OH | H | H | H |
| 18 | —CH(CH₃)₂ | —CH₂—HC═CH₂ | —CH₃ | —OH | H | H | H |
| 19 | —(CH₂)₃CH₃ | —(CH₂)₃CH₃ | —CH₃ | —OH | H | H | H |
| 20 | —(CH₂)₃CH₃ | —CH₂—HC═CH₂ | —CH₃ | —OH | H | H | H |
| 21 | —CH₂—HC═CH₂ | —CH₂—HC═CH₂ | —CH₃ | —OH | H | H | H |
| 22 | —CH₃ | —CH₂CH₃ | —CH₃ | —OC(O)CH₃ | H | H | H |
| 23 | —CH₃ | —(CH₂)₂CH₃ | —CH₃ | —OC(O)CH₃ | H | H | H |
| 24 | —CH₃ | —CH(CH₃)₂ | —CH₃ | —OC(O)CH₃ | H | H | H |
| 25 | —CH₃ | —(CH₂)₃CH₃ | —CH₃ | —OC(O)CH₃ | H | H | H |
| 26 | —CH₃ | —CH₂—HC═CH₂ | —CH₃ | —OC(O)CH₃ | H | H | H |
| 27 | —CH₂CH₃ | —CH₂CH₃ | —CH₃ | —OC(O)CH₃ | H | H | H |
| 28 | —CH₂CH₃ | —(CH₂)₂CH₃ | —CH₃ | —OC(O)CH₃ | H | H | H |
| 29 | —CH₂CH₃ | —CH(CH₃)₂ | —CH₃ | —OC(O)CH₃ | H | H | H |
| 30 | —CH₂CH₃ | —(CH₂)₃CH₃ | —CH₃ | —OC(O)CH₃ | H | H | H |
| 31 | —CH₂CH₃ | —CH₂—HC═CH₂ | —CH₃ | —OC(O)CH₃ | H | H | H |
| 32 | —(CH₂)₂CH₃ | —(CH₂)₂CH₃ | —CH₃ | —OC(O)CH₃ | H | H | H |
| 33 | —(CH₂)₂CH₃ | —CH(CH₃)₂ | —CH₃ | —OC(O)CH₃ | H | H | H |
| 34 | —(CH₂)₂CH₃ | —(CH₂)₃CH₃ | —CH₃ | —OC(O)CH₃ | H | H | H |
| 35 | —(CH₂)₂CH₃ | —CH₂—HC═CH₂ | —CH₃ | —OC(O)CH₃ | H | H | H |
| 36 | —CH(CH₃)₂ | —CH(CH₃)₂ | —CH₃ | —OC(O)CH₃ | H | H | H |
| 37 | —CH(CH₃)₂ | —(CH₂)₃CH₃ | —CH₃ | —OC(O)CH₃ | H | H | H |
| 38 | —CH(CH₃)₂ | —CH₂—HC═CH₂ | —CH₃ | —OC(O)CH₃ | H | H | H |
| 39 | —(CH₂)₃CH₃ | —(CH₂)₃CH₃ | —CH₃ | —OC(O)CH₃ | H | H | H |
| 40 | —(CH₂)₃CH₃ | —CH₂—HC═CH₂ | —CH₃ | —OC(O)CH₃ | H | H | H |
| 41 | —CH₂—HC═CH₂ | —CH₂—HC═CH₂ | —CH₃ | —OC(O)CH₃ | H | H | H |
| 42 | —CH₃ | —CH₃ | —CH₃ | —OC(O)CH₃ | H | H | H |
| 43 | —CH₃ | —CH₂CH₃ | —CH₃ | —OC(O)CH₃ | H | H | H |
| 44 | —CH₃ | —(CH₂)₂CH₃ | —CH₃ | —OC(O)CH₃ | H | H | H |
| 45 | —CH₃ | —CH(CH₃)₂ | —CH₃ | —OC(O)CH₃ | H | H | H |
| 46 | —CH₃ | —(CH₂)₃CH₃ | —CH₃ | —OC(O)CH₃ | H | H | H |
| 47 | —CH₃ | —CH₂—HC═CH₂ | —CH₃ | —OC(O)CH₃ | H | H | H |
| 48 | —CH₂CH₃ | —CH₂CH₃ | —CH₃ | —OC(O)CH₃ | H | H | H |
| 49 | —CH₂CH₃ | —(CH₂)₂CH₃ | —CH₃ | —OC(O)CH₃ | H | H | H |
| 50 | —CH₂CH₃ | —CH(CH₃)₂ | —CH₃ | —OC(O)CH₃ | H | H | H |
| 51 | —CH₂CH₃ | —(CH₂)₃CH₃ | —CH₃ | —OC(O)CH₃ | H | H | H |
| 52 | —CH₂CH₃ | —CH₂—HC═CH₂ | —CH₃ | —OC(O)CH₃ | H | H | H |
| 53 | —(CH₂)₂CH₃ | —(CH₂)₂CH₃ | —CH₃ | —OC(O)CH₃ | H | H | H |
| 54 | —(CH₂)₂CH₃ | —CH(CH₃)₂ | —CH₃ | —OC(O)CH₃ | H | H | H |
| 55 | —(CH₂)₂CH₃ | —(CH₂)₃CH₃ | —CH₃ | —OC(O)CH₃ | H | H | H |
| 56 | —(CH₂)₂CH₃ | —CH₂—HC═CH₂ | —CH₃ | —OC(O)CH₃ | H | H | H |
| 57 | —CH(CH₃)₂ | —CH(CH₃)₂ | —CH₃ | —OC(O)CH₃ | H | H | H |
| 58 | —CH(CH₃)₂ | —(CH₂)₃CH₃ | —CH₃ | —OC(O)CH₃ | H | H | H |
| 59 | —CH(CH₃)₂ | —CH₂—HC═CH₂ | —CH₃ | —OC(O)CH₃ | H | H | H |
| 60 | —(CH₂)₃CH₃ | —(CH₂)₃CH₃ | —CH₃ | —OC(O)CH₃ | H | H | H |
| 61 | —(CH₂)₃CH₃ | —CH₂—HC═CH₂ | —CH₃ | —OC(O)CH₃ | H | H | H |
| 62 | —CH₂—HC═CH₂ | —CH₂—HC═CH₂ | —CH₃ | —OC(O)CH₃ | H | H | H |
| 63 | —CH₃ | —CH₃ | —CH₃ | H | H | H | H |
| 64 | —CH₃ | —CH₂CH₃ | —CH₃ | H | H | H | H |
| 65 | —CH₃ | —(CH₂)₂CH₃ | —CH₃ | H | H | H | H |
| 66 | —CH₃ | —CH(CH₃)₂ | —CH₃ | H | H | H | H |
| 67 | —CH₃ | —(CH₂)₃CH₃ | —CH₃ | H | H | H | H |
| 68 | —CH₃ | —CH₂—HC═CH₂ | —CH₃ | H | H | H | H |
| 69 | —CH₂CH₃ | —CH₂CH₃ | —CH₃ | H | H | H | H |
| 70 | —CH₂CH₃ | —(CH₂)₂CH₃ | —CH₃ | H | H | H | H |
| 71 | —CH₂CH₃ | —CH(CH₃)₂ | —CH₃ | H | H | H | H |
| 72 | —CH₂CH₃ | —(CH₂)₃CH₃ | —CH₃ | H | H | H | H |
| 73 | —CH₂CH₃ | —CH₂—HC═CH₂ | —CH₃ | H | H | H | H |
| 74 | —(CH₂)₂CH₃ | —(CH₂)₂CH₃ | —CH₃ | H | H | H | H |
| 75 | —(CH₂)₂CH₃ | —CH(CH₃)₂ | —CH₃ | H | H | H | H |
| 76 | —(CH₂)₂CH₃ | —(CH₂)₃CH₃ | —CH₃ | H | H | H | H |
| 77 | —(CH₂)₂CH₃ | —CH₂—HC═CH₂ | —CH₃ | H | H | H | H |
| 78 | —CH(CH₃)₂ | —CH(CH₃)₂ | —CH₃ | H | H | H | H |
| 79 | —CH(CH₃)₂ | —(CH₂)₃CH₃ | —CH₃ | H | H | H | H |
| 80 | —CH(CH₃)₂ | —CH₂—HC═CH₂ | —CH₃ | H | H | H | H |
| 81 | —(CH₂)₃CH₃ | —(CH₂)₃CH₃ | —CH₃ | H | H | H | H |
| 82 | —(CH₂)₃CH₃ | —CH₂—HC═CH₂ | —CH₃ | H | H | H | H |
| 83 | —CH₂—HC═CH₂ | —CH₂—HC═CH₂ | —CH₃ | H | H | H | H |
| 84 | —CH₃ | —CH₂CH₃ | —CH₃ | H | —OCH₃ | H | H |
| 85 | —CH₃ | —(CH₂)₂CH₃ | —CH₃ | H | —OCH₃ | H | H |
| 86 | —CH₃ | —CH(CH₃)₂ | —CH₃ | H | —OCH₃ | H | H |
| 87 | —CH₃ | —(CH₂)₃CH₃ | —CH₃ | H | —OCH₃ | H | H |
| 88 | —CH₃ | —CH₂—HC═CH₂ | —CH₃ | H | —OCH₃ | H | H |
| 89 | —CH₂CH₃ | —CH₂CH₃ | —CH₃ | H | —OCH₃ | H | H |
| 90 | —CH₂CH₃ | —(CH₂)₂CH₃ | —CH₃ | H | —OCH₃ | H | H |
| 91 | —CH₂CH₃ | —CH(CH₃)₂ | —CH₃ | H | —OCH₃ | H | H |
| 92 | —CH₂CH₃ | —(CH₂)₃CH₃ | —CH₃ | H | —OCH₃ | H | H |
| 93 | —CH₂CH₃ | —CH₂—HC═CH₂ | —CH₃ | H | —OCH₃ | H | H |
| 94 | —(CH₂)₂CH₃ | —(CH₂)₂CH₃ | —CH₃ | H | —OCH₃ | H | H |
| 95 | —(CH₂)₂CH₃ | —CH(CH₃)₂ | —CH₃ | H | —OCH₃ | H | H |
| 96 | —(CH₂)₂CH₃ | —(CH₂)₃CH₃ | —CH₃ | H | —OCH₃ | H | H |
| 97 | —(CH₂)₂CH₃ | —CH₂—HC═CH₂ | —CH₃ | H | —OCH₃ | H | H |
| 98 | —CH(CH₃)₂ | —CH(CH₃)₂ | —CH₃ | H | —OCH₃ | H | H |
| 99 | —CH(CH₃)₂ | —(CH₂)₃CH₃ | —CH₃ | H | —OCH₃ | H | H |
| 100 | —CH(CH₃)₂ | —CH₂—HC═CH₂ | —CH₃ | H | —OCH₃ | H | H |
| 101 | —(CH₂)₃CH₃ | —(CH₂)₃CH₃ | —CH₃ | H | —OCH₃ | H | H |
| 102 | —(CH₂)₃CH₃ | —CH₂—HC═CH₂ | —CH₃ | H | —OCH₃ | H | H |
| 103 | —CH₂—HC═CH₂ | —CH₂—HC═CH₂ | —CH₃ | H | —OCH₃ | H | H |
| 104 | —CH₃ | —CH₃ | —CH₃ | H | —OCH₃ | H | H |
| 105 | —CH₃ | —CH₂CH₃ | —CH₃ | H | H | F | H |
| 106 | —CH₃ | —(CH₂)₂CH₃ | —CH₃ | H | H | F | H |
| 107 | —CH₃ | —CH(CH₃)₂ | —CH₃ | H | H | F | H |
| 108 | —CH₃ | —(CH₂)₃CH₃ | —CH₃ | H | H | F | H |
| 109 | —CH₃ | —CH₂—HC═CH₂ | —CH₃ | H | H | F | H |
| 110 | —CH₂CH₃ | —CH₂CH₃ | —CH₃ | H | H | F | H |
| 111 | —CH₂CH₃ | —(CH₂)₂CH₃ | —CH₃ | H | H | F | H |
| 112 | —CH₂CH₃ | —CH(CH₃)₂ | —CH₃ | H | H | F | H |
| 113 | —CH₂CH₃ | —(CH₂)₃CH₃ | —CH₃ | H | H | F | H |
| 114 | —CH₂CH₃ | —CH₂—HC═CH₂ | —CH₃ | H | H | F | H |
| 115 | —(CH₂)₂CH₃ | —(CH₂)₂CH₃ | —CH₃ | H | H | F | H |
| 116 | —(CH₂)₂CH₃ | —CH(CH₃)₂ | —CH₃ | H | H | F | H |
| 117 | —(CH₂)₂CH₃ | —(CH₂)₃CH₃ | —CH₃ | H | H | F | H |
| 118 | —(CH₂)₂CH₃ | —CH₂—HC═CH₂ | —CH₃ | H | H | F | H |
| 119 | —CH(CH₃)₂ | —CH(CH₃)₂ | —CH₃ | H | H | F | H |
| 120 | —CH(CH₃)₂ | —(CH₂)₃CH₃ | —CH₃ | H | H | F | H |
| 121 | —CH(CH₃)₂ | —CH₂—HC═CH₂ | —CH₃ | H | H | F | H |
| 122 | —(CH₂)₃CH₃ | —(CH₂)₃CH₃ | —CH₃ | H | H | F | H |
| 123 | —(CH₂)₃CH₃ | —CH₂—HC═CH₂ | —CH₃ | H | H | F | H |
| 124 | —CH₂—HC═CH₂ | —CH₂—HC═CH₂ | —CH₃ | H | H | F | H |
| 125 | —CH₃ | —CH₃ | —CH₃ | H | H | F | H |
| 126 | —CH₃ | —CH₂CH₃ | —CH₃ | H | H | H | —CH₃ |
| 127 | —CH₃ | —(CH₂)₂CH₃ | —CH₃ | H | H | H | —CH₃ |
| 128 | —CH₃ | —CH(CH₃)₂ | —CH₃ | H | H | H | —CH₃ |
| 129 | —CH₃ | —(CH₂)₃CH₃ | —CH₃ | H | H | H | —CH₃ |
| 130 | —CH₃ | —CH₂—HC═CH₂ | —CH₃ | H | H | H | —CH₃ |
| 131 | —CH₂CH₃ | —CH₂CH₃ | —CH₃ | H | H | H | —CH₃ |
| 132 | —CH₂CH₃ | —(CH₂)₂CH₃ | —CH₃ | H | H | H | —CH₃ |
| 133 | —CH₂CH₃ | —CH(CH₃)₂ | —CH₃ | H | H | H | —CH₃ |
| 134 | —CH₂CH₃ | —(CH₂)₃CH₃ | —CH₃ | H | H | H | —CH₃ |
| 135 | —CH₂CH₃ | —CH₂—HC═CH₂ | —CH₃ | H | H | H | —CH₃ |
| 135 | —(CH₂)₂CH₃ | —(CH₂)₂CH₃ | —CH₃ | H | H | H | —CH₃ |
| 137 | —(CH₂)₂CH₃ | —CH(CH₃)₂ | —CH₃ | H | H | H | —CH₃ |
| 138 | —(CH₂)₂CH₃ | —(CH₂)₃CH₃ | —CH₃ | H | H | H | —CH₃ |
| 139 | —(CH₂)₂CH₃ | —CH₂—HC═CH₂ | —CH₃ | H | H | H | —CH₃ |
| 140 | —CH(CH₃)₂ | —CH(CH₃)₂ | —CH₃ | H | H | H | —CH₃ |
| 141 | —CH(CH₃)₂ | —(CH₂)₃CH₃ | —CH₃ | H | H | H | —CH₃ |
| 142 | —CH(CH₃)₂ | —CH₂—HC═CH₂ | —CH₃ | H | H | H | —CH₃ |
| 143 | —(CH₂)₃CH₃ | —(CH₂)₃CH₃ | —CH₃ | H | H | H | —CH₃ |
| 144 | —(CH₂)₃CH₃ | —CH₂—HC═CH₂ | —CH₃ | H | H | H | —CH₃ |
| 145 | —CH₂—HC═CH₂ | —CH₂—HC═CH₂ | —CH₃ | H | H | H | —CH₃ |
| 146 | —CH₃ | —CH₂CH₃ | —CH₃ | H | F | H | H |
| 147 | —CH₃ | —(CH₂)₂CH₃ | —CH₃ | H | F | H | H |
| 148 | —CH₃ | —CH(CH₃)₂ | —CH₃ | H | F | H | H |
| 149 | —CH₃ | —(CH₂)₃CH₃ | —CH₃ | H | F | H | H |
| 150 | —CH₃ | —CH₂—HC═CH₂ | —CH₃ | H | F | H | H |
| 151 | —CH₂CH₃ | —CH₂CH₃ | —CH₃ | H | F | H | H |
| 152 | —CH₂CH₃ | —(CH₂)₂CH₃ | —CH₃ | H | F | H | H |
| 153 | —CH₂CH₃ | —CH(CH₃)₂ | —CH₃ | H | F | H | H |
| 154 | —CH₂CH₃ | —(CH₂)₃CH₃ | —CH₃ | H | F | H | H |
| 155 | —CH₂CH₃ | —CH₂—HC═CH₂ | —CH₃ | H | F | H | H |
| 156 | —(CH₂)₂CH₃ | —(CH₂)₂CH₃ | —CH₃ | H | F | H | H |
| 157 | —(CH₂)₂CH₃ | —CH(CH₃)₂ | —CH₃ | H | F | H | H |
| 158 | —(CH₂)₂CH₃ | —(CH₂)₃CH₃ | —CH₃ | H | F | H | H |
| 159 | —(CH₂)₂CH₃ | —CH₂—HC═CH₂ | —CH₃ | H | F | H | H |
| 160 | —CH(CH₃)₂ | —CH(CH₃)₂ | —CH₃ | H | F | H | H |
| 161 | —CH(CH₃)₂ | —(CH₂)₃CH₃ | —CH₃ | H | F | H | H |
| 162 | —CH(CH₃)₂ | —CH₂—HC═CH₂ | —CH₃ | H | F | H | H |
| 163 | —(CH₂)₃CH₃ | —(CH₂)₃CH₃ | —CH₃ | H | F | H | H |
| 164 | —(CH₂)₃CH₃ | —CH₂—HC═CH₂ | —CH₃ | H | F | H | H |
| 165 | —CH₂—HC═CH₂ | —CH₂—HC═CH₂ | —CH₃ | H | F | H | H |
| 166 | —CH₃ | —CH₃ | —CH₃ | H | F | H | H |
| 167 | —CH₃ | —CH₂CH₃ | —CH₃ | —OC(O)CH₃ | F | H | H |
| 168 | —CH₃ | —(CH₂)₂CH₃ | —CH₃ | —OC(O)CH₃ | F | H | H |
| 169 | —CH₃ | —CH(CH₃)₂ | —CH₃ | —OC(O)CH₃ | F | H | H |
| 170 | —CH₃ | —(CH₂)₃CH₃ | —CH₃ | —OC(O)CH₃ | F | H | H |
| 171 | —CH₃ | —CH₂—HC═CH₂ | —CH₃ | —OC(O)CH₃ | F | H | H |
| 172 | —CH₂CH₃ | —CH₂CH₃ | —CH₃ | —OC(O)CH₃ | F | H | H |
| 173 | —CH₂CH₃ | —(CH₂)₂CH₃ | —CH₃ | —OC(O)CH₃ | F | H | H |
| 174 | —CH₂CH₃ | —CH(CH₃)₂ | —CH₃ | —OC(O)CH₃ | F | H | H |
| 175 | —CH₂CH₃ | —(CH₂)₃CH₃ | —CH₃ | —OC(O)CH₃ | F | H | H |
| 176 | —CH₂CH₃ | —CH₂—HC═CH₂ | —CH₃ | —OC(O)CH₃ | F | H | H |
| 177 | —(CH₂)₂CH₃ | —(CH₂)₂CH₃ | —CH₃ | —OC(O)CH₃ | F | H | H |
| 178 | —(CH₂)₂CH₃ | —CH(CH₃)₂ | —CH₃ | —OC(O)CH₃ | F | H | H |
| 179 | —(CH₂)₂CH₃ | —(CH₂)₃CH₃ | —CH₃ | —OC(O)CH₃ | F | H | H |
| 180 | —(CH₂)₂CH₃ | —CH₂—HC═CH₂ | —CH₃ | —OC(O)CH₃ | F | H | H |
| 181 | —CH(CH₃)₂ | —CH(CH₃)₂ | —CH₃ | —OC(O)CH₃ | F | H | H |
| 182 | —CH(CH₃)₂ | —(CH₂)₃CH₃ | —CH₃ | —OC(O)CH₃ | F | H | H |
| 183 | —CH(CH₃)₂ | —CH₂—HC═CH₂ | —CH₃ | —OC(O)CH₃ | F | H | H |
| 184 | —(CH₂)₃CH₃ | —(CH₂)₃CH₃ | —CH₃ | —OC(O)CH₃ | F | H | H |
| 185 | —(CH₂)₃CH₃ | —CH₂—HC═CH₂ | —CH₃ | —OC(O)CH₃ | F | H | H |
| 186 | —CH₂—HC═CH₂ | —CH₂—HC═CH₂ | —CH₃ | —OC(O)CH₃ | F | H | H |
| 187 | —CH₃ | —CH₃ | —CH₃ | —OC(O)CH₃ | F | F | H |
| 188 | —CH₃ | —CH₂CH₃ | —CH₃ | H | H | F | H |
| 189 | —CH₃ | —(CH₂)₂CH₃ | —CH₃ | H | H | F | H |
| 190 | —CH₃ | —CH(CH₃)₂ | —CH₃ | H | H | F | H |
| 191 | —CH₃ | —(CH₂)₃CH₃ | —CH₃ | H | H | F | H |
| 192 | —CH₃ | —CH₂—HC═CH₂ | —CH₃ | H | H | F | H |
| 193 | —CH₂CH₃ | —CH₂CH₃ | —CH₃ | H | H | F | H |
| 194 | —CH₂CH₃ | —(CH₂)₂CH₃ | —CH₃ | H | H | F | H |
| 195 | —CH₂CH₃ | —CH(CH₃)₂ | —CH₃ | H | H | F | H |
| 196 | —CH₂CH₃ | —(CH₂)₃CH₃ | —CH₃ | H | H | F | H |
| 197 | —CH₂CH₃ | —CH₂—HC═CH₂ | —CH₃ | H | H | F | H |
| 198 | —(CH₂)₂CH₃ | —(CH₂)₂CH₃ | —CH₃ | H | H | F | H |
| 199 | —(CH₂)₂CH₃ | —CH(CH₃)₂ | —CH₃ | H | H | F | H |
| 200 | —(CH₂)₂CH₃ | —(CH₂)₃CH₃ | —CH₃ | H | H | F | H |
| 201 | —(CH₂)₂CH₃ | —CH₂—HC═CH₂ | —CH₃ | H | H | F | H |
| 202 | —CH(CH₃)₂ | —CH(CH₃)₂ | —CH₃ | H | H | F | H |
| 203 | —CH(CH₃)₂ | —(CH₂)₃CH₃ | —CH₃ | H | H | F | H |
| 204 | —CH(CH₃)₂ | —CH₂—HC═CH₂ | —CH₃ | H | H | F | H |
| 205 | —(CH₂)₃CH₃ | —(CH₂)₃CH₃ | —CH₃ | H | H | F | H |
| 206 | —(CH₂)₃CH₃ | —CH₂—HC═CH₂ | —CH₃ | H | H | F | H |
| 207 | —CH₂—HC═CH₂ | —CH₂—HC═CH₂ | —CH₃ | H | H | F | H |
| 208 | —CH₃ | —CH₂CH₃ | —CH₃ | H | H | F | H |
| 209 | —CH₃ | —(CH₂)₂CH₃ | —CH₃ | —OC(O)CH₃ | H | F | H |
| 210 | —CH₃ | —CH(CH₃)₂ | —CH₃ | —OC(O)CH₃ | H | F | H |
| 211 | —CH₃ | —(CH₂)₃CH₃ | —CH₃ | —OC(O)CH₃ | H | F | H |
| 212 | —CH₃ | —CH₂—HC═CH₂ | —CH₃ | —OC(O)CH₃ | H | F | H |
| 213 | —CH₂CH₃ | —CH₂CH₃ | —CH₃ | —OC(O)CH₃ | H | F | H |
| 214 | —CH₂CH₃ | —(CH₂)₂CH₃ | —CH₃ | —OC(O)CH₃ | H | F | H |
| 215 | —CH₂CH₃ | —CH(CH₃)₂ | —CH₃ | —OC(O)CH₃ | H | F | H |
| 216 | —CH₂CH₃ | —(CH₂)₃CH₃ | —CH₃ | —OC(O)CH₃ | H | F | H |
| 217 | —CH₂CH₃ | —CH₂—HC═CH₂ | —CH₃ | —OC(O)CH₃ | H | F | H |
| 218 | —(CH₂)₂CH₃ | —(CH₂)₂CH₃ | —CH₃ | —OC(O)CH₃ | H | F | H |
| 219 | —(CH₂)₂CH₃ | —CH(CH₃)₂ | —CH₃ | —OC(O)CH₃ | H | F | H |
| 220 | —(CH₂)₂CH₃ | —(CH₂)₃CH₃ | —CH₃ | —OC(O)CH₃ | H | F | H |
| 221 | —(CH₂)₂CH₃ | —CH₂—HC═CH₂ | —CH₃ | —OC(O)CH₃ | H | F | H |
| 222 | —CH(CH₃)₂ | —CH(CH₃)₂ | —CH₃ | —OC(O)CH₃ | H | F | H |
| 223 | —CH(CH₃)₂ | —(CH₂)₃CH₃ | —CH₃ | —OC(O)CH₃ | H | F | H |
| 224 | —CH(CH₃)₂ | —CH₂—HC═CH₂ | —CH₃ | —OC(O)CH₃ | H | F | H |
| 225 | —(CH₂)₃CH₃ | —(CH₂)₃CH₃ | —CH₃ | —OC(O)CH₃ | H | F | H |
| 226 | —(CH₂)₃CH₃ | —CH₂—HC═CH₂ | —CH₃ | —OC(O)CH₃ | H | F | H |
| 227 | —CH₂—HC═CH₂ | —CH₂—HC═CH₂ | —CH₃ | —OC(O)CH₃ | H | F | H |
| 228 | —CH₃ | —CH₃ | —CH₃ | —OC(O)CH₃ | H | F | H |
| 229 | —CH₃ | —CH₂CH₃ | allyl | H | H | H | H |
| 230 | —CH₃ | —(CH₂)₂CH₃ | allyl | H | H | H | H |
| 231 | —CH₃ | —CH(CH₃)₂ | allyl | H | H | H | H |
| 232 | —CH₃ | —(CH₂)₃CH₃ | allyl | H | H | H | H |
| 233 | —CH₃ | —CH₂—HC═CH₂ | allyl | H | H | H | H |
| 234 | —CH₂CH₃ | —CH₂CH₃ | allyl | H | H | H | H |
| 235 | —CH₂CH₃ | —(CH₂)₂CH₃ | allyl | H | H | H | H |
| 236 | —CH₂CH₃ | —CH(CH₃)₂ | allyl | H | H | H | H |
| 237 | —CH₂CH₃ | —(CH₂)₃CH₃ | allyl | H | H | H | H |
| 238 | —CH₂CH₃ | —CH₂—HC═CH₂ | allyl | H | H | H | H |
| 239 | —(CH₂)₂CH₃ | —(CH₂)₂CH₃ | allyl | H | H | H | H |
| 240 | —(CH₂)₂CH₃ | —CH(CH₃)₂ | allyl | H | H | H | H |
| 241 | —(CH₂)₂CH₃ | —(CH₂)₃CH₃ | allyl | H | H | H | H |
| 242 | —(CH₂)₂CH₃ | —CH₂—HC═CH₂ | allyl | H | H | H | H |
| 243 | —CH(CH₃)₂ | —CH(CH₃)₂ | allyl | H | H | H | H |
| 244 | —CH(CH₃)₂ | —(CH₂)₃CH₃ | allyl | H | H | H | H |
| 245 | —CH(CH₃)₂ | —CH₂—HC═CH₂ | allyl | H | H | H | H |
| 246 | —(CH₂)₃CH₃ | —(CH₂)₃CH₃ | allyl | H | H | H | H |
| 247 | —(CH₂)₃CH₃ | —CH₂—HC═CH₂ | allyl | H | H | H | H |
| 248 | —CH₂—HC═CH₂ | —CH₂—HC═CH₂ | allyl | H | H | H | H |
| 249 | H | —CH₃ | —CH₃ | H | H | H | H |
| 250 | H | —CH₂CH₃ | —CH₃ | H | H | H | H |
| 251 | H | —(CH₂)₂CH₃ | —CH₃ | H | H | H | H |
| 252 | H | —CH(CH₃)₂ | —CH₃ | H | H | H | H |
| 253 | H | —(CH₂)₃CH₃ | —CH₃ | H | H | H | H |
| 254 | H | —CH₂—HC═CH₂ | —CH₃ | H | H | H | H |
| 255 | H | —CH₃ | —CH₃ | H | F | H | H |
| 256 | H | —CH₂CH₃ | —CH₃ | H | F | H | H |
| 257 | H | —(CH₂)₂CH₃ | —CH₃ | H | F | H | H |
| 258 | H | —CH(CH₃)₂ | —CH₃ | H | F | H | H |
| 259 | H | —(CH₂)₃CH₃ | —CH₃ | H | F | H | H |
| 260 | H | —CH₂—HC═CH₂ | —CH₃ | H | F | H | H |
| 261 | H | —CH₃ | —CH₃ | H | H | F | H |
| 262 | H | —CH₂CH₃ | —CH₃ | H | H | F | H |
| 263 | H | —(CH₂)₂CH₃ | —CH₃ | H | H | F | H |
| 264 | H | —CH(CH₃)₂ | —CH₃ | H | H | F | H |
| 265 | H | —(CH₂)₃CH₃ | —CH₃ | H | H | F | H |
| 266 | H | —CH₂—HC═CH₂ | —CH₃ | H | H | F | H |
| 267 | H | —CH₃ | —CH₃ | —OC(O)CH₃ | H | H | H |
| 268 | H | —CH₂CH₃ | —CH₃ | —OC(O)CH₃ | H | H | H |
| 269 | H | —(CH₂)₂CH₃ | —CH₃ | —OC(O)CH₃ | H | H | H |
| 270 | H | —CH(CH₃)₂ | —CH₃ | —OC(O)CH₃ | H | H | H |
| 271 | H | —(CH₂)₃CH₃ | —CH₃ | —OC(O)CH₃ | H | H | H |
| 272 | H | —CH₂—HC═CH₂ | —CH₃ | —OC(O)CH₃ | H | H | H |
| 273 | H | —CH₃ | —CH₃ | —OC(O)CH₃ | F | H | H |
| 274 | H | —CH₂CH₃ | —CH₃ | —OC(O)CH₃ | F | H | H |
| 275 | H | —(CH₂)₂CH₃ | —CH₃ | —OC(O)CH₃ | F | H | H |
| 276 | H | —CH(CH₃)₂ | —CH₃ | —OC(O)CH₃ | F | H | H |
| 277 | H | —(CH₂)₃CH₃ | —CH₃ | —OC(O)CH₃ | F | H | H |
| 278 | H | —CH₂—HC═CH₂ | —CH₃ | —OC(O)CH₃ | F | H | H |
| 279 | H | —CH₃ | —CH₃ | —OC(O)CH₃ | H | F | H |
| 280 | H | —CH₂CH₃ | —CH₃ | —OC(O)CH₃ | H | F | H |
| 281 | H | —(CH₂)₂CH₃ | —CH₃ | —OC(O)CH₃ | H | F | H |
| 282 | H | —CH(CH₃)₂ | —CH₃ | —OC(O)CH₃ | H | F | H |
| 283 | H | —(CH₂)₃CH₃ | —CH₃ | —OC(O)CH₃ | H | F | H |
| 284 | H | —CH₂—HC═CH₂ | —CH₃ | —OC(O)CH₃ | H | F | H |
| 285 | H | —CH₃ | —CH₃ | H | F | F | H |
| 286 | H | —CH₂CH₃ | —CH₃ | H | F | F | H |
| 287 | H | —(CH₂)₂CH₃ | —CH₃ | H | F | F | H |
| 288 | H | —CH(CH₃)₂ | —CH₃ | H | F | F | H |
| 289 | H | —(CH₂)₃CH₃ | —CH₃ | H | F | F | H |
| 290 | H | —CH₂—HC═CH₂ | —CH₃ | H | F | F | H |
| 291 | H | —CH₃ | —CH₃ | H | H | H | H |
| 292 | H | —CH₂CH₃ | —CH₃ | H | H | H | H |
| 293 | H | —(CH₂)₂CH₃ | —CH₃ | H | H | H | H |
| 294 | H | —CH(CH₃)₂ | —CH₃ | H | H | H | H |
| 295 | H | —(CH₂)₃CH₃ | —CH₃ | H | H | H | H |
| 296 | H | —CH₂—HC═CH₂ | —CH₃ | H | H | H | H |
| 297 | H | —CH₃ | —CH₃ | H | —OCH₃ | H | H |
| 298 | H | —CH₂CH₃ | —CH₃ | H | —OCH₃ | H | H |
| 299 | H | —(CH₂)₂CH₃ | —CH₃ | H | —OCH₃ | H | H |
| 300 | H | —CH(CH₃)₂ | —CH₃ | H | —OCH₃ | H | H |
| 301 | H | —(CH₂)₃CH₃ | —CH₃ | H | —OCH₃ | H | H |
| 302 | H | —CH₂—HC═CH₂ | —CH₃ | H | —OCH₃ | H | H |
| 303 | H | —CH₃ | —CH₃ | H | H | F | H |
| 304 | H | —CH₂CH₃ | —CH₃ | H | H | F | H |
| 305 | H | —(CH₂)₂CH₃ | —CH₃ | H | H | F | H |
| 306 | H | —CH(CH₃)₂ | —CH₃ | H | H | F | H |
| 307 | H | —(CH₂)₃CH₃ | —CH₃ | H | H | F | H |
| 308 | H | —CH₂—HC═CH₂ | —CH₃ | H | H | F | H |
| 309 | H | cyclopropyl | —CH₃ | H | H | H | H |
| 310 | H | cyclopropyl | —CH₃ | H | H | H | H |
| 311 | H | cyclopropyl | —CH₃ | —OH | H | H | H |
| 312 | H | cyclopropyl | —CH₃ | —OC(O)CH₃ | H | H | H |
| 313 | H | cyclopropyl | —CH₃ | H | —OCH₃ | H | H |
| 314 | H | cyclopropyl | —CH₃ | H | H | H | H |
| 315 | H | cyclopropyl | —CH₃ | H | H | H | H |
| 316 | H | cyclopropyl | —CH₃ | —OH | F | H | H |
| 317 | H | cyclopropyl | —CH₃ | —OH | H | H | H |
| 318 | H | cyclopropyl | —CH₃ | —OC(O)CH₃ | H | H | H |
| 319 | H | cyclopropyl | —CH₃ | H | F | H | H |
| 320 | H | cyclopropyl | —CH₃ | H | H | F | H |
| 321 | H | —CD₃ | —CH₃ | H | H | H | H |
| 322 | H | —CD₃ | —CH₃ | —OH | H | H | H |
| 323 | H | —CD₃ | —CH₃ | —OC(O)CH₃ | H | H | H |
| 324 | H | —CD₃ | —CH₃ | H | —OCH₃ | H | H |
| 325 | H | —CD₃ | —CH₃ | H | F | H | H |
| 326 | H | —CD₃ | —CH₃ | —OH | F | H | H |
| 327 | H | —CD₃ | —CH₃ | —OC(O)CH₃ | F | F | H |
| 328 | H | —CD₃ | —CH₃ | H | F | F | H |
| 329 | H | —CD₂CD₃ | H | H | F | H | H |
| 330 | H | —CD₂CD₃ | H | —OH | H | H | H |
| 331 | H | —CD₂CD₃ | H | —OC(O)CH₃ | H | H | H |
| 332 | H | —CD₂CD₃ | H | H | F | F | H |
| 333 | H | —CD₂CD₃ | H | H | H | F | H |
| 334 | H | —CD₂CD₃ | H | —OH | F | H | H |
| 335 | H | —CD₂CD₃ | H | —OC(O)CH₃ | F | H | H |
| 336 | H | —CD₂CD₃ | H | H | —OCH₃ | F | H |

Other exemplary compounds include compounds of Formula II(b) set forth below in Table 2:

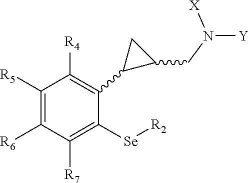

TABLE 2

| Ref. | X | Y | R₂ | R₄ | R₅ | R₆ | R₇ |
|---|---|---|---|---|---|---|---|
| 1 | —CH₃ | —CH₃ | —CH₃ | —OH | H | H | H |
| 2 | —CH₃ | —CH₂CH₃ | —CH₃ | —OH | H | H | H |
| 3 | —CH₃ | —(CH₂)₂CH₃ | —CH₃ | —OH | H | H | H |
| 4 | —CH₃ | —CH(CH₃)₂ | —CH₃ | —OH | H | H | H |
| 5 | —CH₃ | —(CH₂)₃CH₃ | —CH₃ | —OH | H | H | H |
| 6 | —CH₃ | —CH₂—HC═CH₂ | —CH₃ | —OH | H | H | H |
| 7 | —CH₂CH₃ | —CH₂CH₃ | —CH₃ | —OH | H | H | H |
| 8 | —CH₂CH₃ | —(CH₂)₂CH₃ | —CH₃ | —OH | H | H | H |
| 9 | —CH₂CH₃ | —CH(CH₃)₂ | —CH₃ | —OH | H | H | H |
| 10 | —CH₂CH₃ | —(CH₂)₃CH₃ | —CH₃ | —OH | H | H | H |
| 11 | —CH₂CH₃ | —CH₂—HC═CH₂ | —CH₃ | —OH | H | H | H |
| 12 | —(CH₂)₂CH₃ | —(CH₂)₂CH₃ | —CH₃ | —OH | H | H | H |
| 13 | —(CH₂)₂CH₃ | —CH(CH₃)₂ | —CH₃ | —OH | H | H | H |
| 14 | —(CH₂)₂CH₃ | —(CH₂)₃CH₃ | —CH₃ | —OH | H | H | H |
| 15 | —(CH₂)₂CH₃ | —CH₂—HC═CH₂ | —CH₃ | —OH | H | H | H |
| 16 | —CH(CH₃)₂ | —CH(CH₃)₂ | —CH₃ | —OH | H | H | H |
| 17 | —CH(CH₃)₂ | —(CH₂)₃CH₃ | —CH₃ | —OH | H | H | H |
| 18 | —CH(CH₃)₂ | —CH₂—HC═CH₂ | —CH₃ | —OH | H | H | H |
| 19 | —(CH₂)₃CH₃ | —(CH₂)₃CH₃ | —CH₃ | —OH | H | H | H |
| 20 | —(CH₂)₃CH₃ | —CH₂—HC═CH₂ | —CH₃ | —OH | H | H | H |
| 21 | —CH₂—HC═CH₂ | —CH₂—HC═CH₂ | —CH₃ | —OH | H | H | H |
| 22 | —CH₃ | —CH₂CH₃ | —CH₃ | —OC(O)CH₃ | H | H | H |
| 23 | —CH₃ | —(CH₂)₂CH₃ | —CH₃ | —OC(O)CH₃ | H | H | H |
| 24 | —CH₃ | —CH(CH₃)₂ | —CH₃ | —OC(O)CH₃ | H | H | H |
| 25 | —CH₃ | —(CH₂)₃CH₃ | —CH₃ | —OC(O)CH₃ | H | H | H |
| 26 | —CH₃ | —CH₂—HC═CH₂ | —CH₃ | —OC(O)CH₃ | H | H | H |
| 27 | —CH₂CH₃ | —CH₂CH₃ | —CH₃ | —OC(O)CH₃ | H | H | H |
| 28 | —CH₂CH₃ | —(CH₂)₂CH₃ | —CH₃ | —OC(O)CH₃ | H | H | H |
| 29 | —CH₂CH₃ | —CH(CH₃)₂ | —CH₃ | —OC(O)CH₃ | H | H | H |
| 30 | —CH₂CH₃ | —(CH₂)₃CH₃ | —CH₃ | —OC(O)CH₃ | H | H | H |
| 31 | —CH₂CH₃ | —CH₂—HC═CH₂ | —CH₃ | —OC(O)CH₃ | H | H | H |
| 32 | —(CH₂)₂CH₃ | —(CH₂)₂CH₃ | —CH₃ | —OC(O)CH₃ | H | H | H |
| 33 | —(CH₂)₂CH₃ | —CH(CH₃)₂ | —CH₃ | —OC(O)CH₃ | H | H | H |
| 34 | —(CH₂)₂CH₃ | —(CH₂)₃CH₃ | —CH₃ | —OC(O)CH₃ | H | H | H |
| 35 | —(CH₂)₂CH₃ | —CH₂—HC═CH₂ | —CH₃ | —OC(O)CH₃ | H | H | H |
| 36 | —CH(CH₃)₂ | —CH(CH₃)₂ | —CH₃ | —OC(O)CH₃ | H | H | H |
| 37 | —CH(CH₃)₂ | —(CH₂)₃CH₃ | —CH₃ | —OC(O)CH₃ | H | H | H |
| 38 | —CH(CH₃)₂ | —CH₂—HC═CH₂ | —CH₃ | —OC(O)CH₃ | H | H | H |
| 39 | —(CH₂)₃CH₃ | —(CH₂)₃CH₃ | —CH₃ | —OC(O)CH₃ | H | H | H |
| 40 | —(CH₂)₃CH₃ | —CH₂—HC═CH₂ | —CH₃ | —OC(O)CH₃ | H | H | H |
| 41 | —CH₂—HC═CH₂ | —CH₂—HC═CH₂ | —CH₃ | —OC(O)CH₃ | H | H | H |
| 42 | —CH₃ | —CH₃ | —CH₃ | —OC(O)CH₃ | H | H | H |
| 43 | —CH₃ | —CH₂CH₃ | —CH₃ | —OC(O)CH₃ | H | H | H |
| 44 | —CH₃ | —(CH₂)₂CH₃ | —CH₃ | —OC(O)CH₃ | H | H | H |
| 45 | —CH₃ | —CH(CH₃)₂ | —CH₃ | —OC(O)CH₃ | H | H | H |
| 46 | —CH₃ | —(CH₂)₃CH₃ | —CH₃ | —OC(O)CH₃ | H | H | H |
| 47 | —CH₃ | —CH₂—HC═CH₂ | —CH₃ | —OC(O)CH₃ | H | H | H |
| 48 | —CH₂CH₃ | —CH₂CH₃ | —CH₃ | —OC(O)CH₃ | H | H | H |
| 49 | —CH₂CH₃ | —(CH₂)₂CH₃ | —CH₃ | —OC(O)CH₃ | H | H | H |
| 50 | —CH₂CH₃ | —CH(CH₃)₂ | —CH₃ | —OC(O)CH₃ | H | H | H |
| 51 | —CH₂CH₃ | —(CH₂)₃CH₃ | —CH₃ | —OC(O)CH₃ | H | H | H |
| 52 | —CH₂CH₃ | —CH₂—HC═CH₂ | —CH₃ | —OC(O)CH₃ | H | H | H |
| 53 | —(CH₂)₂CH₃ | —(CH₂)₂CH₃ | —CH₃ | —OC(O)CH₃ | H | H | H |
| 54 | —(CH₂)₂CH₃ | —CH(CH₃)₂ | —CH₃ | —OC(O)CH₃ | H | H | H |
| 55 | —(CH₂)₂CH₃ | —(CH₂)₃CH₃ | —CH₃ | —OC(O)CH₃ | H | H | H |
| 56 | —(CH₂)₂CH₃ | —CH₂—HC═CH₂ | —CH₃ | —OC(O)CH₃ | H | H | H |
| 57 | —CH(CH₃)₂ | —CH(CH₃)₂ | —CH₃ | —OC(O)CH₃ | H | H | H |
| 58 | —CH(CH₃)₂ | —(CH₂)₃CH₃ | —CH₃ | —OC(O)CH₃ | H | H | H |
| 59 | —CH(CH₃)₂ | —CH₂—HC═CH₂ | —CH₃ | —OC(O)CH₃ | H | H | H |
| 60 | —(CH₂)₃CH₃ | —(CH₂)₃CH₃ | —CH₃ | —OC(O)CH₃ | H | H | H |
| 61 | —(CH₂)₃CH₃ | —CH₂—HC═CH₂ | —CH₃ | —OC(O)CH₃ | H | H | H |
| 62 | —CH₂—HC═CH₂ | —CH₂—HC═CH₂ | —CH₃ | —OC(O)CH₃ | H | H | H |
| 63 | —CH₃ | —CH₃ | —CH₃ | H | H | H | H |
| 64 | —CH₃ | —CH₂CH₃ | —CH₃ | H | H | H | H |
| 65 | —CH₃ | —(CH₂)₂CH₃ | —CH₃ | H | H | H | H |
| 66 | —CH₃ | —CH(CH₃)₂ | —CH₃ | H | H | H | H |
| 67 | —CH₃ | —(CH₂)₃CH₃ | —CH₃ | H | H | H | H |
| 68 | —CH₃ | —CH₂—HC═CH₂ | —CH₃ | H | H | H | H |
| 69 | —CH₂CH₃ | —CH₂CH₃ | —CH₃ | H | H | H | H |
| 70 | —CH₂CH₃ | —(CH₂)₂CH₃ | —CH₃ | H | H | H | H |
| 71 | —CH₂CH₃ | —CH(CH₃)₂ | —CH₃ | H | H | H | H |
| 72 | —CH₂CH₃ | —(CH₂)₃CH₃ | —CH₃ | H | H | H | H |
| 73 | —CH₂CH₃ | —CH₂—HC═CH₂ | —CH₃ | H | H | H | H |
| 74 | —(CH₂)₂CH₃ | —(CH₂)₂CH₃ | —CH₃ | H | H | H | H |
| 75 | —(CH₂)₂CH₃ | —CH(CH₃)₂ | —CH₃ | H | H | H | H |
| 76 | —(CH₂)₂CH₃ | —(CH₂)₃CH₃ | —CH₃ | H | H | H | H |
| 77 | —(CH₂)₂CH₃ | —CH₂—HC═CH₂ | —CH₃ | H | H | H | H |
| 78 | —CH(CH₃)₂ | —CH(CH₃)₂ | —CH₃ | H | H | H | H |
| 79 | —CH(CH₃)₂ | —(CH₂)₃CH₃ | —CH₃ | H | H | H | H |
| 80 | —CH(CH₃)₂ | —CH₂—HC═CH₂ | —CH₃ | H | H | H | H |
| 81 | —(CH₂)₃CH₃ | —(CH₂)₃CH₃ | —CH₃ | H | H | H | H |
| 82 | —(CH₂)₃CH₃ | —CH₂—HC═CH₂ | —CH₃ | H | H | H | H |
| 83 | —CH₂—HC═CH₂ | —CH₂—HC═CH₂ | —CH₃ | H | H | H | H |
| 84 | —CH₃ | —CH₂CH₃ | —CH₃ | H | —OCH₃ | H | H |
| 85 | —CH₃ | —(CH₂)₂CH₃ | —CH₃ | H | —OCH₃ | H | H |
| 86 | —CH₃ | —CH(CH₃)₂ | —CH₃ | H | —OCH₃ | H | H |
| 87 | —CH₃ | —(CH₂)₃CH₃ | —CH₃ | H | —OCH₃ | H | H |
| 88 | —CH₃ | —CH₂—HC═CH₂ | —CH₃ | H | —OCH₃ | H | H |
| 89 | —CH₂CH₃ | —CH₂CH₃ | —CH₃ | H | —OCH₃ | H | H |
| 90 | —CH₂CH₃ | —(CH₂)₂CH₃ | —CH₃ | H | —OCH₃ | H | H |
| 91 | —CH₂CH₃ | —CH(CH₃)₂ | —CH₃ | H | —OCH₃ | H | H |
| 92 | —CH₂CH₃ | —(CH₂)₃CH₃ | —CH₃ | H | —OCH₃ | H | H |
| 93 | —CH₂CH₃ | —CH₂—HC═CH₂ | —CH₃ | H | —OCH₃ | H | H |
| 94 | —(CH₂)₂CH₃ | —(CH₂)₂CH₃ | —CH₃ | H | —OCH₃ | H | H |
| 95 | —(CH₂)₂CH₃ | —CH(CH₃)₂ | —CH₃ | H | —OCH₃ | H | H |
| 96 | —(CH₂)₂CH₃ | —(CH₂)₃CH₃ | —CH₃ | H | —OCH₃ | H | H |
| 97 | —(CH₂)₂CH₃ | —CH₂—HC═CH₂ | —CH₃ | H | —OCH₃ | H | H |
| 98 | —CH(CH₃)₂ | —CH(CH₃)₂ | —CH₃ | H | —OCH₃ | H | H |
| 99 | —CH(CH₃)₂ | —(CH₂)₃CH₃ | —CH₃ | H | —OCH₃ | H | H |
| 100 | —CH(CH₃)₂ | —CH₂—HC═CH₂ | —CH₃ | H | —OCH₃ | H | H |
| 101 | —(CH₂)₃CH₃ | —(CH₂)₃CH₃ | —CH₃ | H | —OCH₃ | H | H |
| 102 | —(CH₂)₃CH₃ | —CH₂—HC═CH₂ | —CH₃ | H | —OCH₃ | H | H |
| 103 | —CH₂—HC═CH₂ | —CH₂—HC═CH₂ | —CH₃ | H | —OCH₃ | H | H |
| 104 | —CH₃ | —CH₃ | —CH₃ | H | —OCH₃ | H | H |
| 105 | —CH₃ | —CH₂CH₃ | —CH₃ | H | H | F | H |
| 106 | —CH₃ | —(CH₂)₂CH₃ | —CH₃ | H | H | F | H |
| 107 | —CH₃ | —CH(CH₃)₂ | —CH₃ | H | H | F | H |
| 108 | —CH₃ | —(CH₂)₃CH₃ | —CH₃ | H | H | F | H |
| 109 | —CH₃ | —CH₂—HC═CH₂ | —CH₃ | H | H | F | H |
| 110 | —CH₂CH₃ | —CH₂CH₃ | —CH₃ | H | H | F | H |
| 111 | —CH₂CH₃ | —(CH₂)₂CH₃ | —CH₃ | H | H | F | H |
| 112 | —CH₂CH₃ | —CH(CH₃)₂ | —CH₃ | H | H | F | H |
| 113 | —CH₂CH₃ | —(CH₂)₃CH₃ | —CH₃ | H | H | F | H |
| 114 | —CH₂CH₃ | —CH₂—HC═CH₂ | —CH₃ | H | H | F | H |
| 115 | —(CH₂)₂CH₃ | —(CH₂)₂CH₃ | —CH₃ | H | H | F | H |
| 116 | —(CH₂)₂CH₃ | —CH(CH₃)₂ | —CH₃ | H | H | F | H |
| 117 | —(CH₂)₂CH₃ | —(CH₂)₃CH₃ | —CH₃ | H | H | F | H |
| 118 | —(CH₂)₂CH₃ | —CH₂—HC═CH₂ | —CH₃ | H | H | F | H |
| 119 | —CH(CH₃)₂ | —CH(CH₃)₂ | —CH₃ | H | H | F | H |
| 120 | —CH(CH₃)₂ | —(CH₂)₃CH₃ | —CH₃ | H | H | F | H |
| 121 | —CH(CH₃)₂ | —CH₂—HC═CH₂ | —CH₃ | H | H | F | H |
| 122 | —(CH₂)₃CH₃ | —(CH₂)₃CH₃ | —CH₃ | H | H | F | H |
| 123 | —(CH₂)₃CH₃ | —CH₂—HC═CH₂ | —CH₃ | H | H | F | H |
| 124 | —CH₂—HC═CH₂ | —CH₂—HC═CH₂ | —CH₃ | H | H | F | H |
| 125 | —CH₃ | —CH₃ | —CH₃ | H | H | F | H |
| 126 | —CH₃ | —CH₂CH₃ | —CH₃ | H | H | H | —CH₃ |
| 127 | —CH₃ | —(CH₂)₂CH₃ | —CH₃ | H | H | H | —CH₃ |
| 128 | —CH₃ | —CH(CH₃)₂ | —CH₃ | H | H | H | —CH₃ |
| 129 | —CH₃ | —(CH₂)₃CH₃ | —CH₃ | H | H | H | —CH₃ |
| 130 | —CH₃ | —CH₂—HC═CH₂ | —CH₃ | H | H | H | —CH₃ |
| 131 | —CH₂CH₃ | —CH₂CH₃ | —CH₃ | H | H | H | —CH₃ |
| 132 | —CH₂CH₃ | —(CH₂)₂CH₃ | —CH₃ | H | H | H | —CH₃ |
| 133 | —CH₂CH₃ | —CH(CH₃)₂ | —CH₃ | H | H | H | —CH₃ |
| 134 | —CH₂CH₃ | —(CH₂)₃CH₃ | —CH₃ | H | H | H | —CH₃ |
| 135 | —CH₂CH₃ | —CH₂—HC═CH₂ | —CH₃ | H | H | H | —CH₃ |
| 135 | —(CH₂)₂CH₃ | —(CH₂)₂CH₃ | —CH₃ | H | H | H | —CH₃ |
| 137 | —(CH₂)₂CH₃ | —CH(CH₃)₂ | —CH₃ | H | H | H | —CH₃ |
| 138 | —(CH₂)₂CH₃ | —(CH₂)₃CH₃ | —CH₃ | H | H | H | —CH₃ |
| 139 | —(CH₂)₂CH₃ | —CH₂—HC═CH₂ | —CH₃ | H | H | H | —CH₃ |
| 140 | —CH(CH₃)₂ | —CH(CH₃)₂ | —CH₃ | H | H | H | —CH₃ |
| 141 | —CH(CH₃)₂ | —(CH₂)₃CH₃ | —CH₃ | H | H | H | —CH₃ |
| 142 | —CH(CH₃)₂ | —CH₂—HC═CH₂ | —CH₃ | H | H | H | —CH₃ |
| 143 | —(CH₂)₃CH₃ | —(CH₂)₃CH₃ | —CH₃ | H | H | H | —CH₃ |
| 144 | —(CH₂)₃CH₃ | —CH₂—HC═CH₂ | —CH₃ | H | H | H | —CH₃ |
| 145 | —CH₂—HC═CH₂ | —CH₂—HC═CH₂ | —CH₃ | H | H | H | —CH₃ |
| 146 | —CH₃ | —CH₂CH₃ | —CH₃ | H | F | H | H |
| 147 | —CH₃ | —(CH₂)₂CH₃ | —CH₃ | H | F | H | H |
| 148 | —CH₃ | —CH(CH₃)₂ | —CH₃ | H | F | H | H |
| 149 | —CH₃ | —(CH₂)₃CH₃ | —CH₃ | H | F | H | H |
| 150 | —CH₃ | —CH₂—HC═CH₂ | —CH₃ | H | F | H | H |
| 151 | —CH₂CH₃ | —CH₂CH₃ | —CH₃ | H | F | H | H |
| 152 | —CH₂CH₃ | —(CH₂)₂CH₃ | —CH₃ | H | F | H | H |
| 153 | —CH₂CH₃ | —CH(CH₃)₂ | —CH₃ | H | F | H | H |
| 154 | —CH₂CH₃ | —(CH₂)₃CH₃ | —CH₃ | H | F | H | H |
| 155 | —CH₂CH₃ | —CH₂—HC═CH₂ | —CH₃ | H | F | H | H |
| 156 | —(CH₂)₂CH₃ | —(CH₂)₂CH₃ | —CH₃ | H | F | H | H |
| 157 | —(CH₂)₂CH₃ | —CH(CH₃)₂ | —CH₃ | H | F | H | H |
| 158 | —(CH₂)₂CH₃ | —(CH₂)₃CH₃ | —CH₃ | H | F | H | H |
| 159 | —(CH₂)₂CH₃ | —CH₂—HC═CH₂ | —CH₃ | H | F | H | H |
| 160 | —CH(CH₃)₂ | —CH(CH₃)₂ | —CH₃ | H | F | H | H |
| 161 | —CH(CH₃)₂ | —(CH₂)₃CH₃ | —CH₃ | H | F | H | H |
| 162 | —CH(CH₃)₂ | —CH₂—HC═CH₂ | —CH₃ | H | F | H | H |
| 163 | —(CH₂)₃CH₃ | —(CH₂)₃CH₃ | —CH₃ | H | F | H | H |
| 164 | —(CH₂)₃CH₃ | —CH₂—HC═CH₂ | —CH₃ | H | F | H | H |
| 165 | —CH₂—HC═CH₂ | —CH₂—HC═CH₂ | —CH₃ | H | F | H | H |
| 166 | —CH₃ | —CH₃ | —CH₃ | H | F | H | H |
| 167 | —CH₃ | —CH₂CH₃ | —CH₃ | —OC(O)CH₃ | F | H | H |
| 168 | —CH₃ | —(CH₂)₂CH₃ | —CH₃ | —OC(O)CH₃ | F | H | H |
| 169 | —CH₃ | —CH(CH₃)₂ | —CH₃ | —OC(O)CH₃ | F | H | H |
| 170 | —CH₃ | —(CH₂)₃CH₃ | —CH₃ | —OC(O)CH₃ | F | H | H |
| 171 | —CH₃ | —CH₂—HC═CH₂ | —CH₃ | —OC(O)CH₃ | F | H | H |
| 172 | —CH₂CH₃ | —CH₂CH₃ | —CH₃ | —OC(O)CH₃ | F | H | H |
| 173 | —CH₂CH₃ | —(CH₂)₂CH₃ | —CH₃ | —OC(O)CH₃ | F | H | H |
| 174 | —CH₂CH₃ | —CH(CH₃)₂ | —CH₃ | —OC(O)CH₃ | F | H | H |
| 175 | —CH₂CH₃ | —(CH₂)₃CH₃ | —CH₃ | —OC(O)CH₃ | F | H | H |
| 176 | —CH₂CH₃ | —CH₂—HC═CH₂ | —CH₃ | —OC(O)CH₃ | F | H | H |
| 177 | —(CH₂)₂CH₃ | —(CH₂)₂CH₃ | —CH₃ | —OC(O)CH₃ | F | H | H |
| 178 | —(CH₂)₂CH₃ | —CH(CH₃)₂ | —CH₃ | —OC(O)CH₃ | F | H | H |
| 179 | —(CH₂)₂CH₃ | —(CH₂)₃CH₃ | —CH₃ | —OC(O)CH₃ | F | H | H |
| 180 | —(CH₂)₂CH₃ | —CH₂—HC═CH₂ | —CH₃ | —OC(O)CH₃ | F | H | H |
| 181 | —CH(CH₃)₂ | —CH(CH₃)₂ | —CH₃ | —OC(O)CH₃ | F | H | H |
| 182 | —CH(CH₃)₂ | —(CH₂)₃CH₃ | —CH₃ | —OC(O)CH₃ | F | H | H |
| 183 | —CH(CH₃)₂ | —CH₂—HC═CH₂ | —CH₃ | —OC(O)CH₃ | F | H | H |
| 184 | —(CH₂)₃CH₃ | —(CH₂)₃CH₃ | —CH₃ | —OC(O)CH₃ | F | H | H |
| 185 | —(CH₂)₃CH₃ | —CH₂—HC═CH₂ | —CH₃ | —OC(O)CH₃ | F | H | H |
| 186 | —CH₂—HC═CH₂ | —CH₂—HC═CH₂ | —CH₃ | —OC(O)CH₃ | F | H | H |
| 187 | —CH₃ | —CH₃ | —CH₃ | —OC(O)CH₃ | F | F | H |
| 188 | —CH₃ | —CH₂CH₃ | —CH₃ | H | H | F | H |
| 189 | —CH₃ | —(CH₂)₂CH₃ | —CH₃ | H | H | F | H |
| 190 | —CH₃ | —CH(CH₃)₂ | —CH₃ | H | H | F | H |
| 191 | —CH₃ | —(CH₂)₃CH₃ | —CH₃ | H | H | F | H |
| 192 | —CH₃ | —CH₂—HC═CH₂ | —CH₃ | H | H | F | H |
| 193 | —CH₂CH₃ | —CH₂CH₃ | —CH₃ | H | H | F | H |
| 194 | —CH₂CH₃ | —(CH₂)₂CH₃ | —CH₃ | H | H | F | H |
| 195 | —CH₂CH₃ | —CH(CH₃)₂ | —CH₃ | H | H | F | H |
| 196 | —CH₂CH₃ | —(CH₂)₃CH₃ | —CH₃ | H | H | F | H |
| 197 | —CH₂CH₃ | —CH₂—HC═CH₂ | —CH₃ | H | H | F | H |
| 198 | —(CH₂)₂CH₃ | —(CH₂)₂CH₃ | —CH₃ | H | H | F | H |
| 199 | —(CH₂)₂CH₃ | —CH(CH₃)₂ | —CH₃ | H | H | F | H |
| 200 | —(CH₂)₂CH₃ | —(CH₂)₃CH₃ | —CH₃ | H | H | F | H |
| 201 | —(CH₂)₂CH₃ | —CH₂—HC═CH₂ | —CH₃ | H | H | F | H |
| 202 | —CH(CH₃)₂ | —CH(CH₃)₂ | —CH₃ | H | H | F | H |
| 203 | —CH(CH₃)₂ | —(CH₂)₃CH₃ | —CH₃ | H | H | F | H |
| 204 | —CH(CH₃)₂ | —CH₂—HC═CH₂ | —CH₃ | H | H | F | H |
| 205 | —(CH₂)₃CH₃ | —(CH₂)₃CH₃ | —CH₃ | H | H | F | H |
| 206 | —(CH₂)₃CH₃ | —CH₂—HC═CH₂ | —CH₃ | H | H | F | H |
| 207 | —CH₂—HC═CH₂ | —CH₂—HC═CH₂ | —CH₃ | H | H | F | H |
| 208 | —CH₃ | —CH₂CH₃ | —CH₃ | H | H | F | H |
| 209 | —CH₃ | —(CH₂)₂CH₃ | —CH₃ | —OC(O)CH₃ | H | F | H |
| 210 | —CH₃ | —CH(CH₃)₂ | —CH₃ | —OC(O)CH₃ | H | F | H |
| 211 | —CH₃ | —(CH₂)₃CH₃ | —CH₃ | —OC(O)CH₃ | H | F | H |
| 212 | —CH₃ | —CH₂—HC═CH₂ | —CH₃ | —OC(O)CH₃ | H | F | H |
| 213 | —CH₂CH₃ | —CH₂CH₃ | —CH₃ | —OC(O)CH₃ | H | F | H |
| 214 | —CH₂CH₃ | —(CH₂)₂CH₃ | —CH₃ | —OC(O)CH₃ | H | F | H |
| 215 | —CH₂CH₃ | —CH(CH₃)₂ | —CH₃ | —OC(O)CH₃ | H | F | H |
| 216 | —CH₂CH₃ | —(CH₂)₃CH₃ | —CH₃ | —OC(O)CH₃ | H | F | H |
| 217 | —CH₂CH₃ | —CH₂—HC═CH₂ | —CH₃ | —OC(O)CH₃ | H | F | H |
| 218 | —(CH₂)₂CH₃ | —(CH₂)₂CH₃ | —CH₃ | —OC(O)CH₃ | H | F | H |
| 219 | —(CH₂)₂CH₃ | —CH(CH₃)₂ | —CH₃ | —OC(O)CH₃ | H | F | H |
| 220 | —(CH₂)₂CH₃ | —(CH₂)₃CH₃ | —CH₃ | —OC(O)CH₃ | H | F | H |
| 221 | —(CH₂)₂CH₃ | —CH₂—HC═CH₂ | —CH₃ | —OC(O)CH₃ | H | F | H |
| 222 | —CH(CH₃)₂ | —CH(CH₃)₂ | —CH₃ | —OC(O)CH₃ | H | F | H |
| 223 | —CH(CH₃)₂ | —(CH₂)₃CH₃ | —CH₃ | —OC(O)CH₃ | H | F | H |
| 224 | —CH(CH₃)₂ | —CH₂—HC═CH₂ | —CH₃ | —OC(O)CH₃ | H | F | H |
| 225 | —(CH₂)₃CH₃ | —(CH₂)₃CH₃ | —CH₃ | —OC(O)CH₃ | H | F | H |
| 226 | —(CH₂)₃CH₃ | —CH₂—HC═CH₂ | —CH₃ | —OC(O)CH₃ | H | F | H |
| 227 | —CH₂—HC═CH₂ | —CH₂—HC═CH₂ | —CH₃ | —OC(O)CH₃ | H | F | H |
| 228 | —CH₃ | —CH₃ | —CH₃ | —OC(O)CH₃ | H | F | H |
| 229 | —CH₃ | —CH₂CH₃ | allyl | H | H | H | H |
| 230 | —CH₃ | —(CH₂)₂CH₃ | allyl | H | H | H | H |
| 231 | —CH₃ | —CH(CH₃)₂ | allyl | H | H | H | H |
| 232 | —CH₃ | —(CH₂)₃CH₃ | allyl | H | H | H | H |
| 233 | —CH₃ | —CH₂—HC═CH₂ | allyl | H | H | H | H |
| 234 | —CH₂CH₃ | —CH₂CH₃ | allyl | H | H | H | H |
| 235 | —CH₂CH₃ | —(CH₂)₂CH₃ | allyl | H | H | H | H |
| 236 | —CH₂CH₃ | —CH(CH₃)₂ | allyl | H | H | H | H |
| 237 | —CH₂CH₃ | —(CH₂)₃CH₃ | allyl | H | H | H | H |
| 238 | —CH₂CH₃ | —CH₂—HC═CH₂ | allyl | H | H | H | H |
| 239 | —(CH₂)₂CH₃ | —(CH₂)₂CH₃ | allyl | H | H | H | H |
| 240 | —(CH₂)₂CH₃ | —CH(CH₃)₂ | allyl | H | H | H | H |
| 241 | —(CH₂)₂CH₃ | —(CH₂)₃CH₃ | allyl | H | H | H | H |
| 242 | —(CH₂)₂CH₃ | —CH₂—HC═CH₂ | allyl | H | H | H | H |
| 243 | —CH(CH₃)₂ | —CH(CH₃)₂ | allyl | H | H | H | H |
| 244 | —CH(CH₃)₂ | —(CH₂)₃CH₃ | allyl | H | H | H | H |
| 245 | —CH(CH₃)₂ | —CH₂—HC═CH₂ | allyl | H | H | H | H |
| 246 | —(CH₂)₃CH₃ | —(CH₂)₃CH₃ | allyl | H | H | H | H |
| 247 | —(CH₂)₃CH₃ | —CH₂—HC═CH₂ | allyl | H | H | H | H |
| 248 | —CH₂—HC═CH₂ | —CH₂—HC═CH₂ | allyl | H | H | H | H |
| 249 | H | —CH₃ | —CH₃ | H | H | H | H |
| 250 | H | —CH₂CH₃ | —CH₃ | H | H | H | H |
| 251 | H | —(CH₂)₂CH₃ | —CH₃ | H | H | H | H |
| 252 | H | —CH(CH₃)₂ | —CH₃ | H | H | H | H |
| 253 | H | —(CH₂)₃CH₃ | —CH₃ | H | H | H | H |
| 254 | H | —CH₂—HC═CH₂ | —CH₃ | H | H | H | H |
| 255 | H | —CH₃ | —CH₃ | H | F | H | H |
| 256 | H | —CH₂CH₃ | —CH₃ | H | F | H | H |
| 257 | H | —(CH₂)₂CH₃ | —CH₃ | H | F | H | H |
| 258 | H | —CH(CH₃)₂ | —CH₃ | H | F | H | H |
| 259 | H | —(CH₂)₃CH₃ | —CH₃ | H | F | H | H |
| 260 | H | —CH₂—HC═CH₂ | —CH₃ | H | F | H | H |
| 261 | H | —CH₃ | —CH₃ | H | H | F | H |
| 262 | H | —CH₂CH₃ | —CH₃ | H | H | F | H |
| 263 | H | —(CH₂)₂CH₃ | —CH₃ | H | H | F | H |
| 264 | H | —CH(CH₃)₂ | —CH₃ | H | H | F | H |
| 265 | H | —(CH₂)₃CH₃ | —CH₃ | H | H | F | H |
| 266 | H | —CH₂—HC═CH₂ | —CH₃ | H | H | F | H |
| 267 | H | —CH₃ | —CH₃ | —OC(O)CH₃ | H | H | H |
| 268 | H | —CH₂CH₃ | —CH₃ | —OC(O)CH₃ | H | H | H |
| 269 | H | —(CH₂)₂CH₃ | —CH₃ | —OC(O)CH₃ | H | H | H |
| 270 | H | —CH(CH₃)₂ | —CH₃ | —OC(O)CH₃ | H | H | H |
| 271 | H | —(CH₂)₃CH₃ | —CH₃ | —OC(O)CH₃ | H | H | H |
| 272 | H | —CH₂—HC═CH₂ | —CH₃ | —OC(O)CH₃ | H | H | H |
| 273 | H | —CH₃ | —CH₃ | —OC(O)CH₃ | F | H | H |
| 274 | H | —CH₂CH₃ | —CH₃ | —OC(O)CH₃ | F | H | H |
| 275 | H | —(CH₂)₂CH₃ | —CH₃ | —OC(O)CH₃ | F | H | H |
| 276 | H | —CH(CH₃)₂ | —CH₃ | —OC(O)CH₃ | F | H | H |
| 277 | H | —(CH₂)₃CH₃ | —CH₃ | —OC(O)CH₃ | F | H | H |
| 278 | H | —CH₂—HC═CH₂ | —CH₃ | —OC(O)CH₃ | F | H | H |
| 279 | H | —CH₃ | —CH₃ | —OC(O)CH₃ | H | F | H |
| 280 | H | —CH₂CH₃ | —CH₃ | —OC(O)CH₃ | H | F | H |
| 281 | H | —(CH₂)₂CH₃ | —CH₃ | —OC(O)CH₃ | H | F | H |
| 282 | H | —CH(CH₃)₂ | —CH₃ | —OC(O)CH₃ | H | F | H |
| 283 | H | —(CH₂)₃CH₃ | —CH₃ | —OC(O)CH₃ | H | F | H |
| 284 | H | —CH₂—HC═CH₂ | —CH₃ | —OC(O)CH₃ | H | F | H |
| 285 | H | —CH₃ | —CH₃ | H | F | F | H |
| 286 | H | —CH₂CH₃ | —CH₃ | H | F | F | H |
| 287 | H | —(CH₂)₂CH₃ | —CH₃ | H | F | F | H |
| 288 | H | —CH(CH₃)₂ | —CH₃ | H | F | F | H |
| 289 | H | —(CH₂)₃CH₃ | —CH₃ | H | F | F | H |
| 290 | H | —CH₂—HC═CH₂ | —CH₃ | H | F | F | H |
| 291 | H | —CH₃ | —CH₃ | H | H | H | H |
| 292 | H | —CH₂CH₃ | —CH₃ | H | H | H | H |
| 293 | H | —(CH₂)₂CH₃ | —CH₃ | H | H | H | H |
| 294 | H | —CH(CH₃)₂ | —CH₃ | H | H | H | H |
| 295 | H | —(CH₂)₃CH₃ | —CH₃ | H | H | H | H |
| 296 | H | —CH₂—HC═CH₂ | —CH₃ | H | H | H | H |
| 297 | H | —CH₃ | —CH₃ | H | —OCH₃ | H | H |
| 298 | H | —CH₂CH₃ | —CH₃ | H | —OCH₃ | H | H |
| 299 | H | —(CH₂)₂CH₃ | —CH₃ | H | —OCH₃ | H | H |
| 300 | H | —CH(CH₃)₂ | —CH₃ | H | —OCH₃ | H | H |
| 301 | H | —(CH₂)₃CH₃ | —CH₃ | H | —OCH₃ | H | H |
| 302 | H | —CH₂—HC═CH₂ | —CH₃ | H | —OCH₃ | H | H |
| 303 | H | —CH₃ | —CH₃ | H | H | F | H |
| 304 | H | —CH₂CH₃ | —CH₃ | H | H | F | H |
| 305 | H | —(CH₂)₂CH₃ | —CH₃ | H | H | F | H |
| 306 | H | —CH(CH₃)₂ | —CH₃ | H | H | F | H |
| 307 | H | —(CH₂)₃CH₃ | —CH₃ | H | H | F | H |
| 308 | H | —CH₂—HC═CH₂ | —CH₃ | H | H | F | H |
| 309 | H | cyclopropyl | —CH₃ | H | H | H | H |
| 310 | H | cyclopropyl | —CH₃ | H | H | H | H |
| 311 | H | cyclopropyl | —CH₃ | —OH | H | H | H |
| 312 | H | cyclopropyl | —CH₃ | —OC(O)CH₃ | H | H | H |
| 313 | H | cyclopropyl | —CH₃ | H | —OCH₃ | H | H |
| 314 | H | cyclopropyl | —CH₃ | H | H | H | H |
| 315 | H | cyclopropyl | —CH₃ | H | H | H | H |
| 316 | H | cyclopropyl | —CH₃ | —OH | F | H | H |
| 317 | H | cyclopropyl | —CH₃ | —OH | H | H | H |
| 318 | H | cyclopropyl | —CH₃ | —OC(O)CH₃ | H | H | H |
| 319 | H | cyclopropyl | —CH₃ | H | F | H | H |
| 320 | H | cyclopropyl | —CH₃ | H | H | F | H |
| 321 | H | —CD₃ | —CH₃ | H | H | H | H |
| 322 | H | —CD₃ | —CH₃ | —OH | H | H | H |
| 323 | H | —CD₃ | —CH₃ | —OC(O)CH₃ | H | H | H |
| 324 | H | —CD₃ | —CH₃ | H | —OCH₃ | H | H |
| 325 | H | —CD₃ | —CH₃ | H | F | H | H |
| 326 | H | —CD₃ | —CH₃ | —OH | F | H | H |
| 327 | H | —CD₃ | —CH₃ | —OC(O)CH₃ | F | F | H |
| 328 | H | —CD₃ | —CH₃ | H | F | F | H |
| 329 | H | —CD₂CD₃ | H | H | F | H | H |
| 330 | H | —CD₂CD₃ | H | —OH | H | H | H |
| 331 | H | —CD₂CD₃ | H | —OC(O)CH₃ | H | H | H |
| 332 | H | —CD₂CD₃ | H | H | F | F | H |
| 333 | H | —CD₂CD₃ | H | H | H | F | H |
| 334 | H | —CD₂CD₃ | H | —OH | F | H | H |
| 335 | H | —CD₂CD₃ | H | —OC(O)CH₃ | F | H | H |
| 336 | H | —CD₂CD₃ | H | H | —OCH₃ | F | H |

As used herein, the term “5-HT1a” refers to a 5-HT₁a receptor. As used herein, the term “5-HT₂a” refers to a 5-HT₂a receptor. As used herein, “5-HT_{2C}” refers to a 5-HT_{2C} receptor.

As used herein, the term “effective amount” in connection with a compound disclosed herein means an amount capable of treating or preventing a disorder, disease or condition, or symptoms thereof, disclosed herein.

As used herein, the terms “prevent” or “preventing” refers to means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein, the term “treat” or “treating” refers to an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

In further embodiments of the present disclosure, described are novel compounds and compositions, as well as methods of administering the same. In some embodiments, a compound provided herein is for use in the methods provided herein. In some embodiments, the disclosure provides the use of a compound provided herein in the preparation of a medicament for treating one or more of the diseases or disorders provided herein.

As defined herein, a “full agonist” shall mean an agonist having an Emax % of at least 90% for the relevant serotonin receptor agonist assay (e.g., BRET2, calcium mobilization, beta-arrestin) when compared to an industry-accepted control compound for that particular receptor assay (e.g., serotonin (5-OH-tryptamine)). In some embodiments, a “full agonist” will exhibit an Emax % of at least 90%, at least 91%, at least 92%, at least 93%, at least 94%, at least 95%, at least 96%, at least 97%, at least 98%, or at least 99%. Also defined herein, a “partial agonist” shall mean an agonist having an Emax % of less than 90% for the relevant serotonin receptor when compared to an industry-accepted control compound for that particular receptor (e.g., serotonin (5-OH-tryptamine)). In some embodiments, a “partial agonist” will exhibit an Emax % of less than 90%, less than 85%, 80%, 75%, 70%, 65%, 60%, 55%, 50%, 45%, 40%, 35%, 30%, 25%, 20%, 15%, 10%, or even less than 5%. In some embodiments, a partial agonist will exhibit an Emax % of from about 0.1 to about 89.9%, such as about 1 to about 89, about 5 to about 85, about 50 to about 88, about 40 to about 85, about 35 In some embodiments, the compounds of Formula I are selective agonists of 5-HT_{2C}, wherein said compounds are more potent and/or efficacious (Emax) when compared to the other serotonin receptor subtypes (e.g., 5-HT_{2A} and 5-HT_{2B}). Without being bound to any particular theory, in some embodiments, it has been surprisingly discovered that compounds of the present disclosure are orthosteric ligands of 5-HT_{2C} that exhibit a G_{q}-mediated signaling bias and no/minimal β-arrestin recruitment or β-arrestin-mediated intracellular signaling. Applicant theorizes that this G_{q}-biased signaling mechanism results in reduced signal attenuation caused from β-arrestin recruitment, resulting in a greater therapeutic effect and fewer side effects for certain therapeutic indications (e.g., Alzheimer's psychosis, schizophrenia, PWS, addiction, obesity, etc.).

In certain embodiments for compounds of Formula I, Applicant has discovered that the size and nature of alkyl groups for X and/or Y can dramatically affect the metabolism of such compounds. For example, it has been theorized that compounds such as 5-MeO-Dimethyltryptamine (5-MeO-DMT) and Dimethyltryptamine (DMT) are inactive upon oral administration due to rapid metabolism of the methylamino residues by monoamine oxidase (MAO) enzymes. It has also been theorized that the oral stability of psilocin (4-OH-dimethyltryptamine), on the other hand, is due largely to intramolecular coordination (hydrogen bonding) between the 4-OH group and the dimethylamino residue, which effectively shields/inhibits rapid MAO degradation.

Without being bound to any particular scientific theory, Applicant has surprisingly found that, in some embodiments, substituting the alkyl groups X and/or Y with substituents such as deuterium and fluorine can help inhibit MAO degradation of those groups. In addition, or in the alternative, Applicant has discovered that using non-methyl alkyl groups such as ethyl or n-propyl for X and/or Y can also slow or inhibit rapid MAO metabolism upon oral administration. This, in turn, permits the preparation of orally available compounds of Formula I that are highly active serotonergic drugs that do not require special formulating procedures (e.g., dosages containing MAO inhibitors), or the presence of hydrogen bond-forming donors in the molecular structure that—in some cases—can negatively impact the properties of the underlying compound (e.g., reduction of 5-HT₁A and/or 5-HT_{2A} agonism).

The present disclosure relates to compositions comprising, consisting essentially of, or consisting of an effective amount of a compound of Formula I and an excipient. The terms “composition” and “formulation” are used interchangeably herein. Other embodiments relate to pharmaceutical compositions comprising, consisting essentially of, or consisting of a therapeutically effective amount of a compound of Formula I, including those discussed above, and a pharmaceutically acceptable excipient (also known as a pharmaceutically acceptable carrier). As discussed above, a compound of Formula I may be therapeutically useful to prevent and/or treat, for example, PWS, psychological disorders, brain disorders, pain and inflammation as well as other disorders such as those discussed above.

Exemplary pharmaceutically-acceptable carriers include, but are not limited to, any pharmaceutically-acceptable material, composition or vehicle, such as a liquid or solid filler, diluent, excipient, solvent or encapsulating material, which may be involved in carrying or transporting the subject chemical from one organ, or portion of the body, to another organ, or portion of the body, e.g., oral dosage form enabling transport of compound to the stomach and/or intestinal tract. In certain embodiments, such oral dosage forms may comprise a tablet or capsule (e.g., gel or solid capsule). Each carrier is preferably “acceptable” in the sense of being compatible with the other ingredients of the formulation and not injurious to the subject. Some examples of materials which can serve as pharmaceutically-acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In some embodiments, the composition may further comprise one or more pharmaceutically-acceptable antioxidants. In certain embodiments, such antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some embodiments, the compounds described herein may be administered orally to a subject in a liquid dosage form. In some embodiments, such a liquid oral formulation may be desirable, particularly for certain seizure disorders (e.g., DEEs) in which administering solid dosage forms (e.g., tablets) to subjects may be difficult. In some embodiments, the liquid dosage form may comprise the at least one compound, water, and an excipient (e.g., a sugar, such as sucrose). In some embodiments, the liquid dosage form is administered to the subject 2-3 times per day.

In some embodiments, the compounds described herein may be administered orally to a subject in a solid dosage form. In some embodiments, such a solid oral formulation may comprise an extended release tablet, which may be administered once a day. In some embodiments, the extended release tablet may be used to effectively release the at least one compound to the subject over an extended period, such as 8-36 hrs or 12-24 hrs. In some embodiments, the extended release tablet can deliver at least some of the compound to the subject's small intestine.

Within the context of this disclosure, unless otherwise specified, the serotonergic compounds described herein may be present in their protonated or deprotonated (salt or free base) forms or mixtures thereof depending on the context, for example, the pH of the solution or composition. However, in certain embodiments, the serotonergic compounds described herein are lipophilic, meaning they will tend to combine with lipids and fats and can readily pass though biological membranes in the body of an animal or human (e.g., blood brain barrier). In certain embodiments, the serotonergic compound in free base form is lipophilic.

As used herein, the term “salt” refers to a neutralized ionic compound. In one embodiment, a salt is formed from the neutralization of acids and bases. In one embodiment, a salt is electrically neutral.

In one embodiment, the compositions disclosed herein comprise a stabilizer. As used herein, the term “stabilizer” refers to a compound useful for preventing the degradation of an active ingredient, e.g., a compound of Formula I, a psilocybin derivative, a cannabinoid, a terpene, etc. In one embodiment, a stabilizer prevents an active ingredient from degrading. In one embodiment, a stabilizer prevents a serotonergic drug from reacting with other compounds in the composition, e.g., a cannabinoid, a terpene, a base, an acid, etc. In one embodiment, a stabilizer prevents a serotonergic drug from reacting with the ambient atmosphere, e.g., heat, light, water, and/or oxygen. In one embodiment, a stabilizer comprises an antioxidant. In one embodiment, a stabilizer comprises a pH buffer.

In one embodiment, the methods and compositions disclosed herein comprise an antioxidant. As used herein, the term “antioxidant” refers to a compound and/or a composition useful for preventing oxidation. In one embodiment, an antioxidant protects an active ingredient from “free radicals”. Within the context of this disclosure, a “free radical” is an atom, molecule, or an ion with an unpaired valence electron. In one embodiment, an antioxidant is an electron donor.

In one embodiment, an antioxidant is chosen from ascorbic acid, lycopene, tocopherol, melatonin, retinol, astaxanthin, lutein, apigenin, carnosine, selenium, zinc, cucurmin, and a salt or derivative thereof.

In one embodiment, an antioxidant is ascorbic acid and/or its salts or derivatives. Within the context of this disclosure, the term “ascorbic acid” comprises Vitamin C and/or a salt or derivative thereof.

In one embodiment, the methods and compositions disclosed herein comprise a pH buffer.

As used herein, the term “pH buffer” refers to a compound or a composition useful for maintaining the pH of a composition. In one embodiment, a pH buffer comprises a weak acid and a corresponding conjugate base. In one embodiment, a pH buffer comprises a weak base and a corresponding conjugate acid. In one embodiment, a pH buffer does not change the pH of a composition with the addition of a strong acid and/or base.

In one embodiment, a pH buffer maintains the pH of a composition around 7. In one embodiment, a pH buffer maintains the pH of a composition below about 7. In one embodiment, a pH buffer maintains the pH of a composition above about 7. In one embodiment, a pH buffer maintains the pH of a composition ranging from about 2 to about 6. In one embodiment, a pH buffer maintains the pH of a composition ranging from about 5 to about 7. In one embodiment, a pH buffer maintains the pH of a composition ranging from about 6 to about 8. In one embodiment, a pH buffer maintains the pH of a composition ranging from about 7 to about 10.

In one embodiment, a pH buffer comprises citric acid, acetic acid, monosodium phosphate, N-Cyclohexyl-2-aminoethanesulfonic acid, borate, hydrochloric acid, and/or sodium hydroxide.

In one embodiment, the methods disclosed herein comprise administering a composition comprising an acid.

As used herein, the term “acid” refers to a molecule or ion capable of donating a proton, i.e., H⁺ and/or accepting electrons. In one embodiment, an “acid” refers to a Lewis acid. In one embodiment, an “acid” refers to a Bronsted acid. In one embodiment, an acid is determined by a composition's pH. In one embodiment, a pH below 7 indicates the presence of an acid.

In one embodiment, the compositions and methods disclosed herein comprise administering a formulation comprising a base.

As used herein, the term “base” refers to a molecule or ion capable of accepting a proton, i.e., an H⁺. In one embodiment, a “base” refers to a molecule capable of donating an electron pair, i.e., a Lewis base. In one embodiment, the presence of a base is determined by a compound's pH. In one embodiment, a pH above 7 indicates the presence of a base.

In one embodiment, the compositions and methods disclosed herein comprise administering a non-water soluble composition.

In some embodiments, the compositions described herein are non-aqueous.

As used herein, the term “water soluble” refers to a compound or composition capable of dissolving in water at standard temperature and pressure. In one example, 1 g of a compound dissolves in 1 L of water. In one example, 2 g of a compound dissolves in 1 L of water. In one example, 5 g of a compound dissolves in 1 L of water. In one example, 10 g of a compound dissolves in 1 L of water. In one embodiment, a compound's solubility in water is an inherent property of a compound. In one embodiment, a compound's solubility in water is facilitated by another compound, e.g., an excipient.

In one embodiment, the compositions and methods disclosed herein comprise administering a compound of Formula I present as and/or within a homogenous mixture within a dosage formulation.

As used herein, the term “administering” (e.g., administering a drug) refers to dosing, treating, giving, and/or providing. In one embodiment, administering a compound of Formula I comprises providing the compound to an organism (e.g., a human being). In one embodiment, administering a compound of Formula I and a glucagon-like peptide-1 (GLP-1) receptor agonist comprises providing both compounds to an organism (e.g., a human being) simultaneously, sequentially, or separately.

In one embodiment, administering a neurotransmitter activity modulator comprises providing a neurotransmitter activity modulator to an organism (e.g., a human being) with a neurotransmitter receptor. In one embodiment, administering a neurotransmitter activity modulator comprises providing a neurotransmitter activity modulator along with a compound of Formula I, e.g., a formulation having each of a neurotransmitter activity modulator and a compound of Formula I in a single dosage. In one embodiment, administering a neurotransmitter activity modulator comprises applying a transdermal composition, e.g., applying a topical composition to the skin having each of a neurotransmitter activity modulator and a compound of Formula I. In one embodiment, administering a neurotransmitter activity modulator comprises giving a transmucosal preparation, e.g., providing rapidly dissolving a tablet with an absorption enhancer having each of a neurotransmitter activity modulator and a compound of Formula I.

As used herein, the term “identifying a person in need of treatment” refers to analyzing, diagnosing, and/or determining whether a person requires treatment for a disease or condition. In one embodiment, identifying a person in need of treatment comprises diagnosing a person with a medical condition, e.g., a neurological disorder, a chemical imbalance, a hereditary condition, etc. In one embodiment, identifying a person in need of treatment comprises performing a psychiatric evaluation. In one embodiment, identifying a person in need of treatment comprises performing a blood test. In one embodiment, identifying a person in need of treatment comprises determining whether a person has a compulsive disorder. In one embodiment, identifying a person in need of treatment comprises self-identifying as having a compulsive disorder.

In some embodiments, the compositions described herein comprise a serotonergic drug, wherein the serotonergic drug is selected from Formula I. In some embodiments, the composition comprises a single serotonergic drug. In some embodiments, the serotonergic drug consists essentially of a compound of Formula I.

In some embodiments, a compound of Formula I may be administered to a subject, wherein the administration is guided by certain contraindications. As described herein, a “contraindicated” compound for a method of treatment described herein is secondary compound that should not be used in a particular patient/subject due to a high risk of harm, serious adverse effects, or potential life-threatening interactions. For example, in certain embodiments, adverse drug-drug interactions could take place when a subject receiving a compound of Formula I is also administered a monoamine oxidase inhibitor that reduces or inhibits the in vivo metabolism of the compound of Formula I. Therefore, in certain embodiments, the compounds of Formula I should only be administered to subjects that are not also receiving contraindicated secondary compounds, such as a serotonin receptor agonist, a serotonin receptor antagonist, an SNRI, an SSRI, or a monoamine oxidase inhibitor. Exemplary serotonin receptor agonists include, but are not limited to, fenfluramine, lorcaserin, and bexicaserin.

Although the disclosure has been described with reference to various exemplary embodiments, it is to be understood that these embodiments are merely illustrative of the principles and applications of the present disclosure. Those having skill in the art would recognize that various modifications to the exemplary embodiments may be made, without departing from the scope of the disclosure.

Where reference is made to a particular compound, it should be understood that this disclosure also contemplates salts and derivatives of that compound as well as degradation products, such as oxidized versions of explicitly disclosed molecules.

Moreover, it should be understood that various features and/or characteristics of differing embodiments herein may be combined with one another. It is, therefore, to be understood that numerous modifications may be made to the illustrative embodiments and that other arrangements may be devised without departing from the scope of the disclosure.

Furthermore, other embodiments of the disclosure will be apparent to those skilled in the art from consideration of the specification and practice of the disclosure disclosed herein. It is intended that the specification and examples be considered as exemplary only, with a scope and spirit being indicated by the claims.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5%, or +1% of the indicated range, value, or structure, unless otherwise indicated.

### EXAMPLES

### Example 1

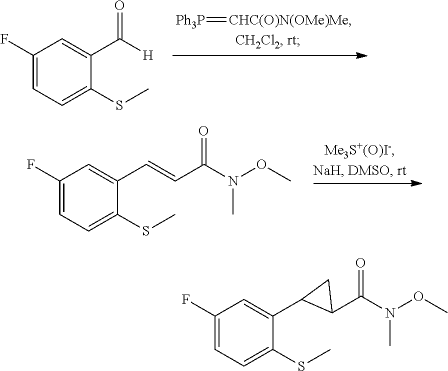

To a solution of 5-fluoro-2-(methylthio)benzaldehyde (1.0 mmol) in anhydrous dichloromethane (10 mL) was added N-methoxy-N-methyl(triphenylphosphoranylidene)acetamide (1.5 mmol), and the solution was stirred at room temperature overnight. The solution was then concentrated, and the residue was purified by silica gel chromatography using 15% ethylacetate in hexanes to afford (E)-3-(5-fluoro-2-(methylthio)phenyl)-N-methoxy-N-methylacrylamide.

Trimethylsulfoxonium iodide (1.0 mmol) was suspended in anhydrous DMSO (5 mL), and sodium hydride (1.0 mmol) was added in small portions. The mixture was stirred at room temperature for 1 hour to afford a clear solution. A solution of (E)-3-(5-fluoro-2-(methylthio)phenyl)-N-methoxy-N-methylacrylamide (0.6 mmol) in anhydrous DMSO (5 mmol) was then slowly added and the solution was stirred at room temperature overnight. Workup with water and EtOAc followed by purification by silica gel chromatography using 15% ethylacetate in hexanes afforded 2-(5-fluoro-2-(methylthio)phenyl)-N-methoxy-N-methylcyclopropane-1-carboxamide.

### Example 2

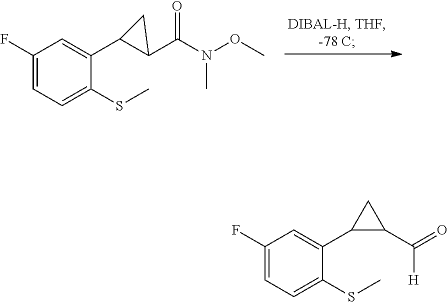 [SMILES: CON(C)C(=O)C1CC1c1cc(F)ccc1SC ; CSc1ccc(F)cc1C1CC1C=O]

A solution of 2-(5-fluoro-2-(methylthio)phenyl)-N-methoxy-N-methylcyclopropane-1-carboxamide (0.5 mmol) in anhydrous THF (10 mL) was cooled to −78 °C under argon. To this solution was added slowly DIBAL-H (1.0 M solution in THF, 1.0 mmol) and the solution was stirred at −78 °C for 3 hours. Saturated aqueous solution of Rochelle's salt was added to quench the reaction and the mixture was warmed to room temperature, stirred for 1 hour and filtered. The solid was washed with EtOAc and the filtrate is extracted with EtOAc. The combined organic phases were washed with brine, dried over sodium sulfate, and concentrated to afford 2-(5-fluoro-2-(methylthio)phenyl)cyclopropane-1-carbaldehyde.

### Example 3

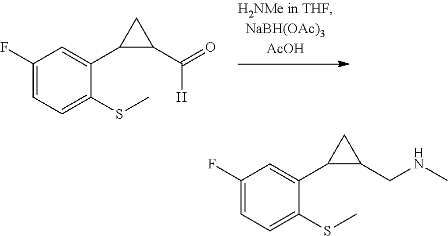

To a suspension of methyl amine (0.1 mL of 2.0M in THF) and AcOH (0.1 mL) and THF (10 mL) was added sodium triacetoxy borohydride (0.3 mmol) followed by the slow addition of 2-(5-fluoro-2-(methylthio)phenyl)cyclopropane-1-carbaldehyde (0.1 mmol) in THF (5 mL). The reaction was stirred under argon for 3 hours at room temperature before quenching with water and extracting with DCOM. The organic phases were washed with brine, dried over sodium sulfate, concentrated, and purified by silica gel chromatography using 7% MeOH in DCM to afford the free base 1-(2-(5-fluoro-2-(methylthio)phenyl)cyclopropyl)-N-methylmethanamine.

### Example 4

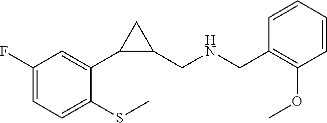 [SMILES: COc1ccccc1CNCC1CC1c1cc(F)ccc1SC]

### Example 3 is repeated using 2-methoxybenzylamine as the primary amine to afford 1-(2-(5-fluoro-2-(methylthio)phenyl)cyclopropyl)-N-(2-methoxybenzyl)methanamine

### Example 5

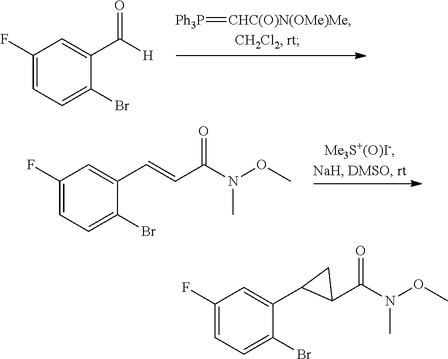

### Example 1 is repeated using 2-bromo-5-fluorobenzaldehyde as the starting material to afford 2-(2-bromo-5-fluorophenyl)-N-methoxy-N-methylcyclopropane-1-carboxamide

### Example 6

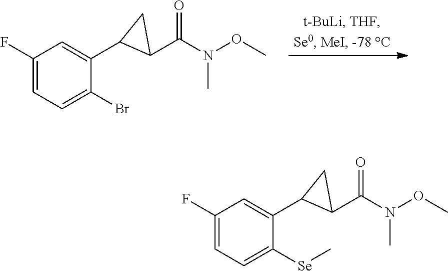 [SMILES: CON(C)C(=O)C1CC1c1cc(F)ccc1Br ; CON(C)C(=O)C1CC1c1cc(F)ccc1[Se]C]

Under an inert atmosphere was added tert butyl lithium (1.25 mL of 1.6M in hexanes) to a stirred solution of 2-(2-bromo-5-fluorophenyl)-N-methoxy-N-methylcyclopropane-1-carboxamide (1 mmol) in anhydrous THF (40 mL) at −78 C. After 30 minutes the reaction was allowed to warm to room temperature and selenium (1 mmol, <5 mm) was added under a brisk stream of nitrogen and the reaction was further purged with nitrogen for 5 minutes. A solution of methyl iodide (1.2 mL of 1.0M in THF) was added slowly to the reaction mixture and 20 minutes the reaction was concentrated in vacuo, then redissolved in dichloromethane and washed with water. The organic phase was separated, dried over sodium sulfate, filtered, and concentrated before purification via silica gel chromatography with 4% MeOH in DCM to afford 2-(5-fluoro-2-(methylselanyl)phenyl)-N-methoxy-N-methylcyclopropane-1-carboxamide.

### Example 7

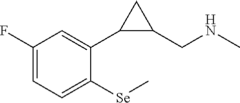

The 2-(5-fluoro-2-(methylselanyl)phenyl)-N-methoxy-N-methylcyclopropane-1-carboxamide product of Example 6 is subjected to the reaction conditions of Examples 2 and 3 to afford the 1-(2-(5-fluoro-2-(methylselanyl)phenyl)cyclopropyl)-N-methylmethanamine product.

### Example 8

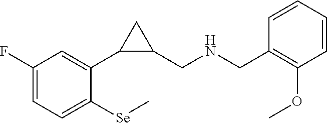 [SMILES: COc1ccccc1CNCC1CC1c1cc(F)ccc1[Se]C]

The 2-(5-fluoro-2-(methylselanyl)phenyl)-N-methoxy-N-methylcyclopropane-1-carboxamide product of Example 6 is subjected to the reaction conditions of Examples 2 and 4 to afford 1-(2-(5-fluoro-2-(methylselanyl)phenyl)cyclopropyl)-N-methylmethanamine.

### Example 9

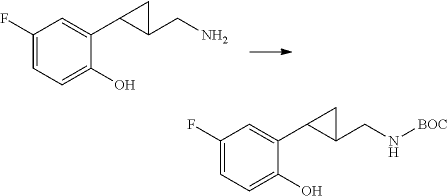 [SMILES: NCC1CC1c1cc(F)ccc1O ; CC(C)(C)OC(=O)NCC1CC1c1cc(F)ccc1O]

The racemic starting material, trans-2-(5-fluorophenyl-2-hydroxy)cyclopropylmethylamine, is prepared according to the procedure described in Cho et al., J. Med. Chem. 2009, 52, 1885, which is incorporated herein by reference. To a solution of trans-2-(5-fluorophenyl-2-hydroxy)cyclopropylmethylamine (55.2 mmol) and Boc₂O (60.7 mmol) in CH₂Cl₂ is added triethylamine (221 mmol) at 0 °C. The mixture is stirred at 0 °C for 30 min and then at rt for 30 min. To the resulting mixture is added sat. aq NaHCO₃. The organic layer is further washed with water (A-1), dried over Na₂SO₄, and concentrated in vacuo. The residue is purified by flash chromatography using gradient elution from 0% EtOAc-hexane to 30% EtOAc-hexane to afford the protected carbamate as a white solid.

### Example 10

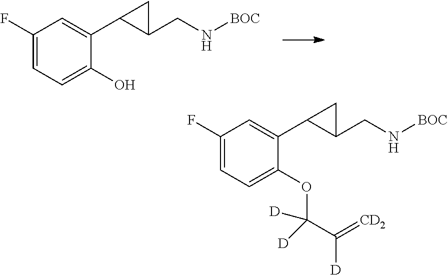

To a solution of the BOC-protected amine (17.8 mmol) in DMF are added K₂CO₃ (71.1 mmol) and allyl-d5 bromide (71.1 mmol). After the mixture is stirred at 60 °C in a microwave reactor for 2 hrs, the organic layer is washed with brine, dried over Na₂SO₄, filtered, and concentrated. The crude product is purified by flash chromatography using gradient elution from 0% EtOAc-hexane to 20% EtOAc-hexane to afford the racemic target compound.

### Example 11

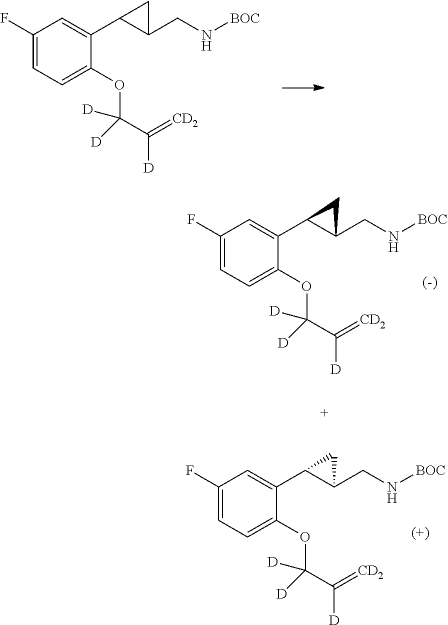

The BOC-protected racemate prepared according to the method of Example 10 is separated by chiral HPLC (Hexane/iPrOH, 96/4 isocratic, stack injections, flowrate of 12 mL/min, λ=208 nm) using chiralpak AD column to afford the (+)-isomer and the (−)-isomer.

### Example 12

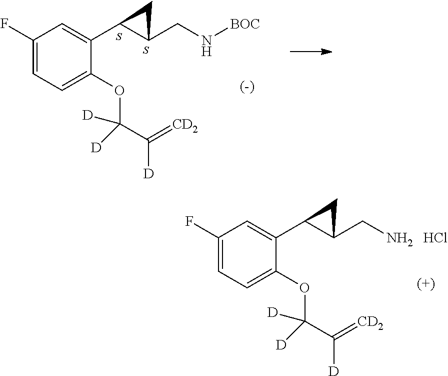

The (−) isomer produced according to Example 11 (17.3 mmol) is dissolved in a 2 N HCl solution in diethyl ether (86 mmol), and the reaction mixture is stirred at ambient temperature for 48 h. A white precipitate will form after several hours, and the mixture is stirred until the reaction was complete by TLC. The crude precipitate is filtered and purified by recrystallization from ethanol/Et₂O to afford the target compound as a white powder.

### Example 13

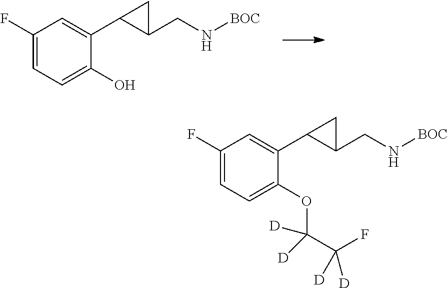 [SMILES: CC(C)(C)OC(=O)NCC1CC1c1cc(F)ccc1O ; [2H]C([2H])(F)C([2H])([2H])Oc1ccc(F)cc1C1CC1CNC(=O)OC(C)(C)C]

The d4 BOC racemate product is produced according to the procedure set forth in PCT Patent Publication WO2020/260196 (see, e.g., pages 30 and 31), which is incorporated herein by reference in its entirety, except phenol 1 is replaced with the BOC-protected compound prepared according to Example 9 above.

### Example 14

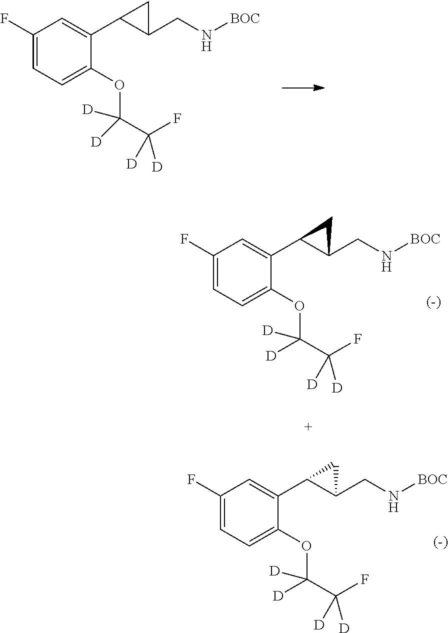 [SMILES: [2H]C([2H])(F)C([2H])([2H])Oc1ccc(F)cc1C1CC1CNC(=O)OC(C)(C)C ; [2H]C([2H])(F)C([2H])([2H])Oc1ccc(F)cc1[C@H]1C[C@@H]1CNC(=O)OC(C)(C)C ; [2H]C([2H])(F)C([2H])([2H])Oc1ccc(F)cc1[C@@H]1C[C@H]1CNC(=O)OC(C)(C)C]

The d4 BOC-protected racemate prepared according to the method of Example 13 is separated by chiral HPLC (Hexane/iPrOH, 96/4 isocratic, stack injections, flowrate of 12 mL/min, λ=280 nm) using chiralpak AD column to afford the (+)-isomer and the (−)-isomer.

### Example 15

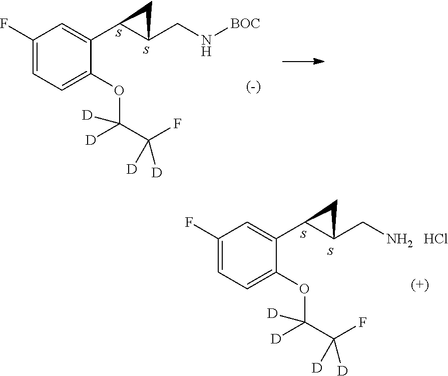

The (−) isomer produced according to Example 14 (17.3 mmol) is dissolved in a 2 N HCl solution in diethyl ether (86 mmol), and the reaction mixture is stirred at ambient temperature for 48 h. A white precipitate will form after several hours, and the mixture is stirred until the reaction was complete by TLC. The crude precipitate is filtered and purified by recrystallization from ethanol/Et₂O to afford the target compound as a white powder.

### Example 16

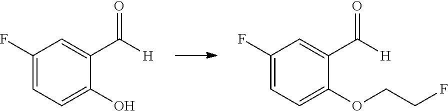 [SMILES: O=Cc1cc(F)ccc1O ; O=Cc1cc(F)ccc1OCCF]

To a solution of the starting phenol (2.8 g, 20 mmol) and 2-iodo-1-fluoroethane (2 equiv) in anhydrous DMF (1 M) was added Cs₂CO₃ (2 equiv) at room temperature. The reaction mixture was heated at 50 °C until the reaction was complete as indicated by TLC analysis. The reaction mixture was allowed to cool to room temperature and diluted with water and diethyl ether. The phases were separated and the aqueous phase was extracted with diethyl ether (×3). The combined organic extracts were washed with water (×2) and brine, and dried over anhydrous sodium sulfate. After filtration, the solvent was removed in vacuo to provide a crude solid that was used in the next step without purification.

### Example 17

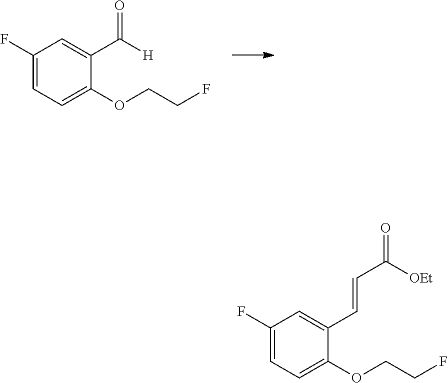 [SMILES: O=Cc1cc(F)ccc1OCCF ; CCOC(=O)/C=C/c1cc(F)ccc1OCCF]

To a vigorously stirred suspension of NaH (60% mineral oil, 1.4 equiv) in THF (50 mL) was slowly added triethyl phosphonoacetate (1.2 equiv) dropwise at room temperature. Following the cessation of gas evolution, a 1 M THF solution of crude product of Example 16 was added dropwise at room temperature. The reaction was quenched after 30 min by the careful addition of water and diethyl ether. The phases were separated and the aqueous phase was extracted with diethyl ether (×3). The combined organic extracts were combined and washed with water (×2) and brine, and dried with anhydrous sodium sulfate. After filtration, the solvent was removed in vacuo to provide the crude solid product that was used in the next step without purification.

### Example 18

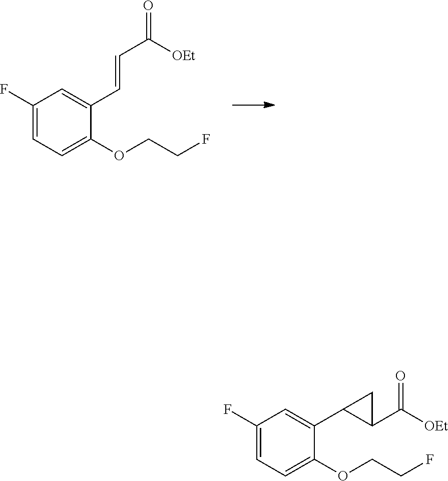 [SMILES: CCOC(=O)/C=C/c1cc(F)ccc1OCCF ; CCOC(=O)C1CC1c1cc(F)ccc1OCCF]

To a vigorously stirred suspension of trimethylsulfoxonium iodide (1.5 equiv) in DMSO (0.2 M) was added portion wise NaH (60% mineral oil, 1.5 equiv) at room temperature. The reaction mixture was allowed to stir until the reaction mixture became homogeneous (ca. 45-60 min) at which point a 1 M DMSO solution of the crude product produced in Example 17 was added dropwise. The reaction mixture was allowed to stir for at least 2 hours (or sooner if TLC indicated a complete reaction) and quenched by the addition of water and diethyl ether. The phases were separated, and the aqueous phase was extracted with diethyl ether (×3). The combined organic extracts were combined and washed with water (×2) and brine, and dried over anhydrous sodium sulfate. After filtration, the solvent was removed in vacuo to provide a crude oil that was purified by a flash silica gel plug (dry load, hexane then 15% EA/Hex) to provide 1.26 g of the desired ester product as colorless oil (36% yield over 3 steps).

### Example 19

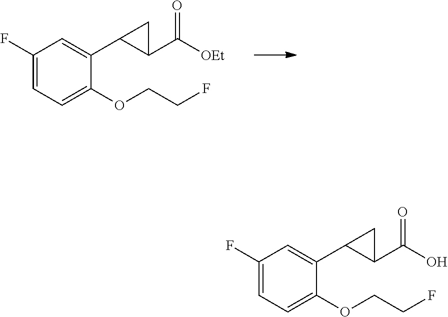 [SMILES: CCOC(=O)C1CC1c1cc(F)ccc1OCCF ; O=C(O)C1CC1c1cc(F)ccc1OCCF]

To a stirred solution of the ester product of Example 18 in THF (20 mL) was added an aqueous solution of lithium hydroxide monohydrate (3.0 equiv) in water (20 mL). The reaction mixture was heated to 60 °C and stirred overnight. The reaction mixture was then allowed to cool to room temperature and diluted with diethyl ether. The phases were separated and the organics washed with sat. aq. sodium bicarbonate (×2). The combined aqueous extracts were acidified to pH=1 and extracted with dichloromethane (×3). The combined organic extracts were dried over anhydrous sodium sulfate, filtered, and concentrated in vacuo to afford the acid product as a white solid.

### Example 20

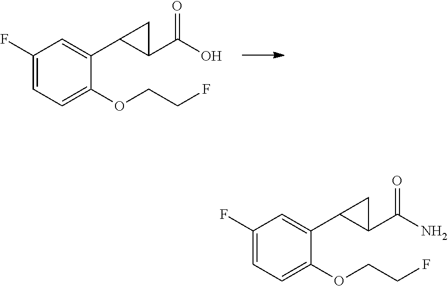 [SMILES: O=C(O)C1CC1c1cc(F)ccc1OCCF ; NC(=O)C1CC1c1cc(F)ccc1OCCF]

To a stirred solution of 365 mg of the acid product from Example 19 in DMF (0.1 M) at room temperature was added 1,1′-carbonyldiimidazole (2.0 equiv). The reaction mixture was allowed to stir at room temperature for 30 min before ammonium acetate (10 equiv) and triethyl amine (5 equiv) are added. The reaction mixture is allowed to stir at room temperature overnight before being quenched by the addition of diethyl ether and 1M HCl. The phases were separated and the aqueous was extracted with diethyl ether (×3). The combined organic extracts were washed with 1M HCl, sat. aq. sodium bicarbonate, water, and brine, and dried over anhydrous sodium sulfate. After filtration, the solvent was removed in vacuo to afford the amide product which was used in the next step without additional purification.

### Example 21

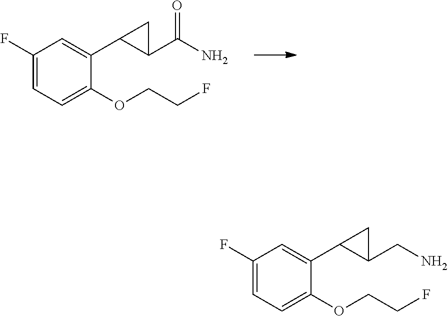 [SMILES: NC(=O)C1CC1c1cc(F)ccc1OCCF ; NCC1CC1c1cc(F)ccc1OCCF]

To a stirred solution of the amide produced in Example 20 in THF (0.1 M) was added dropwise BH₃-THF (1 M solution in THF, 5 equiv). The reaction mixture was refluxed for 4 hours before cooling to room temperature and quenching with methanol. The reaction mixture was concentrated to dryness, diluted with methanol (0.1 M) and 6 M HCl (3 mL), and refluxed for 30 min. After cooling to room temperature, the methanol was removed in vacuo and the crude mixture was basified with 3 M NaOH aqueous solution and extracted with DCM (×5). The combined organic extracts were washed with brine and dried with anhydrous sodium sulfate, filtered, and concentrated in vacuo to afford the target as solid. Formation of the free base target compound was confirmed by HRMS (M+H)+(Found: 228.1204 m/z; Calc'd: 228.1194 m/z) and ¹H NMR (600 MHz, d₄-MeOH) δ 6.94 (dd, J=9.0, 4.6 Hz, 1H), 6.89 (ddd, J=9.0, 8.0, 3.0 Hz, 1H), 6.76 (dd, J=9.4, 3.0 Hz, 1H), 4.92-4.70 (m, 2H), 4.38-4.16 (m, 2H), 3.02 (d, J=7.5 Hz, 2H), 3.05-2.99 (m, 2H), 2.16 (dt, J=9.0, 5.3 Hz, 1H), 1.31-1.22 (m, 1H), 1.18 (dt, J=8.5, 5.5 Hz, 1H), 1.04 (dt, J=9.0, 5.2 Hz, 1H).

### Example 22

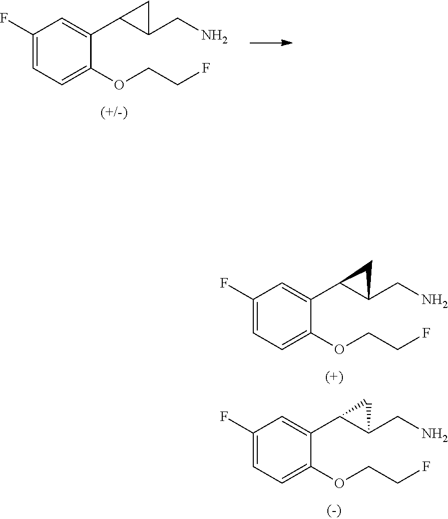 [SMILES: NCC1CC1c1cc(F)ccc1OCCF ; NC[C@H]1C[C@@H]1c1cc(F)ccc1OCCF ; NC[C@@H]1C[C@H]1c1cc(F)ccc1OCCF]

The racemate free base product prepared according to the method of Example 21 was separated by chiral HPLC, hexane/iPrOH/DEA, 90/10 (0.1% diethylamine), 5 μl injection, flowrate of 10 mL/min, λ=280 nm, using Chiralpak AD-H column to afford the (+)-isomer (20 min) and the (−)-isomer (25 min).

### Example 23

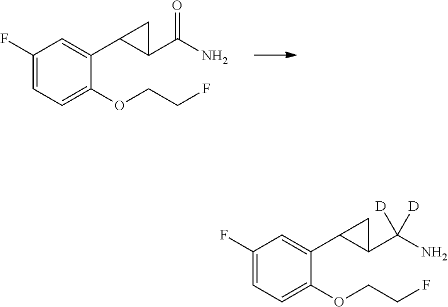 [SMILES: NC(=O)C1CC1c1cc(F)ccc1OCCF ; [2H]C([2H])(N)C1CC1c1cc(F)ccc1OCCF]

To a stirred solution of the amide produced in Example 20 in THF (0.1 M) in an ice bath was added dropwise lithium aluminum deuteride (10 equiv) portion wise. The reaction mixture was allowed to warm to room temperature and additional lithium aluminum deuteride (5 equiv) was added. The reaction mixture was quenched at 0 °C by the addition of water and basified with 3 M KOH solution. The aqueous phase was extracted with DCM (×5) and dried over anhydrous sodium sulfate, filtered, and concentrated in vacuo to afford the alpha-d2 free base racemate of the desired product as a white solid. Formation of the target d2 free base was confirmed by HRMS (ESI+) calc'd for C₁₂H₁₄D₂F₂NO⁺ [M+H]⁺: 230.1320; found: 230.1310; and ¹H NMR (600 MHz, d₄-MeOH) δ 6.94 (dd, J=9.0, 4.6 Hz, 1H), 6.89 (ddd, J=9.0, 8.0, 3.0 Hz, 1H), 6.76 (dd, J=9.4, 3.0 Hz, 1H), 4.95-4.69 (m, 2H), 4.35-4.17 (m, 2H), 2.16 (dt, J=9.0, 5.3 Hz, 1H), 1.25 (dt, J=9.7, 4.9 Hz, 1H), 1.18 (dt, J=8.5, 5.5 Hz, 1H), 1.04 (dt, J=9.0, 5.2 Hz, 1H).

### Example 24

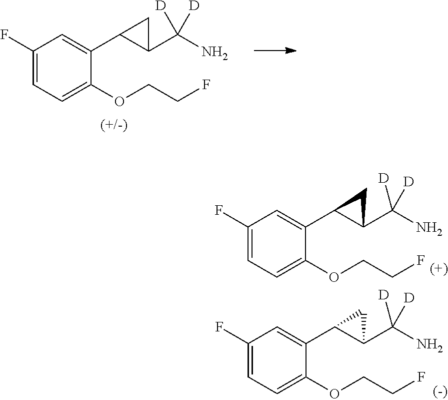

The racemate free base product prepared according to the method of Example 23 was separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer (20 min) and the (−)-isomer (25 min).

### Example 25

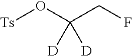 [SMILES: [2H]C([2H])(CF)OS(=O)(=O)c1ccc(C)cc1]

To a solution of ethyl fluoroacetate (10 mmol) in diethyl ether (50 mL) was added an excess of lithium aluminum deuteride (3.0 equiv) portion wise at 0 °C. Upon completion of the reaction as indicated by TLC, the reaction mixture was quenched carefully with water and acidified with 1 M HCl. The phases were separated, and the aqueous phase was extracted with DCM (×3). The combined organic extracts were dried over anhydrous sodium sulfate and filtered. To the filtrate was added tosyl chloride (1.7 equiv), triethylamine (2.0 equiv) and 5 mol % DMAP. The reaction mixture was allowed to stir overnight before being concentrated and dry loaded onto a silica gel column (elution: 5, 10, 15, 25% ethyl acetate in hexane) to provide 2-fluoroethyl-1,1-d2 4-methylbenzenesulfonate as a colorless oil (73% yield).

### Example 26

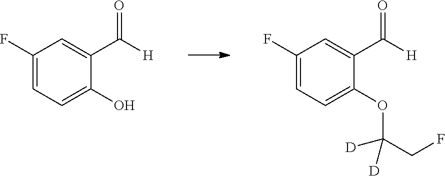 [SMILES: O=Cc1cc(F)ccc1O ; [2H]C([2H])(CF)Oc1ccc(F)cc1C=O]

The procedure of Example 16 was repeated, except 2-iodo-1-fluoroethane was replaced with TsOCD₂CH₂F prepared according to Example 25 to afford the free base of the target d2 product.

### Example 27

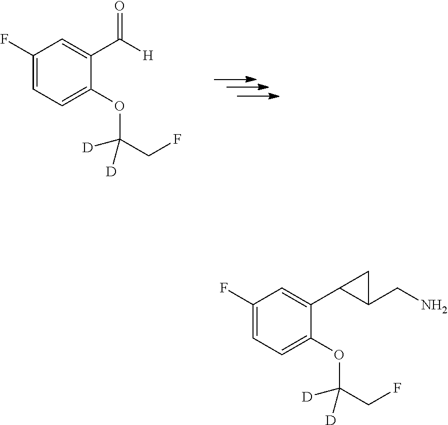 [SMILES: [2H]C([2H])(CF)Oc1ccc(F)cc1C=O ; [2H]C([2H])(CF)Oc1ccc(F)cc1C1CC1CN]

The procedure of Example 17 was repeated, except the aldehyde was replaced with the d2 aldehyde produced according to Example 26. The procedures of Examples 18 through 21 were then followed with the d2 intermediates to afford the free base of the target d2 racemate. Formation of the desired product was confirmed by HRMS (ESI+) calc'd for C₁₂H₁₄D₂F₂NO⁺[M+H]⁺: 230.1320; found 230.1314; and ¹H NMR (600 MHz, d₄-MeOH) δ 6.94 (dd, J=9.0, 4.6 Hz, 1H), 6.89 (ddd, J=9.0, 8.0, 3.1 Hz, 1H), 6.76 (dd, J=9.4, 3.0 Hz, 1H), 4.90-4.72 (m, 2H), 3.07-2.98 (m, 2H), 2.16 (dt, J=9.0, 5.3 Hz, 1H), 1.30-1.21 (m, 1H), 1.18 (dt, J=8.5, 5.5 Hz, 1H), 1.04 (dt, J=9.0, 5.2 Hz, 1H).

### Example 28

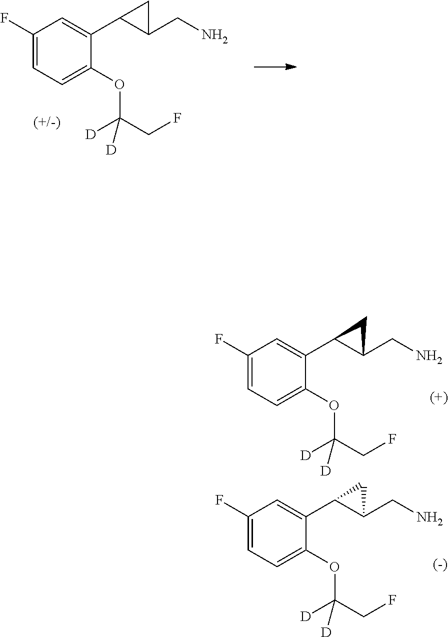

The racemate free base product prepared according to the method of Example 27 was separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer (20 min) and the (−)-isomer (25 min).

### Example 29

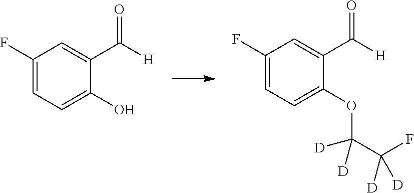 [SMILES: O=Cc1cc(F)ccc1O ; [2H]C([2H])(F)C([2H])([2H])Oc1ccc(F)cc1C=O]

The procedure of Example 16 was repeated, except 2-iodo-1-fluoroethane was replaced with TsOCD₂CD₂F prepared according to the method set forth in WO2020/260196A1 (incorporated herein by reference in its entirety), to afford the free base of the target d4 aldehyde product.

### Example 30

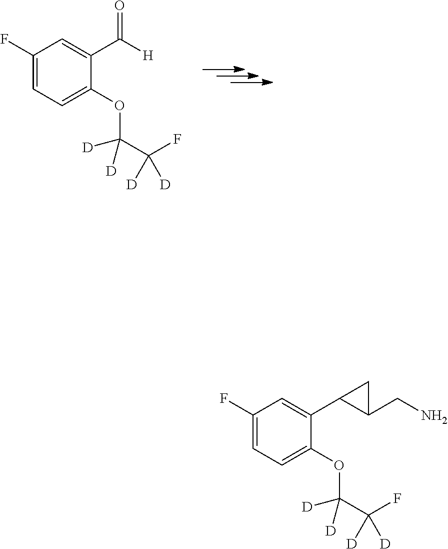 [SMILES: [2H]C([2H])(F)C([2H])([2H])Oc1ccc(F)cc1C=O ; [2H]C([2H])(F)C([2H])([2H])Oc1ccc(F)cc1C1CC1CN]

The procedure of Example 17 was repeated, except the aldehyde was replaced with the d4 aldehyde produced according to Example 29. The procedures of Examples 18 through 21 were then followed with the d4 intermediates to afford the free base of the target d4 racemate. Formation of the desired product was confirmed by HRMS (ESI+) calc'd for C₁₂H₁₂D₄F₂NO⁺ [M+H]⁺: 232.1446; found 232.1440; and ¹H NMR (600 MHz, d₄-MeOH) δ 6.94 (dd, J=9.0, 4.6 Hz, 1H), 6.89 (ddd, J=9.0, 8.0, 3.1 Hz, 1H), 6.76 (dd, J=9.4, 3.1 Hz, 1H), 3.09-2.97 (m, 2H), 2.16 (dt, J=9.0, 5.3 Hz, 1H), 1.30-1.23 (m, 1H), 1.18 (dt, J=8.5, 5.5 Hz, 1H)1.04 (dt, J=9.0, 5.2 Hz, 1H).

### Example 31

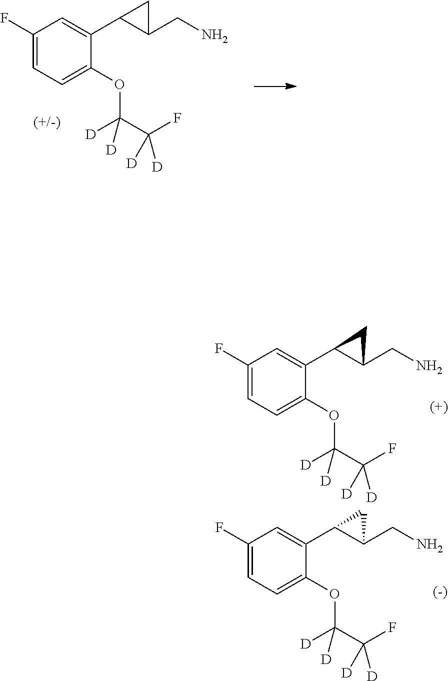

The racemate free base product prepared according to the method of Example 31 was separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer (20 min) and the (−)-isomer (25 min).

### Example 32 Production of Hydrofumarate Salts (Aka [1:1] Fumarate Salts)

[1:1]hydrofumarate salts of each one of the compounds are separately produced from the compounds of Examples 3, 4, 7, and 8 using the following procedure:

1 equiv of the free base product is dissolved in acetone and is added dropwise to a boiling solution of fumaric acid (1 equiv) in acetone. A precipitate forms immediately and the precipitate/acetone is stored overnight at −20 °C. The solids are then filtered and washed with ice-cold acetone to yield the desired hydrofumarate salt.

### Example 33 Production of Fumarate Salts (Aka [2:1] Fumarate Salts)

[2:1] fumarate salts of each one of the compounds are separately produced from the compounds of Examples 3, 4, 7, and 8 using the following procedure:

1 equiv of the free base product is dissolved in acetone and is added dropwise to a boiling solution of fumaric acid (0.5 equiv) in acetone. A precipitate forms immediately and the precipitate/acetone is stored overnight at −20 °C. The solids are then filtered and washed with ice-cold acetone to yield the desired fumarate salt.

### Example 34

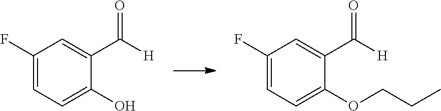

To a solution of the starting phenol (2.8 g, 20 mmol) and 1-iodopropane (2 equiv) in anhydrous DMF (1 M) is added Cs₂CO₃ (2 equiv) at room temperature. The reaction mixture is heated at 50 °C until the reaction was complete as indicated by TLC analysis. The reaction mixture is allowed to cool to room temperature and diluted with water and diethyl ether. The phases are separated and the aqueous phase was extracted with diethyl ether (×3). The combined organic extracts are washed with water (×2) and brine, and dried over anhydrous sodium sulfate. After filtration, the solvent is removed in vacuo to provide a crude solid that is used in the next step without purification.

### Example 35

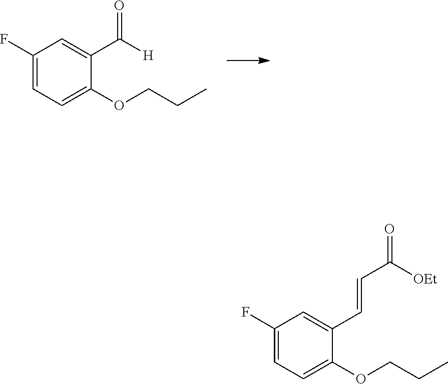 [SMILES: CCCOc1ccc(F)cc1C=O ; CCCOc1ccc(F)cc1/C=C/C(=O)OCC]

To a vigorously stirred suspension of NaH (60% mineral oil, 1.4 equiv) in THF (50 mL) is slowly added triethyl phosphonoacetate (1.2 equiv) dropwise at room temperature. Following the cessation of gas evolution, a 1 M THF solution of crude product of Example 34 is added dropwise at room temperature. The reaction is quenched after 30 min by the careful addition of water and diethyl ether. The phases are separated and the aqueous phase is extracted with diethyl ether (×3). The combined organic extracts are combined and washed with water (×2) and brine, and dried with anhydrous sodium sulfate. After filtration, the solvent is removed in vacuo to provide the crude solid product that was used in the next step without purification.

### Example 36

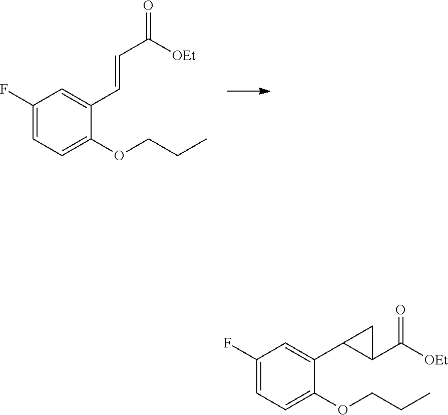 [SMILES: CCCOc1ccc(F)cc1/C=C/C(=O)OCC ; CCCOc1ccc(F)cc1C1CC1C(=O)OCC]

To a vigorously stirred suspension of trimethylsulfoxonium iodide (1.5 equiv) in DMSO (0.2 M) is added portion wise NaH (60% mineral oil, 1.5 equiv) at room temperature. The reaction mixture is allowed to stir until the reaction mixture becomes homogeneous (ca. 45-60 min) at which point a 1 M DMSO solution of the crude product produced in Example 35 is added dropwise. The reaction mixture is allowed to stir for at least 2 hours (or sooner if TLC indicated a complete reaction) and quenched by the addition of water and diethyl ether. The phases are separated, and the aqueous phase is extracted with diethyl ether (×3). The combined organic extracts are combined and washed with water (×2) and brine, and dried over anhydrous sodium sulfate. After filtration, the solvent is removed in vacuo to provide a crude oil that is purified by a flash silica gel plug (dry load, hexane then 15% EA/Hex) to provide the desired ester product as colorless oil.

### Example 37

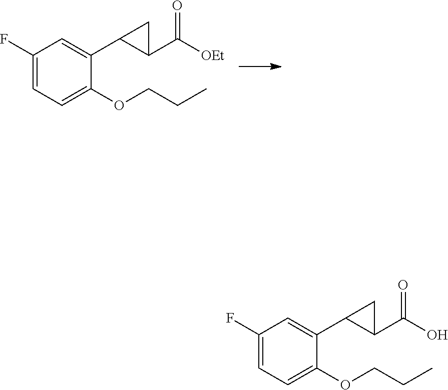 [SMILES: CCCOc1ccc(F)cc1C1CC1C(=O)OCC ; CCCOc1ccc(F)cc1C1CC1C(=O)O]

To a stirred solution of the ester product of Example 36 in THF (20 mL) is added an aqueous solution of lithium hydroxide monohydrate (3.0 equiv) in water (20 mL). The reaction mixture is heated to 60 °C and stirred overnight. The reaction mixture is then allowed to cool to room temperature and diluted with diethyl ether. The phases are separated and the organics washed with sat. aq. sodium bicarbonate (×2). The combined aqueous extracts are acidified to pH=1 and extracted with dichloromethane (×3). The combined organic extracts are dried over anhydrous sodium sulfate, filtered, and concentrated in vacuo to afford the acid product as a white solid.

### Example 38

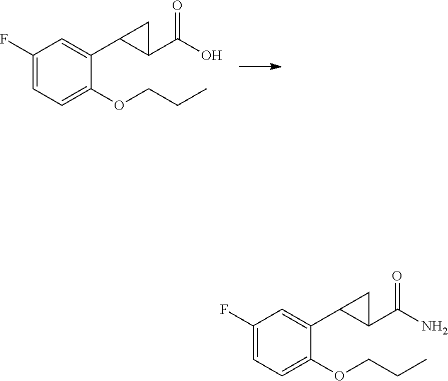 [SMILES: CCCOc1ccc(F)cc1C1CC1C(=O)O ; CCCOc1ccc(F)cc1C1CC1C(N)=O]

To a stirred solution of the acid product from Example 37 in DMF (0.1 M) at room temperature is added 1,1′-carbonyldiimidazole (2.0 equiv). The reaction mixture is allowed to stir at room temperature for 30 min before ammonium acetate (10 equiv) and triethyl amine (5 equiv) are added. The reaction mixture is allowed to stir at room temperature overnight before being quenched by the addition of diethyl ether and 1M HCl. The phases are separated and the aqueous is extracted with diethyl ether (×3). The combined organic extracts are washed with 1M HCl, sat. aq. sodium bicarbonate, water, and brine, and dried over anhydrous sodium sulfate. After filtration, the solvent is removed in vacuo to afford the amide product which is used in the next step without additional purification.

### Example 39

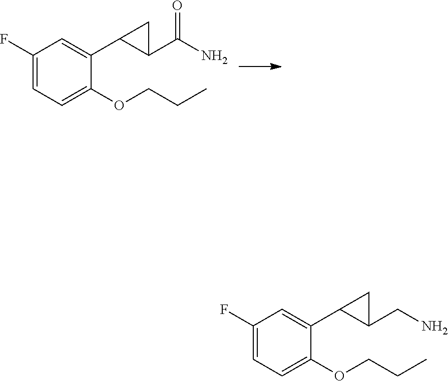 [SMILES: CCCOc1ccc(F)cc1C1CC1C(N)=O ; CCCOc1ccc(F)cc1C1CC1CN]

To a stirred solution of the amide produced in Example 38 in THF (0.1 M) is added dropwise BH₃-THF (1 M solution in THF, 5 equiv). The reaction mixture is refluxed for 4 hours before cooling to room temperature and quenching with methanol. The reaction mixture is concentrated to dryness, diluted with methanol (0.1 M) and 6 M HCl (3 mL), and refluxed for 30 min. After cooling to room temperature, the methanol is removed in vacuo and the crude mixture is basified with 3 M NaOH aqueous solution and extracted with DCM (×5). The combined organic extracts are washed with brine and dried with anhydrous sodium sulfate, filtered, and concentrated in vacuo to afford the target as solid.

### Example 40

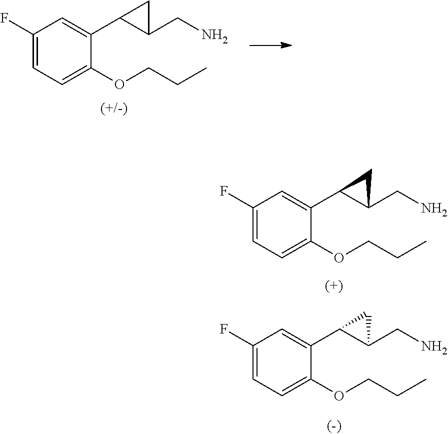 [SMILES: CCCOc1ccc(F)cc1C1CC1CN ; CCCOc1ccc(F)cc1[C@H]1C[C@@H]1CN ; CCCOc1ccc(F)cc1[C@@H]1C[C@H]1CN]

The racemate free base product prepared according to the method of Example 39 is separated by chiral HPLC, hexane/iPrOH/DEA, 90/10 (0.1% diethylamine), 5 μl injection, flowrate of 10 mL/min, λ=280 nm, using Chiralpak AD-H column to afford the (+)-isomer and the (−)-isomer.

### Example 41

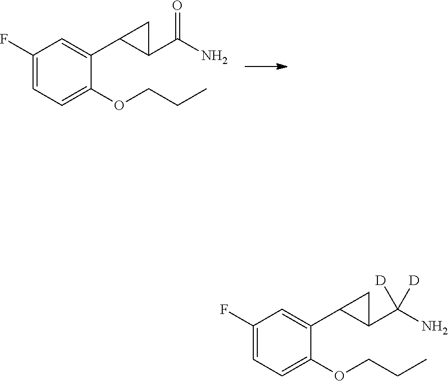 [SMILES: CCCOc1ccc(F)cc1C1CC1C(N)=O ; [2H]C([2H])(N)C1CC1c1cc(F)ccc1OCCC]

To a stirred solution of the amide produced in Example 38 in THF (0.1 M) in an ice bath was added dropwise lithium aluminum deuteride (10 equiv) portion wise. The reaction mixture is allowed to warm to room temperature and additional lithium aluminum deuteride (5 equiv) is added. The reaction mixture was quenched at 0 °C by the addition of water and basified with 3 M KOH solution. The aqueous phase is extracted with DCM (×5) and dried over anhydrous sodium sulfate, filtered, and concentrated in vacuo to afford the d2 free base racemate of the desired product as a white solid.

### Example 42

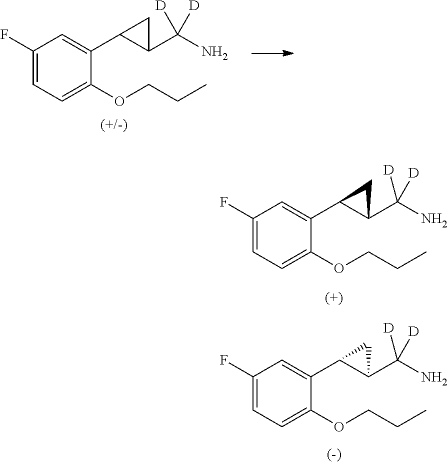 [SMILES: [2H]C([2H])(N)C1CC1c1cc(F)ccc1OCCC ; [2H]C([2H])(N)[C@H]1C[C@@H]1c1cc(F)ccc1OCCC ; [2H]C([2H])(N)[C@@H]1C[C@H]1c1cc(F)ccc1OCCC]

The racemate free base product prepared according to the method of Example 41 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 43

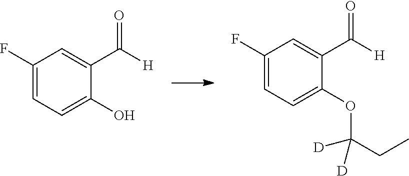

The procedure of Example 34 is repeated, except 1-iodopropane is replaced with 1-iodopropane-1,1-d2 to afford the free base of the target d2 product.

### Example 44

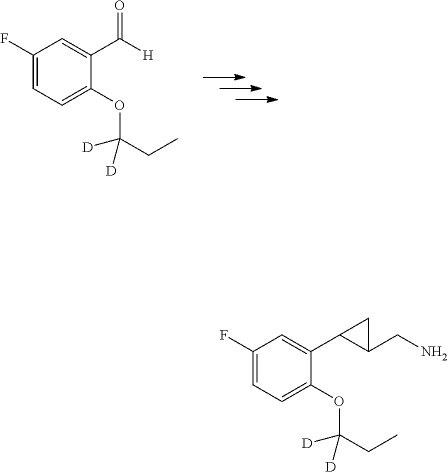 [SMILES: [2H]C([2H])(CC)Oc1ccc(F)cc1C=O ; [2H]C([2H])(CC)Oc1ccc(F)cc1C1CC1CN]

The procedure of Example 35 is repeated, except the aldehyde is replaced with the d2 aldehyde produced according to Example 43. The procedures of Examples 36 through 39 are then followed with the d2 intermediates to afford the free base of the target d2 racemate.

### Example 45

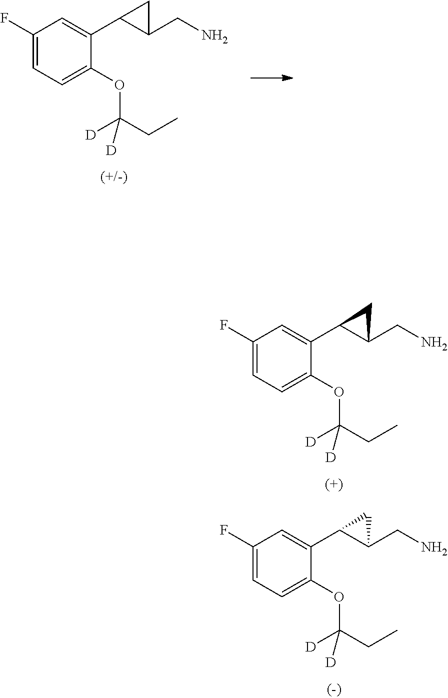 [SMILES: [2H]C([2H])(CC)Oc1ccc(F)cc1C1CC1CN ; [2H]C([2H])(CC)Oc1ccc(F)cc1[C@H]1C[C@@H]1CN ; [2H]C([2H])(CC)Oc1ccc(F)cc1[C@@H]1C[C@H]1CN]

The racemate free base product prepared according to the method of Example 44 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 46

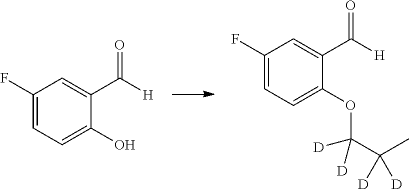 [SMILES: O=Cc1cc(F)ccc1O ; [2H]C([2H])(C)C([2H])([2H])Oc1ccc(F)cc1C=O]

The procedure of Example 34 is repeated, except 2-iodo-1-fluoroethane is replaced with 1-iodopropane-1,1,2,2-d4 to afford the free base of the target d4 aldehyde product.

### Example 47

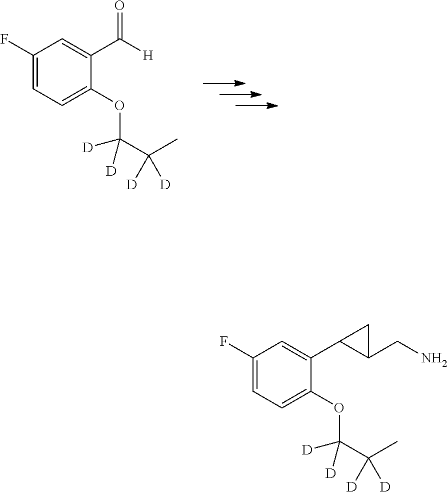 [SMILES: [2H]C([2H])(C)C([2H])([2H])Oc1ccc(F)cc1C=O ; [2H]C([2H])(C)C([2H])([2H])Oc1ccc(F)cc1C1CC1CN]

The procedure of Example 35 is repeated, except the aldehyde is replaced with the d4 aldehyde produced according to Example 46. The procedures of Examples 36 through 39 are then followed with the d4 intermediates to afford the free base of the target d4 racemate.

### Example 48

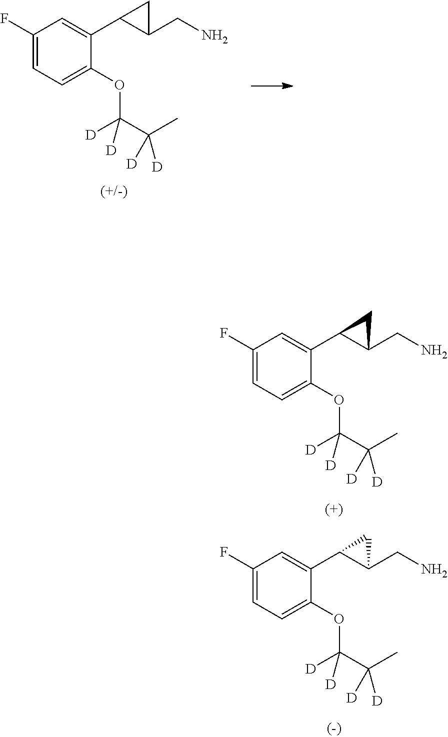 [SMILES: [2H]C([2H])(C)C([2H])([2H])Oc1ccc(F)cc1C1CC1CN ; [2H]C([2H])(C)C([2H])([2H])Oc1ccc(F)cc1[C@H]1C[C@@H]1CN ; [2H]C([2H])(C)C([2H])([2H])Oc1ccc(F)cc1[C@@H]1C[C@H]1CN]

The racemate free base product prepared according to the method of Example 47 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 49

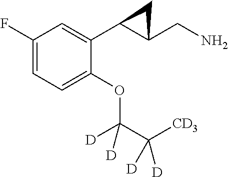 [SMILES: [2H]C([2H])([2H])C([2H])([2H])C([2H])([2H])Oc1ccc(F)cc1[C@H]1C[C@@H]1CN]

The procedures of Examples 46-47 are repeated, except 1-iodopropane-1,1,2,2-d4 was replaced with 1-iodopropane-d7 to afford the target free base product.

### Example 50

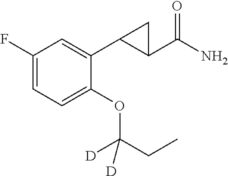 [SMILES: [2H]C([2H])(CC)Oc1ccc(F)cc1C1CC1C(N)=O]

The procedures of Examples 34-38 are repeated, except 1-iodopropane is replaced with 1-iodopropane-1,1-d2 to afford the desired amide.

### Example 51

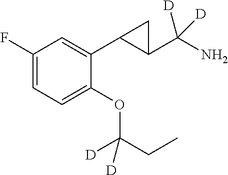 [SMILES: [2H]C([2H])(CC)Oc1ccc(F)cc1C1CC1C([2H])([2H])N]

The procedure of Example 39 is repeated with the amide product of Example 50 to afford the target d6 racemate.

### Example 52

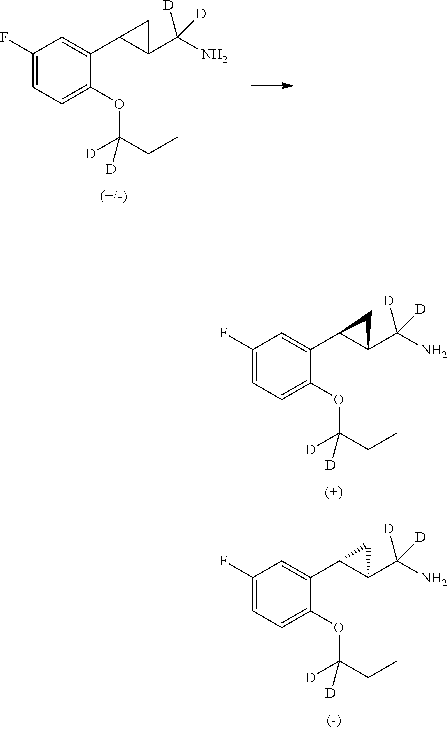 [SMILES: [2H]C([2H])(CC)Oc1ccc(F)cc1C1CC1C([2H])([2H])N ; [2H]C([2H])(CC)Oc1ccc(F)cc1[C@H]1C[C@@H]1C([2H])([2H])N ; [2H]C([2H])(CC)Oc1ccc(F)cc1[C@@H]1C[C@H]1C([2H])([2H])N]

The racemate free base product prepared according to the method of Example 51 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 53

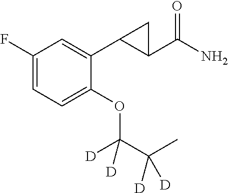 [SMILES: [2H]C([2H])(C)C([2H])([2H])Oc1ccc(F)cc1C1CC1C(N)=O]

The procedures of Examples 34-38 are repeated, except 1-iodopropane is replaced with 1-iodopropane-1,1,2,2-d4 to afford the desired amide.

### Example 54

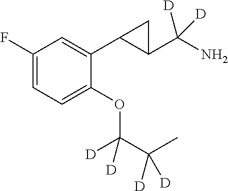 [SMILES: [2H]C([2H])(N)C1CC1c1cc(F)ccc1OC([2H])([2H])C([2H])([2H])C]

The procedures of Example 39 are repeated with the amide product of Example 53 to afford the target d6 racemate.

### Example 55

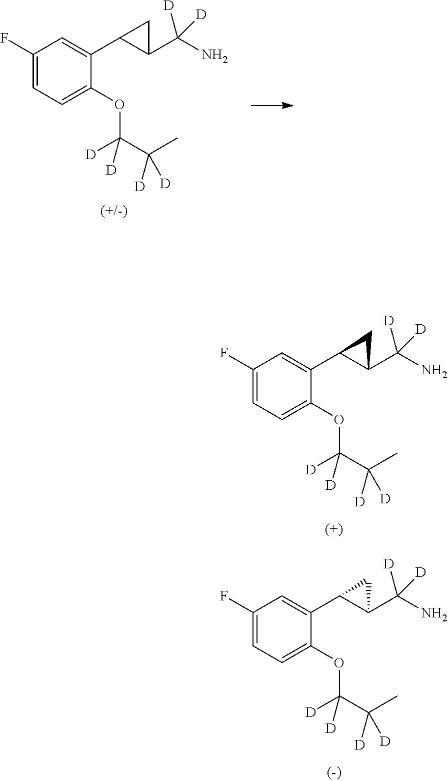 [SMILES: [2H]C([2H])(N)C1CC1c1cc(F)ccc1OC([2H])([2H])C([2H])([2H])C ; [2H]C([2H])(N)[C@H]1C[C@@H]1c1cc(F)ccc1OC([2H])([2H])C([2H])([2H])C ; [2H]C([2H])(N)[C@@H]1C[C@H]1c1cc(F)ccc1OC([2H])([2H])C([2H])([2H])C]

The racemate free base product prepared according to the method of Example 54 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 56

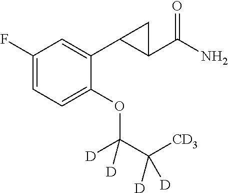 [SMILES: [2H]C([2H])([2H])C([2H])([2H])C([2H])([2H])Oc1ccc(F)cc1C1CC1C(N)=O]

The procedures of Examples 34-38 are repeated, except 1-iodopropane is replaced with 1-iodopropane-d7 to afford the desired amide.

### Example 57

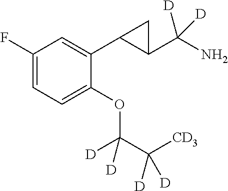 [SMILES: [2H]C([2H])(N)C1CC1c1cc(F)ccc1OC([2H])([2H])C([2H])([2H])C([2H])([2H])[2H]]

The procedures of Example 39 are repeated with the amide product of Example 56 to afford the target d9 racemate.

### Example 58

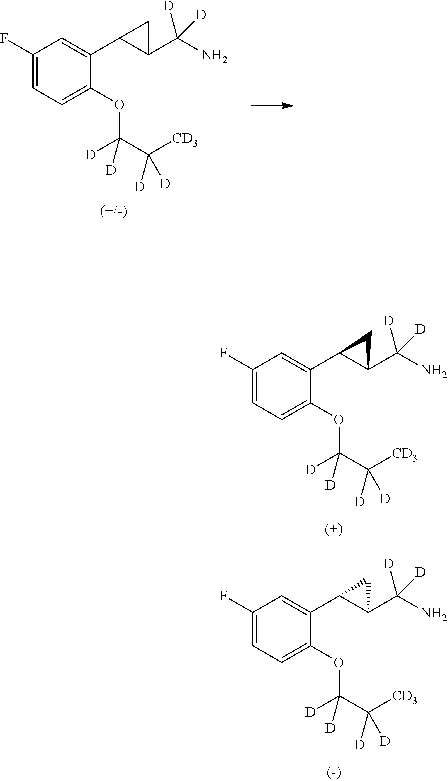 [SMILES: [2H]C([2H])(N)C1CC1c1cc(F)ccc1OC([2H])([2H])C([2H])([2H])C([2H])([2H])[2H] ; [2H]C([2H])(N)[C@H]1C[C@@H]1c1cc(F)ccc1OC([2H])([2H])C([2H])([2H])C([2H])([2H])[2H] ; [2H]C([2H])(N)[C@@H]1C[C@H]1c1cc(F)ccc1OC([2H])([2H])C([2H])([2H])C([2H])([2H])[2H]]

The racemate free base product prepared according to the method of Example 57 is separated by chiral HPLC, hexane/iPrOH/DEA, 90/10 (0.1% diethylamine), 5 μl injection, flowrate of 10 mL/min, λ=280 nm) using Chiralpak AD-H column to afford the (+)-isomer and the (−)-isomer.

### Example 59: Formation of HCl Salt of (+)-isomer of 1-[(1S,2S)-2-(5-fluoro-2-propoxyphenyl)cyclopropyl]methanamine-alpha-d₂ (aka Lumocaserin-alpha-d₂)

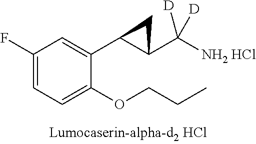

To a solution of the free base (+)-isomer prepared according to the method of Example 42 (400 mg) in methanol (4 mL) is added dropwise 6 M HCl until pH paper indicates pH=1. The volatiles are removed in vacuo and diluted with a minimal amount of methanol. The material is triturated by the addition of dichloromethane (4 mL) followed by hexane (8 mL). The volatiles are removed in vacuo. Toluene is added to the resulting residue and sonicated for 1 min before removal of the solvent in vacuo (×3). The solid is dried to a constant mass to provide the HCl salt product as a white powder.

### Example 60: Formation of HCl Salt of (+)-isomer of 1-[(1S,2S)-2-(5-fluoro-2-propoxyphenyl)cyclopropyl]methanamine-alpha-d₄ (aka Lumocaserin-alpha-d₄)

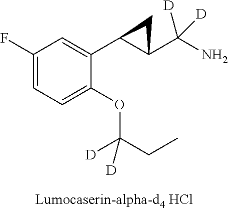 [SMILES: [2H]C([2H])(CC)Oc1ccc(F)cc1[C@H]1C[C@@H]1C([2H])([2H])N]

To a solution of the free base (+)-isomer prepared according to the method of Example 51 (400 mg) in methanol (4 mL) is added dropwise 6 M HCl until pH paper indicates pH=1. The volatiles are removed in vacuo and diluted with a minimal amount of methanol. The material is triturated by the addition of dichloromethane (4 mL) followed by hexane (8 mL). The volatiles are removed in vacuo. Toluene is added to the resulting residue and sonicated for 1 min before removal of the solvent in vacuo (×3). The solid is dried to a constant mass to provide the HCl salt product as a white powder.

Functional Assays for 5-HT_{2A}, 5-HT_{2B}, and 5-HT_{2C} Receptors

To measure 5-HT₂ receptor-mediated Gq activation via Gq/y1 dissociation as measured by Bioluminescence Resonance Energy Transfer (BRET) (McCorvy J D, Wacker D, Wang S, Agegnehu B, Liu J, Lansu K, Tribo A R, Olsen R H J, Che T, Jin J, Roth B L. Structural determinants of 5-HT_{2B} receptor activation and biased agonism. Nat Struct Mo/Biol. 2018; 25(9): 787-96), HEK293T cells were sub-cultured in Dulbecco's Modified Eagle Medium (DMEM) supplemented with 10% dialyzed fetal bovine serum (FBS) and were co-transfected in a 1:1:1:1 ratio with Rluc8-fused human Gaq (Gaq-Rluc8), a GFP2-fused to the C-terminus of human Gy1(Gy1-GFP2), human G131, and 5-HT2 receptor using TransiT-2020. After at least 18-24 hours, transfected cells were plated in poly-lysine coated 96-well white clear bottom cell culture plates in DMEM containing 1% dialyzed FBS at a density of 25,000-40,000 cells in 200 μL per well and incubated overnight. The following day, medium was decanted, and cells were washed with 60 μL of drug buffer (1×HBSS, 20 mM HEPES, pH 7.4), then 60 μL of drug buffer was added per well. Cells were pre-incubated at in a humidified atmosphere at 37 °C before receiving drug stimulation. Drug stimulation utilized 30 μL addition of drug (3X) diluted in McCorvy buffer (1×HBSS, 20 mM HEPES, pH 7.4, supplemented with 0.3% BSA fatty acid free, 0.03% ascorbic acid) and plates were incubated at for 1 hour at 37 °C. Substrate addition occurred 15 minutes before reading and utilized 10 μL of the Rluc substrate coelenterazine 400a for Gq dissociation BRET² (Prolume/Nanolight, 5 μM final concentration). Plates were read for luminescence at 400 nm and fluorescent GFP2 emission at 510 nm at 1 second per well using a Mithras LB940 multimode microplate reader (e.g. one provided by Berthold). The BRET ratios of fluorescence/luminescence were calculated per well and were plotted as a function of drug concentration using Graphpad Prism 8 (Graphpad Software Inc., San Diego, CA). Data were normalized to % 5-HT stimulation and analyzed using nonlinear regression “log(agonist) vs. response” to yield Emax and EC₅₀ parameter estimates. Results are shown in Table 5, where A=<10; B=10 to <100; C=100 to 1000 and D=>1000; +++=>80%; ++=50 to 80%; and +=<50%; NA=No Activity. Gq signaling bias is reported for compounds exhibiting primary signaling for 5-HT2C via Gq-coupled signaling mechanism when compared to signaling mechanisms taking place through β-arrestin. Hallucinogenic (“Hall”) or non-hallucinogenic (“N-Hall”) plastogenic effect is predicted for compounds based on the level 5-HT_{2A} agonist efficacy.

TABLE 5

| | | | | | | | 5- | |
|---|---|---|---|---|---|---|---|---|
| | | | | | | | HT2C | Hall |
| | | | | | | | Gq | or |
| | 5-HT2C | | 5-HT2B | | 5-HT2A | | signal- | N- |
| | EC₅₀ | Emax | EC₅₀ | Emax | EC₅₀ | Emax | ing | Hall |
| | (nM) | (%) | (nM) | (%) | (nM) | (%) | bias? | ? |
| 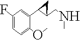 | B | ++ | B | ++ | C | ++ | Yes | N- Hall |
| 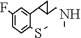 | A | ++ | NA* | NA* | C | ++ | Yes | N- Hall |
| 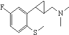 [SMILES: CSc1ccc(F)cc1C1CC1CN(C)C] | B | + | NA* | NA* | D | + | Yes | N- Hall |

Compounds 1 and 2 were also tested in the BRET assay for functional selectivity, with the results set forth below in Table 6:

TABLE 6

| | 5-HT2C | | 5-HT2A | | 5-HT2B | |
|---|---|---|---|---|---|---|
| | EC₅₀ | Emax | EC₅₀ | Emax | EC₅₀ | Emax |
| Compound | (nM) | (%) | (nM) | (%) | (nM) | (%) |
| 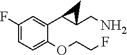 [SMILES: NC[C@H]1C[C@@H]1c1cc(F)ccc1OCCF] | 10.1 | 87 | 1295 | 86 | 191 | 62 |
| Compound 1 | | | | | | |
| 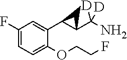 [SMILES: [2H]C([2H])(N)[C@H]1C[C@@H]1c1cc(F)ccc1OCCF] | 9.8 | 90 | 1165 | 92 | 574 | 69 |
| Compound 2 | | | | | | |

Compounds 1 and 2 both demonstrated similar selectivity, efficacy, and potency for the serotonin 5-HT_{2C} and 5-HT_{2A} receptors, with 5-HT_{2C} being the target receptor. Surprisingly, Compound 2 exhibited a greater than 3× reduction in potency at the 5-HT_{2B} receptor when compared to Compound 1. The data suggest that deuteration, e.g., at the carbon atom alpha to the amine group on the Compound 1 scaffold alters the interaction of the resulting compound with the target receptors, a finding which is relevant given that the off-target 5-HT_{2B} receptor is historically associated with cardiac valvulopathy.

Cardiac valvulopathy refers to structural and functional abnormalities of the heart valves—most commonly thickening, fibrosis, and improper leaflet closure—that impair normal blood flow and can lead to regurgitation, heart failure, or arrhythmias. A well-established mechanistic driver of drug-induced valvulopathy is activation of the 5-HT_{2B} receptor expressed on cardiac valvular interstitial cells. Agonism at 5-HT_{2B} stimulates mitogenic and profibrotic signaling pathways, including TGF-β and ERK cascades, promoting pathological proliferation and extracellular matrix deposition within valve tissue. This mechanism has been implicated in the adverse valvular outcomes observed historically with serotonergic agents such as fenfluramine, ergot derivatives, and certain dopamine agonists-underscoring the importance of minimizing 5-HT_{2B} activity in the development of new serotonergic therapeutics. The reduced potency of Compound 2 for the 5-HT_{2B} receptor may therefore provide an additional benefit of reduced side effects when administered clinically.

### CiPA Cardiac Profile Assays

Compound 2 was also tested in CiPA electrophysiological assays (Eurofins) to detect its activity on three ion channel targets.

Sodium Channel Assay: After whole cell configuration was achieved, the cells were held at −80 mV. Onset and steady state block of peak Nav1.5 current was measured using a pulse pattern consisting of a hyperpolarizing pulse to −120 mV for a 200 ms duration, depolarization to −15 mV amplitude for a 40 ms duration, followed by step to 40 mV for 200 ms and finally a 100 ms ramp (1.2 V/s) to a holding potential of −80 mV. Peak current was measured during the step to −15 mV. This paradigm was delivered once every 5s to monitor the current amplitude.

The parameters measured were the difference between the peak inward current on stepping to −15 mV (i.e. peak of the current) and the leak current. All data were filtered for seal quality, seal drop, and current amplitude. The peak current amplitude was calculated before and after compound addition and the amount of block was assessed by dividing the Test compound current amplitude by the Control current amplitude. Control data is the mean Nav1.5 current amplitude collected for three pulses (15 seconds) at the end of the vehicle control application; Test compound data is the mean Nav1.5 current amplitude collected for three pulses (15 seconds) at the end of test concentration application for each concentration.

CiPA Calcium Channel Assay: Onset and steady state block of peak hCav1.2 current was measured using a pulse pattern, repeated every 15 sec. Cells were held at −80 mV for a 50 ms before stepping to −90 mV for 100 ms to measure leak current and then stepped back to −80 mV for 50 ms, depolarization to 0 mV amplitude for a 40 ms duration, followed by step to 40 mV for 200 ms and finally a 100 ms ramp (1.2 V/s) to a holding potential of −80 mV. Peak current was measured during the step to 0 mV. Each concentration was applied for 5 minutes.

The calcium current amplitude was calculated by measuring the difference between the peak inward current on stepping to 0 mV or the peak inward current at the ramp (i.e. peak of the current) and the leak current. The calcium current was assessed in vehicle control conditions and at the end of each five (5) minute compound application.

hERG Potassium Channel Assay:

After whole cell configuration was achieved, the cell is held at −80 mV. The cell is depolarized to +40 mV for 500 ms and then to −80 mV over a 100 ms ramp to elicit the hERG tail current. This paradigm was delivered once every 5s to monitor the current amplitude.

The parameters measured were the maximum tail current evoked on stepping to 40 mV and ramping back to −80 mV from the test pulse. All data were filtered for seal quality, seal drop, and current amplitude. The peak current amplitude was calculated before and after compound addition and the amount of block was assessed by dividing the Test compound current amplitude by the Control current amplitude. Control data was the mean hERG current amplitude collected 15 seconds at the end of the control period; Test compound data was the mean hERG current amplitude collected 15 seconds at the end of each 5 minute test compound application for each concentration.

The results for the testing of Compound 2 (see Table 6 above) in these three assays are set forth below in Table 7:

TABLE 7

| Assay | Compound | IC₅₀ | |
|---|---|---|---|
| Sodium Channel | Compound 2 | >>30 | μM |
| | Tetracaine (control) | 1.45 | μM |
| CiPA Calcium Channel | Compound 2 | >>30 | μM |
| | Nifedipine (control) | 0.41 | μM |
| hERG Potassium Channel | Compound 2 | >>30 | μM |
| | Verapamil (control) | 0.53 | μM |

### Biological Studies

### Head-Twitch Response (HTR) Experiments.

Dose-response studies. Dose-response studies for compounds of Formula I are performed in four consecutive steps:
(a). Formulation work. A suitable (non-toxic) vehicle is identified that can be used to dissolve the compound.
(b). Pilot dose-finding study. HTR-inducing drugs typically have biphasic bell-shaped (inverted U-shaped) dose-response functions, with ascending and descending phases. To quantify the potency of a drug in a HTR dose-response study, doses covering the entire extent of the ascending phase should be included, as well as at least one dose that falls on the descending phase. A pilot dose-finding study is performed to identify a set of doses that matches those requirements. For the pilot, male C57BL/6J mice are injected with a range of doses (typically 0.3-30 mg/kg) by the IP or SC route and then behaviors are recorded in a magnetometer chamber for up to 150 minutes.
(c). Dose-response study. Groups of male C57BL/6J mice with a magnet implant are injected with vehicle or 4-5 doses of the compound (n=5-7 mice/group) by the IP or SC route and then behaviors are recorded in a magnetometer chamber for at least 30 minutes.
(d). Repeated testing. Although potency can typically be quantified based on a single dose-response study, in some instances repeated testing may be necessary. For example, the doses selected for testing may not have been ideal to calculate the median effective dose (ED50 value). If necessary, a second or third dose-response study is performed.

Analysis: The following analyses are performed for dose-response studies:

HTR counts are analyzed using a 1-way ANOVA followed by a post-hoc test (Dunnett's test).

The median effective dose (EDso value) for the compounds (in mg/kg or moles/kg) will be calculated by nonlinear regression using a gaussian or sigmoidal model. The potencies of compounds and other reference compounds can also be compared statistically using an extra-sum-of-squares F-test.

HTR counts can be binned (e.g., blocks of 1, 2, 5, or 10 minutes) and analyzed using a 2-way ANOVA (drug x time) followed by a post-hoc test (Dunnett's test or Tukey's test).

5-HT_{2A} Antagonist blockade studies. Four groups of male C57BL/6J mice with a magnet implant are pretreated SC with the selective 5-HT_{2A} antagonist M100907 (vehicle, 0.001, 0.01, or 0.1 mg/kg). Twenty minutes later, all of the animals are injected IP or SC with one dose of the compound (n=5-7 mice/group) and then behaviors are recorded in a magnetometer chamber for 30 minutes.

5-HT1_{A} Antagonist blockade studies. Four groups of male C57BL/6J mice (n=5-7 mice/group) with a magnet implant are pretreated SC with the selective 5-HT_{1A} antagonist WAY-100635 (vehicle or 1 mg/kg). Twenty minutes later, the animals are injected IP or SC with vehicle or one dose of the compound and then behaviors are recorded in a magnetometer chamber for at least 30 minutes.

Extended time-course studies. Male C57BL/6J mice with a magnet implant are injected IP or SC with up to three different treatments (n=5-6 mice/group) and then behaviors are recorded in a magnetometer chamber for up to 5 hours (the exact assessment period used will depend on the duration-of-action of the Material being tested).

Brain penetration testing. These studies are used to test whether 5-HT2A ligands that do not induce the HTR are brain penetrant in mice. Male C57BL/6J mice with a magnet implant are pretreated IP or SC with vehicle or three doses of the 5-HT2A ligand (n=5-7 mice/group); 20 minutes later, all of the mice are injected IP with 1 mg/kg (±)-DOI HCl, and then behaviors are recorded in a magnetometer chamber for 20-30 minutes.

hERG Inhibition Studies.

All experiments are conducted manually using a HEKA EPC-10 amplifier at room temperature in the whole-cell mode of the patch-clamp technique. HEK293 cells stably expressing hKv11.1 (hERG) under G418 selection can be sourced from the University of Wisconsin, Madison. Cells are cultured in DMEM containing 10% fetal bovine serum, 2 mM glutamine, 1 mM sodium pyruvate, 100 U ml-1 streptomycin, and 500 mg ml-1 penicillin, 100 μg ml-1 G418. The cell line is not authenticated or tested for mycoplasma contamination.

Before experiments, cells are grown to 60-80% confluency, lifted using TrypLE, and plated onto poly-l-lysine-coated coverslips. Patch pipettes are pulled from soda lime glass (micro-haematocrit tubes) and should exhibit resistances of 2-4 MO. For the external solution, normal sodium Ringer is used (160 mM NaCl, 4.5 mM KCl, 2 mM CaCl₂, 1 mM MgCl₂, 10 mM HEPES, pH 7.4 and 290-310 mOsm). The internal solution used is potassium fluoride with ATP (160 mM KF, 2 mM MgCl₂, 10 mM EGTA, 10 mM HEPES, 4 mM NaATP, pH=7.2 and 300-320 mOsm). A two-step pulse (applied every 10 s) from −80 mV initially to 40 mV for 2 s and then to −60 mV for 4 s, is used to elicit hERG currents. The percentage reduction of tail current amplitude by the compounds of Formula I that are tested is determined and data are shown as mean±s.d. (n=3-4 per data point). For all experiments, solutions of the drugs are prepared fresh from 10 mM stocks in DMSO. The final DMSO concentration never exceeds 1%.

### Serotonin and Opioid Receptor Functional Assays.

Functional assay screens at 5-HT and opioid receptors are performed in parallel using the same compound dilutions and 384-well-format high-throughput assay platforms. Assays are used to assess activity at all human isoforms of the receptors, except where noted for the mouse 5-HT_{2A} receptor. Receptor constructs in pcDNA vectors are generated from the Presto-Tango GPCR library39 with minor modifications. All tested compounds of Formula I are serially diluted in drug buffer (HBSS, 20 mM HEPES, pH 7.4 supplemented with 0.1% bovine serum albumin and 0.01% ascorbic acid) and dispensed into 384-well assay plates using a FLIPR Tetra automated dispenser head (Molecular Devices). Every plate includes a positive control such as 5-HT (for all 5-HT receptors), DADLE (DOR), salvinorin A (KOR), and DAMGO (MOR). For measurements of 5-HT_{2A} 5-HT_{2B}, and 5-HT_{2C} Gq-mediated calcium flux function, HEK Flp-In 293, T-Rex stable cell lines (Invitrogen) are loaded with Fluo-4 dye for one hour, stimulated with compounds and read for baseline (0-10 s) and peak fold-over-basal fluorescence (5 min) at 25 °C on the FLIPR Tetra system. For measurement of 5-HT₆ Gs and 5-HT₇ₐ functional assays, -mediated cAMP accumulation is detected using the split-luciferase GloSensor assay in HEKT cells measuring luminescence on a Microbeta Trilux (Perkin Elmer) with a 15 min drug incubation at 25 °C. For 5-HT_{1A}, 5-HT_{1B}, 5-HT_{1F}, MOR, KOR and DOR functional assays, Gi/o, -mediated cAMP inhibition is measured using the split-luciferase GloSensor assay in HEKT cells, conducted similarly to that above, but in combination with either 0.3 μM isoproterenol (5-HT_{1A}, 5-HT_{1B}, 5-HT_{1F}) or 1 μM forskolin (MOR, KOR and DOR) to stimulate endogenous cAMP accumulation. For measurement of 5-HT_{1D}, 5-HT_{1E}, 5-HT₄, and 5-HT₅A functional assays, β-arrestin2 recruitment is measured by the Tango assay using HTLA cells expressing tobacco etch virus (TEV) fused-β-arrestin2, as described previously with minor modifications. Cell lines are not authenticated, but they are purchased mycoplasma-free and tested for mycoplasma contamination. Data for all assays are plotted and nonlinear regression is performed using “log(agonist) vs. response” in GraphPad Prism to yield estimates of the efficacy (Emax and half-maximal effective concentration (EC₅₀)).

### Pharmacokinetic Studies.

Male and female C57/BL6J mice (12 weeks old) are administered a compound of Formula I via i.p. injection at doses of either 50 mg kg-1, 10 mg kg-1 or 1 mg kg-1. Mice are euthanized 15 min or 3 h after injection by cervical dislocation. Two males and two females are used per dose and time point. Brain and liver are collected, flash-frozen in liquid nitrogen, and stored at −80 °C until metabolomic processing. Whole brain and liver sections are lyophilized overnight to complete dryness, then homogenized with 3.2 mm diameter stainless-steel beads using a GenoGrinder for 50 s at 1,500 rpm. Ground tissue is then extracted using 225 μl cold methanol, 190 μl water, 750 μl methyl tert-butyl ether (MTBE). Seven method blanks and seven quality-control samples (pooled human serum, BiolVT) are extracted at the same time as the samples. The nonpolar fraction of MTBE is dried under vacuum and reconstituted in 60 μl of 90:10 (v/v) methanol: toluene containing 1-cyclohexyldodecanoic acid urea as an internal standard. Samples are then vortexed, sonicated and centrifuged before analysis.

For analysis of the tested compound in liver and brain, samples are randomized before injection with method blanks and quality-control samples are analyzed between every ten study samples. A six-point calibration curve is analyzed after column equilibration using blank injections, and then after all study samples. Blanks are injected after the calibration curve to ensure no that none of the tested compound is retained on the column and carried over to samples. Reconstituted sample (5 μl) is injected onto a Waters Acquity UPLC CSH C18 column (100 mm×2.1 mm, 1.7 μm particle size) with an Acquity UPLC CSH C18 VanGuard precolumn (Waters) using a Vanquish UHPLC coupled to a TSQ Altis triple quadrupole mass spectrometer (Thermo Fisher Scientific). Mobile phase A consists of 60:40 v/v acetonitrile/water with 10 mM ammonium formate and 0.1% formic acid. Mobile phase B consists of 90:10 v/v isopropanol/acetonitrile with 10 mM ammonium formate and 0.1% formic acid. Gradients are run from 0-2 min at 15% B; 2-2.5 min 30% B; 2.5-4.5 min 48% B; 4.5-7.3 min 99% B; 7.3-10 min 15% B. The flow rate is 0.600 ml/min and the column is heated to 65 °C. Mass spectrometer conditions are optimized for the target compound by direct infusion. Selected reaction monitoring is performed for the top five ions, with collision energy, source fragmentation, and radiofrequency optimized for the test compound. Data are processed with TraceFinder 4.1 (Thermo Fisher Scientific). Organ weights are recorded. The concentration in the brain is calculated using the experimentally determined number of moles of the target compound in the whole organ divided by the weight of the organ.

### 5-HT Receptor Functional Assays.

Various assays for measuring serotonin receptor activation are known to those of skill in the art, including those methods described in Olsen et al., Nat. Chem. Biol., 2020 August; 16(8):841-49, incorporated herein by reference in its entirety for all purposes. The assays described therein may be utilized to measure the functional activity of any of the serotonin receptor subtypes described herein, including 5-HT₁A, 5-HT₂A, 5-HT_{2B}, and 5-HT_{2C}. In certain embodiments, serotonin (5-hydroxytryptamine) is used as the reference compound.

### Cell Culture

HEK293T cells are maintained, passaged, and transfected in DMEM medium containing 10% FBS, 100 Units/mL penicillin, and 100 μg/mL streptomycin (Gibco-ThermoFisher, Waltham, MA) in a humidified atmosphere at 37 °C and 5% CO₂. After transfection, cells are plated in DMEM containing 1% dialyzed FBS, 100 Units/mL penicillin, and 100 μg/mL streptomycin for BRET2, calcium, and GloSensor assays.

### BRET2 Assays

Cells are plated either in six-well dishes at a density of 700,000-800,000 cells/well, or 10-cm dishes at 7-8 million cells/dish. Cells are transfected 2-4 hours later, using a 1:1:1:1 DNA ratio of receptor: Ga-RLuc8: Gβ:Gy-GFP2 (100 ng/construct for six-well dishes, 750 ng/construct for 10-cm dishes), except for the Gγ-GFP2 screen, where an ethanol co-precipitated mixture of Gβ1-4 is used at twice its normal ratio (1:1:2:1). Transit 2020 (Mirus Biosciences, Madison, WI) is used to complex the DNA at a ratio of 3 μL Transit/μg DNA, in OptiMEM (Gibco-ThermoFisher, Waltham, MA) at a concentration of 10 ng DNA/μL OptiMEM. The next day, cells are harvested from the plate using Versene (0.1M PBS+0.5 mM EDTA, pH 7.4), and plated in poly-D-lysine-coated white, clear bottom 96-well assay plates (Greiner Bio-One, Monroe, NC) at a density of 30,000-50,000 cells/well.

One day after plating in 96-well assay plates, white backings (Perkin Elmer, Waltham, MA) are applied to the plate bottoms, and growth medium is carefully aspirated and replaced immediately with 60 μL of assay buffer (1×HBSS+20 mM HEPES, pH 7.4), followed by a 10 μL addition of freshly prepared 50 μM coelenterazine 400a (Nanolight Technologies, Pinetop, AZ). After a five-minute equilibration period, cells are treated with 30 μL of drug for an additional 5 minutes. Plates are then read in an LB940 Mithras plate reader (Berthold Technologies, Oak Ridge, TN) with 395 nm (RLuc8-coelenterazine 400a) and 510 nm (GFP2) emission filters, at 1 second/well integration times. Plates are read serially six times, and measurements from the sixth read are used in all analyses. BRET2 ratios are computed as the ratio of the GFP2 emission to RLuc8 emission.

### Calcium Mobilization Assays

Cells are plated in 10-cm plates as described in the BRET2 protocol and co-transfected with receptor (1 μg) and Ga-subunit (1 μg) cDNA. The next day, cells are plated at 15,000 cells/well in poly-D-lysine coated black, clear bottom 384-well plates (Greiner Bio-One, Monroe, NC). The following day, growth medium are aspirated and replaced with 20 μL assay buffer containing 1x Fluo-4 Direct Calcium Dye (ThermoFisher Scientific, Waltham, MA) and incubated for 60 minutes at 37 °C (no CO₂). Plates are brought to RT for 10 minutes in the dark before being loaded into a FLIPR Tetra® liquid-handling robot and plate reader (Molecular Devices, San Jose, CA). Baseline fluorescence measurements are taken for 10 seconds followed by robotic drug addition (10 μL) and a 60-second measurement (1 measurement/second). For antagonist assays, cells are first treated with antagonist and kept in the dark at room temperature for ten minutes before agonist addition by the FLIPR Tetra® robot. Maximal response during this time is used to calculate amplitude of the calcium transients. Measurements are analyzed as percentage of maximum signal amplitude for the construct.

Glosensor cAMP Assays

Cells are plated in 10-cm plates as previously described. Cells are transfected with plasmids encoding cDNA for the Glosensor reporter (Promega, Madison, WI), receptor, and Ga-subunit at a ratio of 2:1:1 (2 μg: 1 μg: 1 μg). The next day, cells are plated in black, clear-bottom, 384-well white plates. After aspiration of the medium on the day of the assay, cells are incubated for 60 minutes at 37 °C with 20 μL of 5 mM luciferin substrate (GoldBio, St. Louis, MO) freshly prepared in assay buffer. For Gas activity, 10 μL of drugs are added using the FLIPR Tetra@liquid-handling robot and read after 15 minutes in a Spectramax luminescence plate reader (Molecular Devices, San Jose, CA) with a 0.5 second signal integration time. For Gai activity, 10 μL of drugs are added for a 15-minute incubation period. Subsequently, 10 μL of isoproterenol (final concentration of 200 nM) are added and incubated for an additional 15-minute period before reading.

### Hepatocyte Stability Assay

Purpose: Measure the in vitro metabolic stability of test compound(s) in different species of hepatocytes

Materials and reagents: Compounds of Formula I described herein were selected for study. Control compound verapamil was purchased from Sigma Chemical Co. Cryopreserved Hepatocytes were purchased from BioIVT or RILD and stored at in a liquid nitrogen container. The hepatocytes tested are listed in the table below:

| Species | Strain | Sex | Source |
|---|---|---|---|
| Mouse | ICR/CD-1 | Male | BiolVT |
| Human | N/A | Mixed | BiolVT |

William's E Medium, human recombinant insulin, HEPES, DPBS and GlutaMAX are purchased from Gibco. Isotonic Percoll is purchased from Solarbio. Fetal bovine serum is purchased from Avantor. Dexamethasone is purchased from Solarbio. 96-well plates are purchased from Corning.

Experimental Procedure: Preparation of Working Solutions—10 mM stock solutions of test compound(s) and positive controls in appropriate solvent (DMSO) were prepared. In 96-well plates, diluted to 100 μM by combining 198 μL of 50% acetonitrile/50% water and 2 μL of 10 mM positive controls. Preparation of Hepatocytes—incubation medium (William's E Medium supplemented with GlutaMAX) and hepatocyte thawing medium were placed in a 37 °C water bath, and allowed warming for at least 15 minutes prior to use. A vial of cryopreserved hepatocytes was removed from storage, ensuring that vials remained at cryogenic temperatures until the thawing process ensued. The cells were thawed by placing the vial in a 37 °C water bath and gently shaking the vials for 2 minutes. After thawing was completed, vials were sprayed with 70% ethanol, and the vial was transferred to a biosafety cabinet. A wide-bore pipette tip was used to transfer hepatocytes into 50 mL conical tube containing thawing medium. The 50 mL conical tube was placed into a centrifuge and spin at 80 g for 8 minutes. Upon completion of spin, the thawing medium was aspirated and resuspended hepatocytes in enough incubation medium to yield −1.5×10⁶ cells/mL. Using an AO/PI staining solution, cells were counted and determined the viable cell density. Cells with poor viability (<75% viability) were not acceptable for use. Cells were diluted with incubation medium to a working cell density of 0.5×10⁶ viable cells/mL. Compound Incubation—Pipetted 198 μL of hepatocytes into each well of a 96-well non-coated plate. The plate was placed in the incubator on an orbital shaker to allow the hepatocytes to warm for 10 minutes. 2 μL of the 100 μM test compound(s) or positive control were pipetted into respective wells of the 96-well plate to start the reaction. The final test concentration was 1 μM. The plate was returned to the incubator and placed on an orbital shaker. The reaction for each compound was performed in duplicate. Procedure for Stability Determination −25 μL aliquots of reaction mixture were removed at time points of 0, 15, 30, 60, 90120, and 240 minutes. The aliquots were then mixed with 6 volumes (150 μL) of acetonitrile containing internal standards (IS: 100 nM tolbutamide, 200 nM labetalol, 100 nM ketoprofen, 200 nM imipramine and 200 nM diclofenac) to terminate the reaction. The plate was centrifuged for 45 minutes at 3,220 g. Aliquots of 100 μL of the supernatants were mixed with 100 μL ultrapure water and used for LC/MS/MS analysis.

Data Analysis: All calculations were carried out using Microsoft Excel. Peak areas were determined from extracted ion chromatograms. The in vitro half-life (t_{1/2}) of parent compound was determined by regression analysis of the percent parent disappearance vs. time curve. The in vitro half-life (in vitro t_{1/2}) was determined from the slope value:
in vitro t_{1/2}=−0.693/k
   Conversion of the in vitro t_{1/2} (in min) into the in vitro intrinsic clearance (in vitro CLᵢₙₜ, in μL/min/10⁶ cells) was done using the following equation (mean of duplicate determinations):
in vitro CLᵢₙₜ=kV/N
   Where V=incubation volume (0.2 mL); N=number of hepatocytes per well (0.1×10⁶ cells).

This Hepatocyte Stability assay was conducted on the following compounds of Formula I, with the results set forth in Table 8 below:

TABLE 8

| | | Hepatocyte Stability |
|---|---|---|
| | | (Human Liver Cells) |
| Reference # | Structure | [(% remaining after 4 hrs.)] |
| Compound 1 | 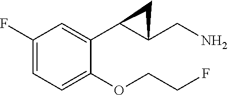 [SMILES: NC[C@H]1C[C@@H]1c1cc(F)ccc1OCCF] | 67% |
| Compound 2 | 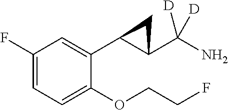 [SMILES: [2H]C([2H])(N)[C@H]1C[C@@H]1c1cc(F)ccc1OCCF] | 97% |
| Compound 3 | 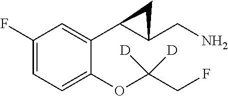 [SMILES: [2H]C([2H])(CF)Oc1ccc(F)cc1[C@H]1C[C@@H]1CN] | 79% |
| Compound 4 | 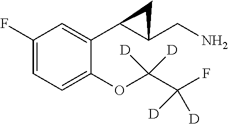 [SMILES: [2H]C([2H])(F)C([2H])([2H])Oc1ccc(F)cc1[C@H]1C[C@@H]1CN] | 79% |
| Compound 5 (Lumocaserin) | 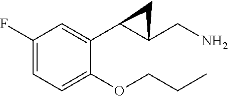 [SMILES: CCCOc1ccc(F)cc1[C@H]1C[C@@H]1CN] | RP |
| Compound 6 (Lumocaserin-alpha-d2) | 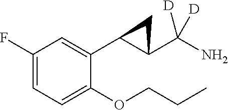 [SMILES: [2H]C([2H])(N)[C@H]1C[C@@H]1c1cc(F)ccc1OCCC] | RP |
| Compound 7 (Lumocaserin-d2) | 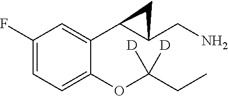 [SMILES: [2H]C([2H])(CC)Oc1ccc(F)cc1[C@H]1C[C@@H]1CN] | RP |
| Compound 8 (Lumocaserin-d4) | 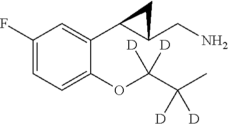 [SMILES: [2H]C([2H])(C)C([2H])([2H])Oc1ccc(F)cc1[C@H]1C[C@@H]1CN] | RP |
| Compound 9 (Lumocaserin-d7) | 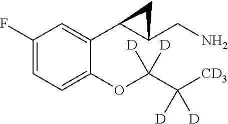 [SMILES: [2H]C([2H])([2H])C([2H])([2H])C([2H])([2H])Oc1ccc(F)cc1[C@H]1C[C@@H]1CN] | RP |
| Compound 10 (Lumocaserin-d9) | 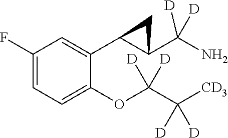 [SMILES: [2H]C([2H])(N)[C@H]1C[C@@H]1c1cc(F)ccc1OC([2H])([2H])C([2H])([2H])C([2H])([2H])[2H]] | RP |
| Compound 11 (Lumocaserin-d6) | 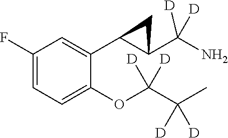 [SMILES: [2H]C([2H])(N)[C@H]1C[C@@H]1c1cc(F)ccc1OC([2H])([2H])C([2H])([2H])C] | RP |
| Compound 12 (Lumocaserin-alpha-d4) | 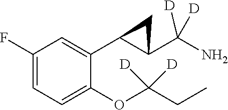 [SMILES: [2H]C([2H])(CC)Oc1ccc(F)cc1[C@H]1C[C@@H]1C([2H])([2H])N] | RP |

*RP = results pending

### Metabolite Identification Assays

Purpose: The objective of this study was to identify the potential metabolites of test compounds and to predict the possible metabolic pathways of test compounds in mouse, rat, dog, monkey and human hepatocytes.

Experimental Procedure: Materials—compounds of Formula I were provided for testing.

Acetonitrile and methanol were purchased from Fisher Scientific. Cryopreserved Hepatocytes were purchased from BiolVT or RILD and stored in a liquid nitrogen container. The hepatocytes used for testing are listed in the table below:

| Species | Strain | Sex | Source |
|---|---|---|---|
| Mouse | ICR/CD-1 | Male | BiolVT |
| Human | N/A | Mixed | BiolVT |

Sample preparation—The stock solution of test compounds was prepared in acetonitrile/DMSO at the concentration of 2 mM. The final test concentration was 10 μM. Hepatocytes thawing medium was prepared by mixing ingredients in table below. The incubation medium was prepared by mixing 49.5 mL Williams E Medium and 0.5 mL glutaMAX. The thawing medium and incubation medium were placed in a 37 °C water bath, then allowed to warm for at least 15 minutes prior to use.

| | | | | | Quantities | |
|---|---|---|---|---|---|---|
| Reagent | Initial Conc. | | Final Conc. | | of Reagents | |
| Williams' Medium E | — | | — | | 31.2 | mL |
| Isotonic Percoll | — | | 30% | | 13.5 | mL |
| DPBS(10×) | — | | — | | 1.5 | mL |
| GlutaMAX ™-1(100×) | 200 mM/100× | | 2 | mM | 500 | μL |
| HEPES | 1M | | 15 | mM | 750 | μL |
| FBS | — | | 5% | | 2.5 | mL |
| Human recombination | 4 | mg/mL | 4 | μg/mL | 50 | μL |
| insulin | | | | | | |
| 10 mM Dexamethasone | 10 | mM | 1 | μM | 5 | μL |

The vial of hepatocytes was removed from storage, ensuring that the vials remained at cryogenic temperatures until the thawing process began. The cells were thawed as quickly as possible by placing the vial in a 37 °C water bath and gently shaking the vial. The vial remained in the water bath until all ice crystals had dissolved and were no longer visible. The vial was opened and the contents were poured into a conical tube containing 50 mL thawing medium, followed by centrifugation at 80×g for 6 minutes. Upon completion of centrifugation, the thawing medium was aspirated and the hepatocytes were resuspended in sufficient incubation medium to yield approximately 1.5×10⁶ cells/mL. Using a Cellometer® Vision instrument, the cells were counted and the viable cell density was determined. Cells with poor viability (<75%) were not used. The cells were diluted with incubation medium to a working density of 1×10⁶ viable cells/mL.

For the 0-minute sample, aliquots (199 μL) of hepatocyte suspension were transferred into a 24-well plate. Subsequently, 600 μL of acetonitrile containing 0.1% formic acid was added into the corresponding wells, followed by the addition of 1 μL of test article working solution (2 mM).

For the 120-minute, 240-minute, and blank (240-minute) samples, the 120-minute and 240-minute incubations were initiated by adding 1 μL of 2 mM compound solution into 24-well plates containing 199 μL of hepatocyte suspension. The blank samples were initiated by adding 1 μL of acetonitrile/DMSO solution into 199 μL of hepatocyte suspension.

The incubations were carried out in an incubator maintained at 37 °C with 5% CO₂ on an orbital shaker at approximately 500 rpm. The reactions were quenched at the designated time points by adding 600 μL of acetonitrile containing 0.1% formic acid into the respective wells.

The supernatants were used for UHPLC-UV-MS/MS analysis. When necessary, 600 μL of supernatant was transferred and evaporated to dryness under a steady stream of nitrogen at room temperature. The dried residues were reconstituted with 100 μL of diluent (acetonitrile:water (v/v)=1:4). If this diluent did not adequately reconstitute the samples, alternative diluents were used. The samples were then centrifuged at 16,000×g for 30 minutes. The resulting supernatants were transferred and analyzed by UHPLC-UV-MS/MS.

The control compound verapamil was included in the experiment using the same incubation method. The time points evaluated were 0 minutes and 240 minutes. The remaining percentage of verapamil was used to monitor hepatocyte activity. The incubation time was compound-dependent and was guided by hepatocyte stability, targeting approximately 50% parent compound remaining relative to the 0-minute sample.

Instrumentation—UHPLC-MS/MS analysis was conducted using a Dionex UltiMate 3000 UHPLC system or a Vanquish UHPLC system (Thermo Scientific, USA). A Thermo Scientific Q Exactive or Orbitrap Exploris 120/240 mass spectrometer (Thermo Scientific, USA) fitted with a HESI probe was used.

Mass Spectrometry Analysis—All analyses were carried out using a high-resolution mass spectrometer. Data were collected in both positive and negative ionization modes. Compound-dependent parameters were optimized for each test article. Parallel reaction monitoring (PRM) was used to acquire MS² spectra. UHPLC-UV was used to collect UV peak areas of the parent drug and metabolites; in cases where product content was too low, UV signals were not observed.

Data Analysis—Metabolite identification was performed by generating metabolites through in vitro experiments, collecting samples at different time points, and analyzing the samples using full-scan MS. Metabolites were identified by comparing ion chromatograms between blank samples and treated samples, taking into account expected metabolites based on predicted gains or losses in molecular mass relative to the parent drug. Once molecular ions corresponding to possible metabolites were detected, they were subjected to MS/MS analysis. The product ion MS/MS spectra of the parent compound were compared with the fragmentation patterns of the proposed metabolite structures. Specific fragment ions exhibiting shifts in m/z values were used to identify the sites of molecular modification.

Data were acquired and processed using Xcalibur software (Thermo Fisher Scientific) and Compound Discoverer (Thermo Fisher Scientific). Relative peak areas were determined from extracted ion chromatograms of hepatocyte samples collected at 240 minutes.

The Metabolite ID assay was conducted on the following compounds of Formula I, with the results set forth in Table 9 below:

TABLE 9

| | | Number of | Nature of |
|---|---|---|---|
| | | Metabolites | Metabolic |
| | | Identified | Conversions |
| | | (Human | (Human |
| Reference # | Structure | Hepatocytes) | Hepatocytes) |
| Compound 1 | 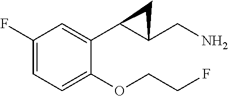 [SMILES: NC[C@H]1C[C@@H]1c1cc(F)ccc1OCCF] | 5 | De-hydrogenation O-Dealkylation Oxidation Hydrogenation Glucuronidation De-amination Methylation |
| Compound 2 | 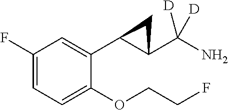 [SMILES: [2H]C([2H])(N)[C@H]1C[C@@H]1c1cc(F)ccc1OCCF] | None | N/A |
| Compound 3 | 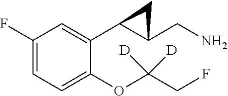 [SMILES: [2H]C([2H])(CF)Oc1ccc(F)cc1[C@H]1C[C@@H]1CN] | 4 | Oxidation Hydrogenation Glucuronidation De-amination Methylation De-hydrogenation |
| Compound 4 | 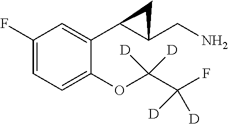 [SMILES: [2H]C([2H])(F)C([2H])([2H])Oc1ccc(F)cc1[C@H]1C[C@@H]1CN] | 4 | Oxidation Hydrogenation Glucuronidation De-amination Methylation De-hydrogenation |
| Compound 5 (Lumocaserin) | 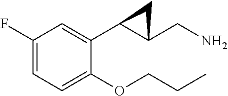 [SMILES: CCCOc1ccc(F)cc1[C@H]1C[C@@H]1CN] | RP | RP |
| Compound 6 (Lumocaserin- alpha-d2) | 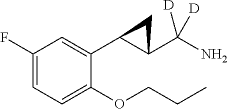 [SMILES: [2H]C([2H])(N)[C@H]1C[C@@H]1c1cc(F)ccc1OCCC] | RP | RP |
| Compound 7 (Lumocaserin- d2) | 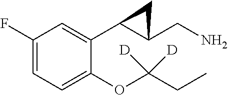 [SMILES: [2H]C([2H])(CC)Oc1ccc(F)cc1[C@H]1C[C@@H]1CN] | RP | RP |
| Compound 8 (Lumocaserin- d4) | 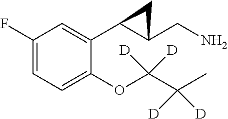 [SMILES: [2H]C([2H])(C)C([2H])([2H])Oc1ccc(F)cc1[C@H]1C[C@@H]1CN] | RP | RP |
| Compound 9 (Lumocaserin- d7) | 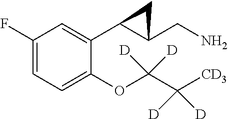 [SMILES: [2H]C([2H])([2H])C([2H])([2H])C([2H])([2H])Oc1ccc(F)cc1[C@H]1C[C@@H]1CN] | RP | RP |
| Compound 10 (Lumocaserin- d9) | 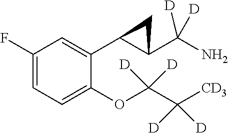 [SMILES: [2H]C([2H])(N)[C@H]1C[C@@H]1c1cc(F)ccc1OC([2H])([2H])C([2H])([2H])C([2H])([2H])[2H]] | RP | RP |
| Compound 11 (Lumocaserin- d6) | 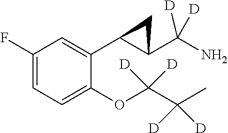 [SMILES: [2H]C([2H])(N)[C@H]1C[C@@H]1c1cc(F)ccc1OC([2H])([2H])C([2H])([2H])C] | RP | RP |
| Compound 12 (Lumocaserin- alpha-d4) | 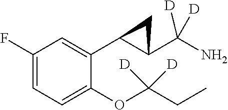 [SMILES: [2H]C([2H])(CC)Oc1ccc(F)cc1[C@H]1C[C@@H]1C([2H])([2H])N] | RP | RP |

*RP = results pending

### 1. Preclinical Study Protocol: Obesity

Title: Evaluation of a Functionally Biased 5-HT_{2C} Receptor Agonist in Combination with Semaglutide for Enhanced Weight Loss in Diet-Induced Obese Mice Study Objective: Demonstrate that co-administration of an orally active, functionally biased 5-HT_{2C} receptor agonist enhances the weight loss, metabolic effects, and preservation of lean body mass (LBM) compared to semaglutide alone in a high-fat diet (HFD)-induced obese mouse model.

### Study Design:

Animal Model: Male C57BL/6J mice, 8-10 weeks old
Diet Induction: Mice are fed a high-fat diet (60% kcal from fat) for 12 weeks to induce obesity prior to treatment.
Randomization: Mice are stratified by baseline body weight and fat mass, then randomized into treatment groups (n=12 per group).
Study Duration: 4 weeks (28 days)

### Treatment Groups:

### Group Treatment

G1 Oral vehicle+SC saline
G2 SC semaglutide+oral vehicle
G3 Oral 5-HT2C agonist+SC saline
G4 Oral 5-HT2C agonist+SC semaglutide

### Dosing Regimens:

5-HT_{2C} Agonist:
   Oral gavage, once daily
   Dose: 3-10 mg/kg (TBD based on prior PK/PD)
   Dosed in the morning to align with active feeding phase
Semaglutide:
   Subcutaneous injection, once weekly
   Dose: 30 nmol/kg SC
   Matched vehicle for control groups

### Endpoints:

### Primary Endpoints:

Percent change in body weight from baseline to Day 28
Change in fat mass (EchoMRI, Day 0 and Day 28)
Change in lean body mass (LBM) from baseline to Day 28 (EchoMRI)

### Secondary Endpoints:

Daily food intake (per cage, normalized per mouse)
Lean mass preservation (EchoMRI)
Glucose tolerance test (GTT) on Day 21
Locomotor activity (Open Field Test, Day 26-27)
Terminal serum levels: leptin, insulin, GLP-1, ghrelin

### Exploratory/Mechanistic Endpoints:

Hypothalamic gene expression (POMC, NPY, AgRP, MC4R by qPCR)
c-Fos immunohistochemistry in hypothalamic nuclei (ARC, PVN, VMH)
Plasma pharmacokinetics (Day 1 and Day 28)

### Data Analysis Plan:

Repeated-measures 2-way ANOVA (treatment×time)
Post hoc Tukey or Sidak correction
Synergy evaluation via Bliss independence or isobologram modeling

### Synergy Criteria:

Combination group must demonstrate statistically significant (p<0.05) weight/fat mass loss greater than the additive effects of the monotherapy groups
Combination group should maintain >75% of lean mass relative to baseline during weight loss

### Expected Outcomes:

Semaglutide and 5-HT2C agonist monotherapy groups show −3-10% weight reduction
Combination group achieves 10% weight loss with improved lean mass retention
Mechanistic endpoints show enhanced hypothalamic activation

### Follow-Up Studies:

Pair-feeding control study to isolate weight loss from appetite suppression
Rebound monitoring post-treatment withdrawal
Extended 8-12 week duration studies for long-term efficacy
   Compliance: This study will be conducted in accordance with IACUC-approved animal care protocols and NIH guidelines for the humane treatment of animals.

### 2. Preclinical Study Protocol: Prader-Willi Syndrome (PWS)

Test whether a pathway-biased 5-HT_{2C} agonist (favoring Gq/PLC over β-arrestin recruitment) reduces pathological food intake and improves compulsive/anxiety-like behaviors in validated PWS models, while demonstrating on-target engagement and a clean behavioral safety profile.

### Models & Groups

### Primary Model (Hyperphagia-Face Valid):

Snord116del/+mouse (well-documented hyperphagia/satiety defects).
Age: 8-14 weeks at start.

### Secondary Model (Social/Affective Features):

Magel2 KO (adds social/anxiety phenotypes, hypothalamic circuitry differences). Arms (per model, per sex): n=12 per group (see power below)
1. WT+Vehicle
2. WT+Test drug (Low)
3. WT+Test drug (High)
4. PWS+Vehicle
5. PWS+Test drug (Low)
6. PWS+Test drug (High)
7. PWS+Active comparator (e.g., lorcaserin or WAY-161503)
8. PWS+On-target block (High test drug+SB-242084, a selective 5-HT_{2C} antagonist)—mechanistic control (subset n=8)
   Optional mechanistic knockout: small 5-HT_{2C}−/− cohort to show loss of efficacy (if available).

### Dosing, PK/PD, and RO Targets

Route: PO gavage (or formulated chow in chronic phase once dose fixed).
Regimens:
   Acute studies: single dose for immediate intake/operant tests.
   Sub-chronic: 14-28 days, QD or BID depending on t % to maintain free brain
   concentrations ~1-3× pathway-bias EC50 during behavioral windows.
PK sampling: satellite mice (n=4/sex/arm) for plasma & brain at T_max, mid-interval, trough on Day 1 and steady-state (Day 7/14).
Exposure targets: if you have in-vitro bias factors (ΔΔlog(τ/KA)), aim for 20-60% estimated central RO at test times; confirm with ex vivo receptor occupancy (e.g., tracer binding) or pS6/c-Fos in ARC/POMC.

### Primary Endpoints (Hyperphagia/Satiety)

1. Fasting-induced refeeding (FIR): 16-h fast→2-h intake (g/kg) and cumulative 24-h intake.
2. Meal pattern analysis (BioDAQ/CLAMS): 24-48 h monitoring for meal size, inter-meal interval, satiety ratio, bout number, lick microstructure (if available).
3. Operant responding for palatable food: fixed ratio (FR5) and progressive ratio (PR) for sucrose pellets (breakpoint).
4. Scheduled limited access (SLA) binge-like intake: daily 1-h access to high-palatable diet; measure escalation slope and total kcal.
5. Novelty-suppressed feeding (NSF): latency to initiate feeding in anxiogenic context (integrates motivation vs anxiety).
   Go/No-go Thresholds (any 2 Achieved without Locomotor Suppression):
≥20-25% reduction in 2-h FIR intake vs PWS+Veh.
Decreased meal size and increased satiety ratio with unchanged total locomotion.
Decreased PR breakpoint (food reinforcement) ≥20%.

### Secondary Endpoints (Anxiety & Compulsivity)

### Anxiety-Like:

Elevated Plus Maze (EPM) and Light-Dark Box (LDB): time in open/bright, entries.
Open Field: center time (with locomotion as covariate).

### Compulsive/Repetitive-Like:

Marble burying (number buried; control for digging latency).
Nestlet shredding (mass % shredded in 30-60 min).
Self-grooming (total time & bout structure/syntaxis).
Touchscreen probabilistic reversal learning (PRL) or set-shifting (IDS/EDS): perseverative errors (compulsivity) vs regressive errors (cognitive flexibility).
Schedule-induced polydipsia (optional index of adjunctive compulsive-like behavior).

### Social/Affective (Magel2 Cohort):

Three-chamber social approach and social novelty indices.
Ultrasonic vocalizations in social context (if lab-set).

### Go/No-go Examples:

↓ perseverative errors on PRL or 1 marble burying ≥20% without anxiogenesis (no decrease in EPM open-arm time).

### Exploratory/Translational Biomarkers

c-Fos/pS6 mapping: ARC (POMC/AgRP), PVN, NAc shell, BNST after acute dosing ±palatable cue.
qPCR/RNAscope: POMC, CART, NPY, AgRP, MC4R; 5-HT_{2C} (Htr2c) editing isoforms.
Plasma hormones: acyl-ghrelin, leptin, insulin; fasting glucose; OGTT at baseline and Day 14/28.
Body comp: EchoMRI % fat, lean.
Sleep/wake (optional): PWS-relevant; piezoelectric monitoring for fragmentation.
Fiber photometry (subset): Ca2+ in POMC neurons during meal initiation to show circuit engagement.

### Behavioral Safety & Specificity

Locomotor activity (Open Field distance) and Rotarod (motor coordination).
Sucrose preference (anhedonia screen).
Startle/PPI (avoid sensorimotor confounds).
5-HT_{2B} liability screen: in vitro counter-profile; in vivo telemetry (PR prolongation) if needed.
On-target validation: SB-242084 pretreatment should attenuate drug effects on hyperphagia endpoints

### Timeline

Week −2 to −1: Habituation, baseline intake, operant shaping, touchscreen training.
Week 0: Acute PK/PD, dose-ranging in WT; select Low/High doses.
Week 1: Acute efficacy: FIR, PR, EPM/LDB with PK linking.
Weeks 2-5: Sub-chronic: daily dosing; BioDAQ meal patterns, SLA, operant PR weekly; EPM/LDB (Week 3), compulsivity battery (Week 4), PRL/set-shifting (Weeks 4-5).
End of study: EchoMRI, OGTT, tissue for c-Fos/gene assays; ex vivo RO.

### Statistics & Power

Primary analyses: two-way or three-way mixed-effects models (Genotype x Treatment x Time), with sex as factor or covariate; Tukey post-hoc.
Multiplicity control: hierarchical—FIR 2-h intake→BioDAQ meal size→PR breakpoint; secondary endpoints FDR-corrected.
Sample size: For a 20-25% reduction in FIR intake (SD=15-20%), a=0.05, 1-P=0.8-n≈10-12/sex/group (inflated for attrition and touchscreen learning drop-outs).
Exclusions/blinding: pre-register criteria (e.g., <70% operant accuracy at baseline); randomize cage-wise; blind dose codes.

### Data Integration & Bias Hypothesis Testing

Pre-specify whether your molecule is G-protein-biased or β-arrestin-biased.
Correlate per-animal exposure and pathway-selective potency (from in-vitro bias factors) with:
   Meal size reductions and satiety ratio (expected Gq-POMC linkage).
   Compulsivity measures (PRL perseveration; may relate to NAc/striatal β-arrestin signaling if that's your mechanistic thesis).
Use partial least squares (PLS) to link multivariate behavioral changes to brain region c-Fos patterns.

### Comparator & Controls

Lorcaserin (historical 5-HT2C reference) or WAY-161503 as efficacy anchors.
mCPP avoided as it's less selective (off-target 2A/2B).
SB-242084 to confirm 5-HT2C mediation.
Pair-feeding control group (yoked to drug-reduced intake) in sub-chronic arm to parse direct anxiolysis/anticompulsivity from secondary effects of weight loss.

### Exemplary Success Criteria

1. Robust, on-target-reversible decreases in hyperphagic intake and improved meal microstructure in Snord116 mice.
2. Improvements in compulsivity/anxiety-like metrics in Magel2 without locomotor or anhedonia liabilities.
3. Exposure-response alignment with pathway bias hypothesis and POMC/ARC engagement.
4. Maintained efficacy over 14 days with favorable metabolic and hormone readouts.

### Prader-Willi Syndrome: Compulsive Behavior Study

Compound 2 (see Table 6 above) was tested in a rodent ethanol operant self-administration model to evaluate the ability of compounds of Formula I to potentially reduce or eliminate compulsive behaviors as a symptom associated with Prader-Willi Syndrome (PWS).

Compulsivity is a core component of the behavioral phenotype of PWS and includes hyperphagia with persistent food preoccupation and food-seeking behaviors, as well as obsessive-compulsive and rigid/repetitive behavioral features. Operant self-administration paradigms provide a quantitative, translational approach to assess reinforcement-driven seeking behavior, wherein the subject performs an instrumental response to obtain a reinforcer. In the addiction and motivational neuroscience literature, operant self-administration is a well-established method for modeling reinforcement, motivational drive, and persistence of reward seeking.

Consistent with clinical and neurobiological evidence that PWS involves dysregulated food motivation/reward processing, changes in operant responding for a reinforcer (including palatable food or drug reinforcers) are interpreted as changes in the reinforcing efficacy and motivational salience of that reinforcer-processes that are directly relevant to compulsive symptom domains in PWS. Accordingly, a compound that reduces operant self-administration and/or reduces motivation to work for a reinforcer provides evidence of reduced compulsive-like reward seeking, supporting therapeutic utility for PWS-associated compulsive phenotypes including hyperphagia and maladaptive reinforcement-seeking behaviors.

### Subjects: Adult Female C57BL/6J Mice

Self-administration protocol: Standard chambers for mouse operant conditioning were used (Med Associates, Inc., St. Albans, VT). The operant boxes were housed in sound-attenuating boxes with exhaust fans. Stimulus events and responses were controlled and monitored using Med PC, Version IV software (Med Associates, Inc., St. Albans, VT). Mice were not food- or water-deprived. Operant self-administration sessions were 20 min and were conducted 5 days per week (Mon-Fri) during the dark period of the light/dark cycle. Mice were placed in the operant box, and the session began by introducing two levers and turning on the house light. Mice were trained to respond on a fixed-ratio 4 (FR4) schedule. Once the subject pressed the active lever 4 times, an infusion pump was activated for 2 seconds to deliver the reinforcer into the well between the two retractable levers. During pump activation, ethanol delivery was cued by turning off the house light and by the presentation of a tone (80 dB) and a stimulus light above the active lever. Responses on the inactive lever had no consequences. At the end of each session, any residual ethanol that was left in the well was measured and the amount was subtracted from the total self-administration volume. This corrected volume was used to calculate ethanol intake (g/kg) during the session. Ethanol intake (g/kg) and number of responses on the active and inactive levers were recorded. During several weeks of alcohol self-administration, mice were injected IP 30 minutes before each daily self-administration session to habituate them to this manipulation. On testing day, mice were randomly assigned into groups to receive vehicle, Compound 2 (25 or 50 mg/kg), or Naltrexone (8 mg/kg) 30 minutes before alcohol self-administration (n=9-13 mice/group).

Results: Data were analyzed using one-way ANOVA with group (vehicle, 25 mg/kg of Compound 2, 50 mg/kg of Compound 2, or 8 mg/kg of Naltrexone) as the main factor. An 8 mg/kg dose of Naltrexone is a standard dose for benchmarking, as this concentration has been previously shown to reliably reduce ethanol intake in rodents without producing sedation or gross motor suppression (see, e.g., Kamdar et al., Psychopharmacology, 2007 June; 192(2):207-17, stating that rodents receiving concentrations greater than 8 mg/kg did not demonstrate a further reduction in alcohol consumption). Data in the graphs are mean±SEM. Analysis of the number of active lever presses indicated a significant effect of group [F (3, 36)=7.922; p<0.001]. Post-hoc comparisons (Dunnett's) indicated that 50 mg/kg of Compound 2 resulted in a significant decrease in responding for alcohol compared to vehicle-treated mice. For ethanol intake (g/kg), the ANOVA also indicated a significant effect [F (3, 36)=7.106; p<0.001]. Post-hoc comparisons indicated that administration of 50 mg/kg of Compound 2 and Naltrexone (8 mg/kg) resulted in a significant reduction in alcohol intake compared to the vehicle group.

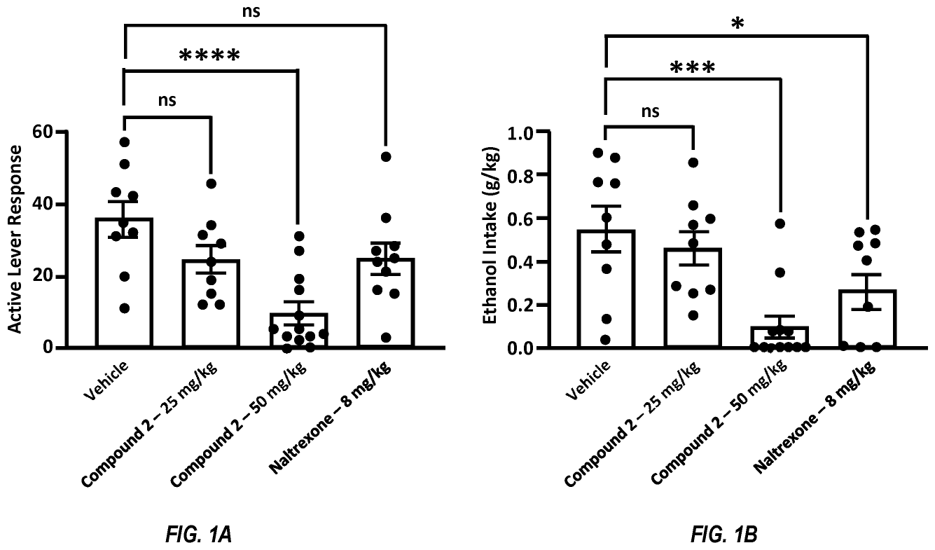

The results of this study were significant at the maximum dose tested of 50 mg/kg, which showed a >75% reduction in active lever presses to access alcohol and a reduction in alcohol intake (volume of ethanol actually consumed) of about 85%. The results are reflected in FIGS. 1A and 1B.

### Evaluation of Compounds in Reducing Drop Attacks and Restoring Brainstem Serotonin Homeostasis in the DBA/2 Mouse Model

### Background & Rationale

DBA/2 mice are genetically predisposed to audiogenic seizures, often progressing to tonic-clonic seizures followed by sudden death or behavioral arrest.
Many DBA/2 seizures begin with loss of postural tone, mimicking drop attacks.
These mice exhibit deficient serotonergic signaling in the dorsal raphe nucleus (DRN) and medullary raphe, resembling the pathophysiology observed in high-risk epilepsy patients.

### Objectives

### +Primary Objectives:

Determine if Test Compound reduces or eliminates drop attacks in DBA/2 mice during audiogenic seizure testing
Assess whether Test Compound restores 5-HT and 5-HIAA levels in the brainstem

### +Secondary Objectives:

Evaluate survival rate post-seizure
Measure changes in c-Fos activation in serotonergic nuclei
Examine postictal respiratory function (if equipment allows)

### Experimental Design

### +Study Arms:

| Group | Treatment | n (per group) |
|---|---|---|
| 1 | Vehicle (saline) | 15 |
| 2 | Test Compound - low dose | 15 |
| 3 | Test Compound - high dose | 15 |
| 4 | Lorcaserin (positive control) | 15 |

Optional: Add a group pre-treated with a 5-HT_{2C} antagonist (SB242084) to confirm target specificity.

### Methodology

### +Animal Preparation:

Male DBA/2 mice (P21-P25, age-matched)
Acclimated for 3 days
Housed under 12:12 light-dark cycle

### +Treatment Protocol:

Once-daily i.p. injection for 5 days
Seizure testing on Day 5, 30 minutes post-dose

### Audiogenic Seizure Testing

Place mouse in sound chamber
Deliver standardized auditory stimulus (e.g., 120 dB siren for 30 seconds)
Video-record behavior

### +Outcome Scoring:

| Behavior | Scoring |
|---|---|
| No response | 0 |
| Wild running | 1 |
| Drop attack (atonia/postural loss) | 2 |
| Tonic-clonic seizure | 3 |
| Seizure-induced death | 4 |

### Neurochemical Analysis

Tissue Collection (1-2 hours post-seizure):
Euthanize animals
Dissect brainstem (pons, medulla)
Analyze:
   Serotonin (5-HT) and 5-HIAA via HPLC
   c-Fos via IHC (in DRN, NTS, and raphe magnus)
   Tph2 mRNA via qPCR (optional)

### Statistical Analysis

One-way ANOVA with post hoc Tukey's test for:
   Drop attack frequency
   Brainstem 5-HT levels
   Survival rates
Correlation between 5-HT levels and behavioral seizure scores
Significance at p<0.05

### Expected Outcomes

Test Compound reduces or abolishes drop attacks
Increases serotonin content in brainstem
Enhances c-Fos activation in serotonergic nuclei
Improves survival post-seizure

### Follow-Up Studies

If successful:
Repeat with chronic dosing model
Use EEG/EMG to confirm atonic seizure characteristics
Test in Scn1a+/− or GAERS models for generalization

### Additional Embodiments

Embodiment 1: A compound of Formula I:

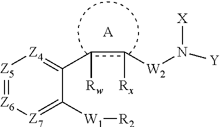

wherein
X and Y are each independently selected from hydrogen, deuterium, optionally substituted C₁-C₈ alkyl, optionally substituted C₂-C₈ alkenyl, and optionally substituted C₂-C₈alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from CF₂, NR₁, O, S, S(O), SO₂, Se, Se(O), and SeO₂;
W₂ is absent or C(R₃)(R_{3′});
Z₄ is selected from N and CR₄;
Z₅ is selected from N and CR₅;
Z₆ is selected from N and CR₆;
Z₇ is selected from N and CR₇;
R₁ is selected from hydrogen, deuterium, optionally substituted C₁-C₈ alkyl, optionally substituted C₂-C₈ alkenyl, —C(O)R₃, —C(O)OR₃, —P(O)(OR₉)₂, —C(O)N(R₉)₂, —SOR₃, and —SO₂R₈;
R₂, R₄, R₅, R₆, and R₇ are each independently selected from hydrogen, deuterium, —N(R₉)₂, —SR₉, halo, optionally substituted C₁-C₈ alkyl, —C₁-C₈ alkoxy, optionally substituted C₂-C₈alkenyl, optionally substituted C₂-C₈ alkynyl, optionally substituted aryl, optionally substituted heteroaryl, hydroxyl, —OC(O)R₈, —OC(O)OR₈, —OP(O)(OR₉)₂, and —OSO₂R₈, or R₂ is a residue selected from Formula III

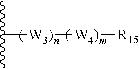

wherein W₃ is, for each occurrence, independently selected from optionally substituted alkylene, optionally substituted arylene, optionally substituted heteroarylene, optionally substituted cycloalkylene, optionally substituted heterocycloalkylene, and

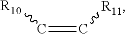

wherein n is an integer selected from 1 to 10,
W₄ is, for each occurrence, independently selected from O, S, Se, C(R₁₂)(R₁₃), optionally substituted arylene, optionally substituted heteroarylene, optionally substituted cycloalkylene, optionally substituted heterocycloalkylene, and

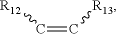

wherein m is an integer selected from 0 to 10, and
R₁₀, R₁₁, R₁₂, R₁₃, and R₁₅ are, for each occurrence, independently selected from optionally substituted C₁-C₈ alkyl, hydrogen, deuterium, halo, and hydroxyl,
or R₄ is taken together with R₅ and the carbons therebetween to form 3- to 7-membered carbocyclic or heterocyclic ring optionally including 1 to 2 additional ring heteromoieties selected from O, S, S(O), SO₂, and NR₉,
or R₅ is taken together with R₆ and the carbons therebetween to form 3- to 7-membered carbocyclic or heterocyclic ring optionally including 1 to 2 additional ring heteromoieties selected from O, S, S(O), SO₂, and NR₉,
or R₆ is taken together with R₇ and the carbons therebetween to form 3- to 7-membered carbocyclic or heterocyclic ring optionally including 1 to 2 additional ring heteromoieties selected from O, S, S(O), SO₂, and NR₉;
R₃ and R_{3′} are each, independently, selected from hydrogen, deuterium, —N(R₉)₂, —SR₉, halo, optionally substituted C₁-C₈ alkyl, —C₁-C₈ alkoxy, and optionally substituted C₂-C₈ alkenyl, or Y is absent and R₃ taken together with carbon to which it is attached and the nitrogen atom to which X is attached form a 3- to 7-membered heterocyclic ring optionally including 1 to 2 additional ring heteromoieties selected from O, S, S(O), SO₂, and NR₉, or R₃ and R_{3′} are taken together with the carbon to which they are attached to form a keto group;
Rₓ and R_{w} are each, independently, absent or selected from hydrogen, deuterium, —N(R₉)₂, —SR₉, halo, optionally substituted C₁-C₈ alkyl, —C₁-C₈ alkoxy, optionally substituted C₂-C₈ alkenyl, and optionally substituted C₂-C₈ alkynyl, or R₂ is taken together with W₁ and the carbon to with Rₓ is attached, and the carbons therebetween, to form a 3- to 7-membered heterocyclic ring optionally including 1 to 2 additional ring heteromoieties selected from O, S, S(O), SO₂, and NR₉;
R₈ is selected from optionally substituted C₁-C₈ alkyl, optionally substituted C₂-C₈ alkenyl, and optionally substituted aryl;
R₉ is independently selected from hydrogen, deuterium, optionally substituted C₁-C₈alkyl, optionally substituted C₂-C₈ alkenyl, and optionally substituted aryl;

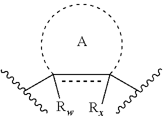

represents a fused ring chosen from an optionally substituted cycloalkanyl, optionally substituted cycloalkenyl, optionally substituted heterocyclyl, optionally substituted aryl, and optionally substituted heteroaryl;
the [symbol] is a single or double bond, wherein Rₓ and R_{w} are absent when it is a double bond;
and salts, solvates, hydrates, prodrugs, and enantiomers thereof.
   Embodiment 2: A compound of Formula I:

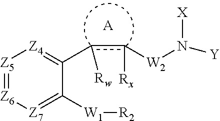

wherein
X and Y are each independently selected from hydrogen, deuterium, optionally substituted C₁-C₈ alkyl, optionally substituted C₂-C₈ alkenyl, and optionally substituted C₂-C₈alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from CF₂, NR₁, O, S, S(O), SO₂, Se, Se(O), and SeO₂;
W₂ is absent or C(R₃)(R_{3′});
Z₄ is selected from N and CR₄;
Z₅ is selected from N and CR₅;
Z₆ is selected from N and CR₆;
Z₇ is selected from N and CR₇;
R₁ is selected from hydrogen, deuterium, optionally substituted C₁-C₈ alkyl, optionally substituted C₂-C₈ alkenyl, —C(O)R₃, —C(O)OR₃, —P(O)(OR₉)₂, —C(O)N(R₉)₂, —SOR₃, and —SO₂R₈;
R₂, R₄, R₅, R₆ and R₇ are each independently selected from hydrogen, deuterium, —N(R₉)₂, —SR₉, halo, optionally substituted C₁-C₈ alkyl, —C₁-C₈ alkoxy, and optionally substituted C₂-C₈ alkenyl, or R₄ is taken together with R₅ and the carbons therebetween to form 3- to 7-membered carbocyclic or heterocyclic ring optionally including 1 to 2 additional ring heteromoieties selected from O, S, S(O), SO₂, and NR₅, or R₅ is taken together with R₆ and the carbons therebetween to form 3- to 7-membered carbocyclic or heterocyclic ring optionally including 1 to 2 additional ring heteromoieties selected from O, S, S(O), SO₂, and NR₉, or R₆ is taken together with R₇ and the carbons therebetween to form 3- to 7-membered carbocyclic or heterocyclic ring optionally including 1 to 2 additional ring heteromoieties selected from O, S, S(O), SO₂, and NR₉;
R₃ and R_{3′} are each, independently, selected from hydrogen, deuterium, —N(R₉)₂, —SR₅, halo, optionally substituted C₁-C₈ alkyl, —C₁-C₈ alkoxy, and optionally substituted C₂-C₈ alkenyl;
Rₓ and R_{w} are each, independently, absent or selected from hydrogen, deuterium, —N(R₉)₂, —SR₉, halo, optionally substituted C₁-C₈ alkyl, —C₁-C₈ alkoxy, and optionally substituted C₂-C₈ alkenyl, or R₂ is taken together with W₁ and the carbon to with Rₓ is attached, and the carbons therebetween, to form a 3- to 7-membered heterocyclic ring optionally including 1 to 2 additional ring heteromoieties selected from O, S, S(O), SO₂, and NR₉;
R₈ is selected from optionally substituted C₁-C₈ alkyl, optionally substituted C₂-C₈ alkenyl, and optionally substituted aryl;
R₉ is independently selected from hydrogen, deuterium, optionally substituted C₁-C₈alkyl, optionally substituted C₂-C₈ alkenyl, and optionally substituted aryl;

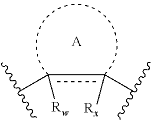

represents a fused ring chosen from an optionally substituted cycloalkanyl, optionally substituted cycloalkenyl, optionally substituted heterocyclyl, optionally substituted aryl, and optionally substituted heteroaryl;
the [symbol] is a single or double bond, wherein Rₓ and R_{w} are absent when it is a double bond,
and salts, solvates, hydrates, prodrugs, and enantiomers thereof.
   Embodiment 3: The compound according to Embodiment 1 or 2, wherein

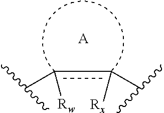

is selected from

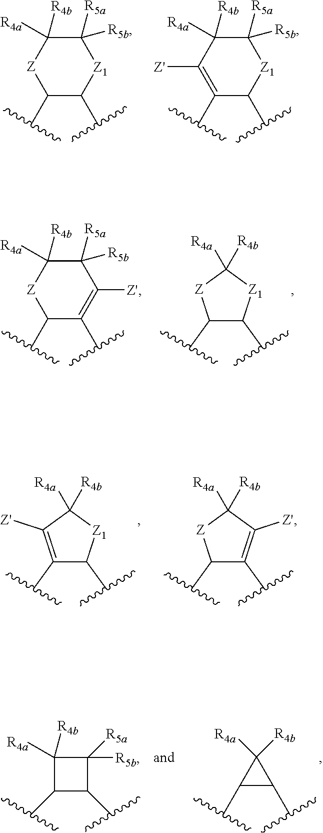

wherein Z and Z₁ are independently selected from O, S and —CZ′₂—, and R₄ₐ, R_{4b}, R₅ₐ, R_{5b} and Z′ are each independently selected from hydrogen, deuterium, halogen, hydroxyl, branched or unbranched C₁-C₈ alkyl, and branched or unbranched C₂-C₈ alkenyl.
Embodiment 4: The compound according to any one of the preceding Embodiments, wherein the [symbol] is a double bond and Rₓ and R_{w} are absent.
Embodiment 5: The compound according to any one of Embodiments 1-3, wherein the [symbol] is a single bond.
Embodiment 6: The compound according to Embodiment 5, wherein R_{w} and Rₓ are hydrogen.
Embodiment 7: The compound according to any one of Embodiments 1-3 and 5, wherein

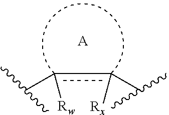

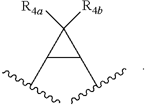

Embodiment 8: The compound according to Embodiment 3, wherein R₄ₐ, R_{4b}, R₅ₐ and R_{5b} are each independently selected from hydrogen, halogen, hydroxyl, deuterium, and C₁-C₄ alkyl.
Embodiment 9: The compound according to any one of the preceding Embodiments, wherein R₂ is selected from optionally substituted C₁-C₄ alkyl and optionally substituted C₂-C₄ alkenyl.
Embodiment 10: The compound according to Embodiment 9, wherein R₂ is substituted with at least one fluorine or at least one deuterium.
Embodiment 11: The compound according to Embodiment 9, wherein R₂ is selected from methyl, ethyl, propyl, and allyl, each of which are optionally substituted with at least one fluorine or at least one deuterium.
Embodiment 12: The compound according to Embodiment 9, wherein R₂ is selected from methyl, CF₃, CD₃, ethyl, fluorinated ethyl, deuterated ethyl, propyl, fluorinated propyl, deuterated propyl, allyl, and deuterated allyl.
Embodiment 13: The compound according to any one of Embodiments 1-8, wherein R₂ is taken together with W₁ and the carbon to with Rₓ is attached, and the carbons therebetween, to form a 3- to 7-membered heterocyclic ring optionally including 1 to 2 additional ring heteromoieties selected from O, S, S(O), SO₂, and NR₉.
Embodiment 14: The compound according to Embodiment 13, wherein the compound of Formula I is selected from compounds of Formula IX:

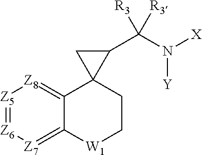

Embodiment 15: The compound according to any one of the preceding Embodiments, wherein at least one of Z₄, Z₅ Z₆ or Z₇ is N.
Embodiment 16: The compound according to Embodiment 15, wherein Z₇ is N.
Embodiment 17: The compound according to any one of Embodiments 1-14, wherein Z₄ is CR₄, Z₅ is CR₅, Z₆ is CR₆, and Z₇ is CR₇.
Embodiment 18: The compound according to Embodiment 17, wherein R₄, R₅ and R₆ are each independently selected from hydrogen and halo.
Embodiment 19: The compound according to any one of Embodiments 17-18, wherein R₅ is fluoro.
Embodiment 20: The compound according to any one of Embodiments 17-19, wherein R₄ is selected from hydrogen, chloro, and fluoro.
Embodiment 21: The compound according to any one of Embodiments 17-20, wherein R₇ is hydrogen.
Embodiment 22: The compound according to any one of the preceding Embodiments, wherein W₂ is C(R₃)(CR_{3′}).
Embodiment 23: The compound according to Embodiment 22, wherein R₃ and R_{3′} are selected from hydrogen and deuterium.
Embodiment 24: The compound according to any one of the preceding Embodiments, wherein X and Y are each independently selected from hydrogen, deuterium, unsubstituted C₁-C₈ alkyl, unsubstituted C₂-C₈ alkenyl, and unsubstituted C₂-C₈ alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉.
Embodiment 25: The compound according to any one of the preceding Embodiments, wherein X and Y are each independently selected from hydrogen, deuterium, and C₁-C₄ alkyl.
Embodiment 26: The compound according to any one of Embodiments 1-24, wherein X and Y are independently selected from hydrogen, deuterium, and -CD₃.
Embodiment 27: The compound according to any one of the preceding Embodiments, wherein X is hydrogen.
Embodiment 28: The compound according to any one of the preceding Embodiments, wherein Y is hydrogen.
Embodiment 29: The compound according to any one of the preceding Embodiments, wherein W₁ is 0.
Embodiment 30: The compound according to any one of Embodiments 1-28, wherein W₁ is selected from Se, Se(O), SeO₂, S, S(O), and SO₂.
Embodiment 31: The compound according to Embodiment 30, wherein W₁ is Se.
Embodiment 32: The compound according to Embodiment 30, wherein W₁ is S.
Embodiment 33: The compound according to any one of Embodiments 1, 3-8, and 15-32, wherein W₃ is an optionally substituted alkylene.
Embodiment 34: The compound according to Embodiment 33, wherein the optionally substituted alkylene is C(R₁₀)(R₁₁).
Embodiment 35: The compound according to any one of Embodiments 1, 3-8, and 15-34, wherein W₄ is an optionally substituted alkylene.
Embodiment 36: The compound according to Embodiment 35, wherein the optionally substituted alkylene is C(R₁₂)(R₁₃).
Embodiment 37: The compound according to any one of Embodiments 1, 3-8, and 15-, wherein at least one of R₁₀, R₁₁, R₁₂, R₁₃ or R₁₅ is deuterium.
Embodiment 38: The compound according to Embodiments 34-37, wherein n is 1, W₃ is C(R₁₀)(R₁₁), and at least one of R₁₀ or R₁₁ is deuterium.
Embodiment 39: The compound according to Embodiment 38, wherein R₁₀ and R₁₁ are both deuterium.
Embodiment 40: The compound according to any one of Embodiments 37-39, wherein R₁₅ is selected from hydrogen, deuterium, and fluoro.
Embodiment 41: The compound according to any one of Embodiments 37-40, wherein m is an integer selected from 0 to 2.
Embodiment 42: The compound according to any one of Embodiments 1, 3-8, 15-40 or 37-41, wherein W₄ is selected from C(R₁₂)(R₁₃) and

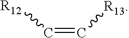

Embodiment 43: The compound according to any one of Embodiments 1, 3-8, or 15-42, wherein R₁₂ and R₁₃ are, for each occurrence, independently selected from hydrogen, deuterium, and fluoro.
Embodiment 44: The compound according to any one of preceding Embodiments 1, 3-13 or 15-43, wherein the compound is selected from compounds of Formula V:

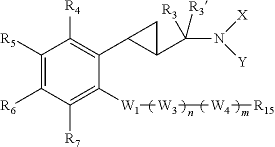

Embodiment 45: The compound according to Embodiment 44, wherein R₃ and R_{3′} are selected from hydrogen and deuterium.
Embodiment 46: The compound according to any one of Embodiments 44-45, wherein X and Y are each hydrogen.
Embodiment 47: The compound according to any one of Embodiments 44-46, wherein at least one of R₅ or R₆ is fluoro.
Embodiment 48: The compound according to any one of Embodiments 44-47, wherein W₃ is C(R₁₀)(R₁₁) and wherein at least one of R₃, R_{3′}, R₁₀ or R₁₁ is deuterium.
Embodiment 49: The compound according to Embodiment 48, wherein n is 1.
Embodiment 50: The compound according to any one of Embodiments 44-49, wherein the compound of Formula V is selected from compounds of Formula VI:

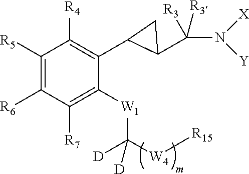

Embodiment 51: The compound according to Embodiment 50, wherein m is an integer selected from 0, 1 and 2 and R₁₅ is selected from hydrogen, deuterium, and fluoro.
Embodiment 52: The compound according to any one of Embodiments 50-51, wherein m is an integer selected from 1 and 2.
Embodiment 53: The compound according to any one of Embodiments 50-52, wherein W₄ is C(R₁₂)(R₁₃) or

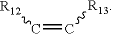

and at least one occurrence of R₁₂ or R₁₃ is fluoro.
Embodiment 54: The compound according to any one of Embodiments 44-53, wherein W₁ is S.
Embodiment 55: The compound according to any one of Embodiments 44-53, wherein W₁ is O.
Embodiment 56: The compound according to any one of Embodiments 50-55, wherein the compound of Formula VI is selected from compounds of Formula V(a):

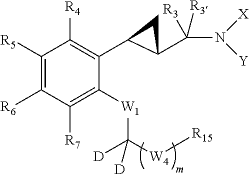

Embodiment 57: A compound selected from Formula VIa:

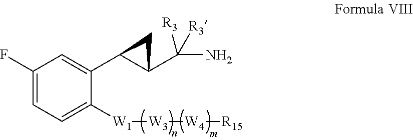

wherein
W₁ is selected from CF₂, NR₁, O, S, S(O), SO₂, Se, Se(O), and SeO₂;
W₃ is, for each occurrence, independently selected from optionally substituted alkylene, optionally substituted arylene, optionally substituted heteroarylene, optionally substituted cycloalkylene, optionally substituted heterocycloalkylene, and

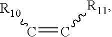

wherein n is an integer selected from 1 to 10;
W₄ is, for each occurrence, independently selected from O, S, Se, optionally substituted alkylene, optionally substituted arylene, optionally substituted heteroarylene, optionally substituted cycloalkylene, optionally substituted heterocycloalkylene, and

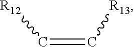

wherein m is an integer selected from 0 to 10;
R₁ is selected from hydrogen, deuterium, optionally substituted C₁-C₈ alkyl, optionally substituted C₂-C₈ alkenyl, —C(O)R₈, —C(O)OR₈, —P(O)(OR₉)₂, —C(O)N(R₉)₂, —SOR₈, and —SO₂R₈;
R₃ and R_{3′} are each independently selected from hydrogen and deuterium; and
R₁₀, R₁₁, R₁₂, R₁₃ and R₁₅ are, for each occurrence, independently selected from optionally substituted C₁-C₈ alkyl, hydrogen, deuterium, halo, and hydroxyl, and
salts, solvates, hydrates, prodrugs, and enantiomers thereof.
   Embodiment 58: The compound according to Embodiment 57, wherein W₃ is an optionally substituted alkylene.
   Embodiment 59: The compound according to Embodiment 58, wherein the optionally substituted alkylene is C(R₁₀)(R₁₁).
   Embodiment 60: The compound according to any one of Embodiments 57-59, wherein W₄ is an optionally substituted alkylene.
   Embodiment 61: The compound according to Embodiment 60, wherein the optionally substituted alkylene is C(R₁₂)(R₁₃).
   Embodiment 62: The compound according to any one of Embodiments 57-61, wherein at least one of R₁₀, R₁₁, R₁₂, R₁₃ or R₁₅ is deuterium.
   Embodiment 63: The compound according to any one of Embodiments 57-62, wherein n is 1, W₃ is C(R₁₀)(R₁₁), and at least one of R₃, R_{3′}, R₁₀ or R₁₁ is deuterium.
   Embodiment 64: The compound according to Embodiment 63, wherein R₁₀ and R₁₁ are both deuterium.
   Embodiment 65: The compound according to any one of Embodiments 57-64, wherein R₃ and R_{3′} are both deuterium.
   Embodiment 66: The compound according to any one of Embodiments 57-65, wherein m is an integer selected from 1 to 3.
   Embodiment 67: The compound according to Embodiment 66, wherein m is 2.
   Embodiment 68: The compound according to any one of Embodiments 57-67, wherein R₁₂ and R₁₃ are, for each occurrence, independently selected from hydrogen, deuterium, and fluoro.
   Embodiment 69: The compound according to Embodiment 57, wherein the compound is selected from:

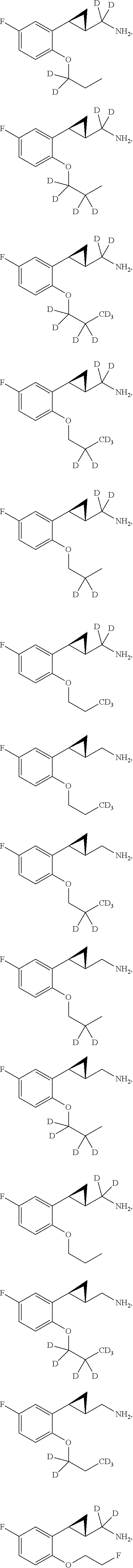

and salts, solvates, hydrates, prodrugs, and enantiomers thereof.
Embodiment 70: The compound according to any one of Embodiments 1-69, wherein the compound comprises a salt.
Embodiment 71: The compound according to any one of Embodiments 1-70, wherein the compound is crystalline.
Embodiment 72: The compound according to any one of Embodiments 1-71, wherein the compound comprises at least one deuterium atom, and wherein the compound exhibits a plasma half life (T_{1/2}) in CB-1 mice that is greater than a plasma half life (T_{1/2}) in CB-1 mice for a comparative compound having the same molecular structure except for the replacement of the at least one deuterium atom with at least one hydrogen atom.
Embodiment 73: The compound according to Embodiment 72, wherein the compound is selected from:

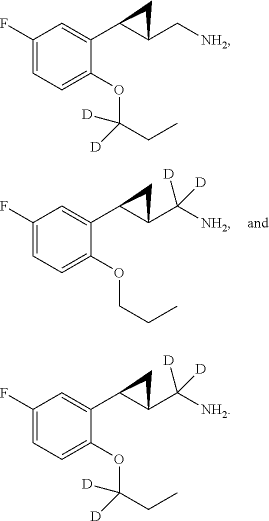

Embodiment 74: The compound according to Embodiment 73, wherein the comparative compound comprises:

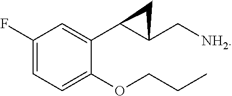 [SMILES: CCCOc1ccc(F)cc1[C@H]1C[C@@H]1CN]

Embodiment 75: The compound according to Embodiment 72, wherein the compound is selected from:

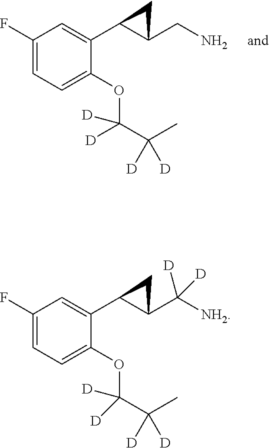 [SMILES: [2H]C([2H])(C)C([2H])([2H])Oc1ccc(F)cc1[C@H]1C[C@@H]1CN ; [2H]C([2H])(N)[C@H]1C[C@@H]1c1cc(F)ccc1OC([2H])([2H])C([2H])([2H])C]

Embodiment 76: The compound according to Embodiment 75, wherein the comparative compound comprises:

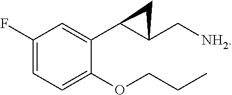 [SMILES: CCCOc1ccc(F)cc1[C@H]1C[C@@H]1CN]

Embodiment 77: The compound according to Embodiment 72, wherein the compound is selected from:

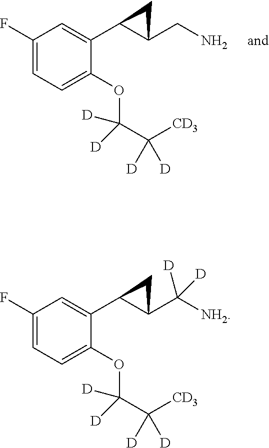 [SMILES: [2H]C([2H])([2H])C([2H])([2H])C([2H])([2H])Oc1ccc(F)cc1[C@H]1C[C@@H]1CN ; [2H]C([2H])(N)[C@H]1C[C@@H]1c1cc(F)ccc1OC([2H])([2H])C([2H])([2H])C([2H])([2H])[2H]]

Embodiment 78: The compound according to any one of Embodiments 72-77, wherein the plasma T_{1/2} of the compound is at least 10% greater than the plasma T_{1/2} of the comparative compound.
Embodiment 79: The compound according to any one of Embodiments 72-77, wherein the plasma T_{1/2} of the compound is at least 20% greater than the plasma T_{1/2} of the comparative compound.
Embodiment 80: The compound according to any one of Embodiments 72-77, wherein the plasma T_{1/2} of the compound is at least 25% greater than the plasma T_{1/2} of the comparative compound.
Embodiment 81: The compound according to any one of Embodiments 72-77, wherein the plasma T_{1/2} of the compound is at least 40% greater than the plasma T_{1/2} of the comparative compound.
Embodiment 82: The compound according to any one of Embodiments 72-77, wherein the plasma T_{1/2} of the compound is at least 50% greater than the plasma T_{1/2} of the comparative compound.
Embodiment 83: The compound according to any one of Embodiments 72-77, wherein the plasma T_{1/2} of the compound is at least two times (2×) greater than the plasma T_{1/2} of the comparative compound.
Embodiment 84: The compound according to any one of Embodiments 72-77, wherein the plasma T_{1/2} of the compound is at least three times (3×) greater than the plasma T₁₂ of the comparative compound.
Embodiment 85: The compound according to any one of Embodiments 72-77, wherein the plasma T_{1/2} of the compound is at least four times (4×) greater than the plasma T₁₂ of the comparative compound.
Embodiment 86: The compound according to any one of Embodiments 72-77, wherein the plasma T_{1/2} of the compound is at least five times (5×) greater than the plasma T_{1/2} of the comparative compound.
Embodiment 87: The compound according to any one of Embodiments 72-77, wherein the plasma T_{1/2} is based on oral dosing of both compounds to CB-1 mice.
Embodiment 88: The compound according to Embodiment 87, wherein both compounds are each separately dosed orally in different mice at about 5 mg/kg.
Embodiment 89: The compound according to Embodiment 87, wherein both compounds are each separately dosed orally in different mice at about 10 mg/kg.
Embodiment 90: A composition comprising, consisting essentially of, or consisting of a compound according to any one of Embodiments 1-89, and a pharmaceutically acceptable carrier.
Embodiment 91: The composition according to Embodiment 90, wherein the composition comprises an oral dosage form.
Embodiment 92: The composition according to Embodiment 91, wherein the oral dosage form comprises a tablet or capsule.
Embodiment 93: A method of treating or preventing a disease or disorder, comprising administering a pharmaceutically acceptable amount of a compound or composition according to any of the preceding Embodiments to a subject in need thereof.
Embodiment 94: The method according to Embodiment 93, wherein the disease or disorder is a psychological disease or disorder.
Embodiment 95: The method according to any one of Embodiments 93-94, wherein the disease or disorder can be treated or prevented by modulation of 5-HT_{2C} receptors.
Embodiment 96: The method according to Embodiment 95, wherein the compound is a 5-HT_{2C} receptor agonist.
Embodiment 97: The method according to any one of Embodiments 93-96, wherein the compound is a 5-HT_{2C} receptor agonist that induces a G_{q}-mediated signaling bias when compared to β-arrestin-mediated signaling.
Embodiment 98: The method according to any one of Embodiments 93-97, wherein the disease or disorder is selected from a psychotic disorder, seizures, obesity, gastrointestinal disorder, sleep apnea, hypertension, hyperlipidemia, a cardiovascular disease, dementia, memory deficit, mild cognitive impairment, Parkinson's Disease, Alzheimer's Disease, an intellectual deficit associated with Alzheimer's disease, Huntington's Disease, dyskinesia, chronic pain, migraine, epilepsy, abuse or addiction to alcohol and drugs, and sexual dysfunction in males or females.
Embodiment 99: The method according to any one of Embodiments 93-98, wherein the compound or composition is contained in an oral dosage form.
Embodiment 100: The method according to Embodiment 99, wherein the oral dosage form is administered once or twice daily.
Embodiment 101: The method according to Embodiment 99, wherein the oral dosage form is administered once daily.
Embodiment 102: The method according to any one of Embodiments 93-101, wherein the subject is administered from about 5 mg to about 250 mg of the compound per day.
Embodiment 103: The method according to any one of Embodiments 93-101, wherein the subject is administered from about 20 mg to about 200 mg of the compound per day.
Embodiment 104: The method according to any one of Embodiments 93-101, wherein the subject is administered from about 40 mg to about 150 mg of the compound per day.
Embodiment 105: The method according to any one of Embodiments 93-104, wherein the compound is administered to the subject in an amount of from about 0.1 mg/kg to about 10 mg/kg per day.
Embodiment 106: The method according to any one of Embodiments 93-104, wherein the compound is administered to the subject in an amount of from about 0.25 mg/kg to about 5 mg/kg per day.
Embodiment 107: The method according to any one of Embodiments 93-104, wherein the compound is administered to the subject in an amount of from about 0.5 mg/kg to about 2.5 mg/kg per day.
Embodiment 108: The method according to any one of Embodiments 99-107, wherein the oral dosage form comprises a solid capsule or solid tablet.
Embodiment 109: The method according to any one of Embodiments 93-100 and 102-108, wherein the subject is administered from about 40 mg to about 80 mg of the compound in a solid capsule or solid tablet twice per day.
Embodiment 110: The method according to any one of Embodiments 93-100 and 102-108, wherein the subject is administered from about 40 mg to about 80 mg of the compound twice per day.
Embodiment 111: The method according to any one of Embodiments 93-100 and 102-108, wherein the subject is administered from about 25 mg to about 100 mg of the compound twice per day.
Embodiment 112: The method according to any one of Embodiments 93-111, wherein the compound is administered with food.
Embodiment 113: The method according to any one of Embodiments 93-111, wherein the compound is administered to the subject when the subject has at least some food in the subject's stomach.
Embodiment 114: The method according to any one of Embodiments 93-113, wherein the subject is administered a dose of from about 0.25 mg/kg to about 2.50 mg/kg once or twice per day.
Embodiment 115: The method according to any one of Embodiments 93-113, wherein the subject is administered a dose of from about 0.25 mg/kg to about 2.50 mg/kg twice per day.
Embodiment 116: The method according to any one of Embodiments 93-115, wherein the disease or disorder is obesity.
Embodiment 117: The method according to any one of Embodiments 93-115, wherein the disease or disorder is a disease or disorder associated with seizures.
Embodiment 118: The method according to Embodiment 117, wherein the disease or disorder is a Developmental Epileptic Encephalopathy.
Embodiment 119: The method according to Embodiment 117, wherein the disease or disorder is Dravet Syndrome.
Embodiment 120: The method according to Embodiment 117, wherein the disease or disorder is Lennox Gastaut Syndrome.
Embodiment 121: The method according to Embodiment 117, wherein the disease or disorder is Tuberous sclerosis.
Embodiment 122: The method according to Embodiment 117, wherein the disease or disorder is CDKL5 deficiency disorder.
Embodiment 123: The method according to Embodiment 117, wherein the disease or disorder is associated with absence seizures.
Embodiment 124: The method according to Embodiment 123, wherein the absence seizures are associated with or without myoclonia.
Embodiment 125: The method according to Embodiment 117, wherein the disease or disorder is West Syndrome.
Embodiment 126: The method according to Embodiment 117, wherein the disease or disorder is Ohtahara Syndrome.
Embodiment 127: The method according to Embodiment 117, wherein the disease or disorder is Doose Syndrome.
Embodiment 128: The method according to Embodiment 117, wherein the disease or disorder is Epilepsy of Infancy with Migrating Focal Seizures (EIMFS).
Embodiment 129: The method according to Embodiment 117, wherein the disease or disorder is CDKL5 Deficiency Disorder.
Embodiment 130: The method according to Embodiment 117, wherein the disease or disorder is PCDH19-Related Epilepsy.
Embodiment 131: The method according to Embodiment 117, wherein the disease or disorder is Rett Syndrome.
Embodiment 132: The method according to Embodiment 117, wherein the disease or disorder is Pyridoxine-Dependent Epilepsy (PDE).
Embodiment 133: The method according to Embodiment 117, wherein the disease or disorder is a SCN8A-Related Epilepsy.
Embodiment 134: The method according to Embodiment 117, wherein the disease or disorder is GLUT1 Deficiency Syndrome.
Embodiment 135: The method according to Embodiment 117, wherein the disease or disorder is KCNQ2 Encephalopathy.
Embodiment 136: The method according to Embodiment 117, wherein the disease or disorder is STXBP1 Encephalopathy.
Embodiment 137: The method according to Embodiment 117, wherein the disease or disorder is a neurodevelopmental disorder associated with one or more mutations of the TCF4 gene.
Embodiment 138: The method according to Embodiment 137, wherein the disease or disorder is associated with intellectual disabilities.
Embodiment 139: The method according to Embodiment 137 or 138, wherein the disease or disorder is Pitt-Hopkins Syndrome (PTHS).
Embodiment 140: The method according to Embodiment 117, wherein the disease or disorder is associated with the dysfunction of at least one gene selected from CACNA1H, CACNA1A, GABRB3, GABRG2, SCN1A, SCN2A, SCC2A1, and CHRNA4.
Embodiment 141: The method according to Embodiment 117 or 140, wherein the disease or disorder is associated with polygenic mutations.
Embodiment 142: The method according to any one of Embodiments 117 and 140-141, wherein the disease or disorder is absence seizures.
Embodiment 143: The method according to Embodiment 142, wherein the disease or disorder is treatment-resistant absence seizures.
Embodiment 144: The method according to Embodiment 143, wherein the treatment-resistant absence seizures are associated with dysfunction of at least one gene selected from CACNA1H and GABRB3.
Embodiment 145: The method according to Embodiment 117, wherein the disease or disorder is associated with the dysfunction of at least one gene selected from CACNA1H, GABRB3, SCN1A, and TCF4.
Embodiment 146: The method according to Embodiment 145, wherein the disease or disorder is selected from Lennox-Gastaut Syndrome and Dravet Syndrome.
Embodiment 147: The method according to Embodiment 145, wherein the disease or disorder is selected from absence seizures and Pitt-Hopkins Syndrome.
Embodiment 148: The method according to any one of Embodiments 145-147, wherein the subject has one or more single nucleotide polymorphisms in a gene.
Embodiment 149: The method according to any one of Embodiments 145-147, wherein the subject has a chromosomal deletion including at least a portion of a gene.
Embodiment 150: The method according to any one of Embodiments 145-147, wherein the subject has a complete deletion of a gene.
Embodiment 151: The method according to any one of Embodiments 145-147, wherein the subject has a chromosomal translocation comprising at least a portion of a gene.
Embodiment 152: The method according to any one of Embodiments 145-147, wherein the subject has a translocation, frameshift, or non-sense mutation in a gene.
Embodiment 153: The method according to any one of Embodiments 145-152, wherein the gene is the TCF4 gene.
Embodiment 154: The method according to any one of Embodiments 145-152, wherein the gene is the SCN1A gene.
Embodiment 155: The method according to any one of Embodiments 145-152, wherein the gene is the GABRB3.
Embodiment 156: The method according to any one of Embodiments 145-152, wherein the gene is the CACNA1H.
Embodiment 157: The method according to any one of Embodiments 93-156, wherein the subject is an infant or pediatric subject.
Embodiment 158: The method according to any one of Embodiments 93-157, wherein the subject has an age selected from about 16 years of age or less, about 12 years of age or less, about 8 years of age or less, about 5 years of age or less, and about 2 years of age or less.
Embodiment 159: The method according to any one of Embodiments 93-156, wherein the subject is an adult subject.
Embodiment 160: The method according to Embodiment 159, wherein the adult subject is at least 18 years old.
Embodiment 161: A method of treating seizures in a subject exhibiting dysfunction with one or more genes selected from CACNA1H, CACNA1A, GABRB3, GABRG2, SCN1A, SCN2A, SCC2A1, CHRNA4, and TCF4, the method comprising administering to a subject a therapeutically effective amount of the compound according to any one of Embodiments 1-92.
Embodiment 162: The method according to any one of Embodiments 93-115, wherein the disease or disorder is a psychotic disorder.
Embodiment 163: The method according to Embodiment 162, wherein the psychotic disorder is schizophrenia.
Embodiment 164: The method according to any one of Embodiments 93-97 and 99-115, wherein the disease or disorder is Prader-Willi Syndrome (PWS).
Embodiment 165: The method according to Embodiment 164, wherein the subject in need thereof has a genetically confirmed diagnosis of Prader-Willi syndrome comprising loss of expression of paternally expressed genes within the 15q11-q13 chromosomal region.
Embodiment 166: The method according to Embodiment 164, wherein the Prader-Willi Syndrome is associated with a genetic defect selected from: a paternal deletion of the 15q11-q13 region; maternal uniparental disomy of chromosome 15; and an imprinting defect resulting in loss of paternal gene expression within the 15q11-q13 locus.
Embodiment 167: The method of any one of embodiments 164-166, wherein administering the at least one 5-HT_{2C} receptor agonist to the subject in need thereof reduces or eliminates at least one symptom associated with Prader-Willi Syndrome.
Embodiment 168: The method of embodiment 167, wherein the at least one symptom is selected from: difficulty in sucking, difficulty in feeding, poor muscle tone, growth hormone deficiency, low levels of sex hormones, a constant feeling of hunger, hyperphagia, weight gain, obesity, short stature, poor motor skills, underdeveloped sex organs, intellectual disability, learning disability, delayed speech development, delayed language development, infertility, cognitive rigidity, cognitive impairment, emotional lability, obsessive-compulsive behavior, aggression, a compulsive behavior, hyperactivity, cognitive impairment, bipolar disorder with psychosis, excessive daytime sleepiness, attention deficits, scoliosis, osteopenia/osteoporosis, gastrointestinal motility, sleep disturbances, stress or anxiety, and reduced pain sensitivity.
Embodiment 169: The method of Embodiment 168, wherein the at least one symptom is selected from a compulsive behavior, disturbances in REM sleep, anxiety, aggression, hyperphagia, and a constant feeling of hunger.
Embodiment 170: The method of Embodiment 168, wherein administering the at least one 5-HT_{2C} receptor agonist to the subject in need thereof reduces or eliminates at least two symptoms associated with Prader-Willi Syndrome.
Embodiment 171: The method of Embodiment 168, wherein administering the at least one 5-HT_{2C} receptor agonist reduces two or more symptoms associated with Prader-Willi Syndrome, wherein the two or more symptoms are selected from a compulsive behavior, disturbances in REM sleep, anxiety, aggression, hyperphagia, and a constant feeling of hunger.
Embodiment 172: The method of Embodiment 168, wherein administering the at least one 5-HT_{2C} receptor agonist reduces compulsive behavior and hyperphagia associated with Prader-Willi Syndrome.
Embodiment 173: The method according to any one of Embodiments 171-172, wherein the compulsive behavior comprises drug or alcohol use.
Embodiment 174: The method according to any one of Embodiments 93-173, wherein the subject is not being administered a contraindicated secondary drug.
Embodiment 175: The method according to Embodiment 174, wherein the secondary contraindicated drug is selected from at least one of a serotonin receptor agonist, a serotonin receptor antagonist, a selective norepinephrine reuptake inhibitor (SNRI), a selective serotonin reuptake inhibitor (SSRI), and a monoamine oxidase inhibitor.
Embodiment 176: A method of treating at least one symptom of Prader-Willi syndrome in a subject having reduced or absent expression of one or more paternally expressed genes within the 15q11-q13 chromosomal region, the method comprising administering to the subject a therapeutically effective amount of at least one compound of Formula VI:

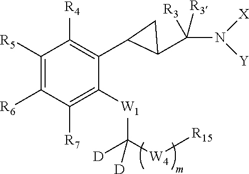

wherein
X and Y are each independently selected from hydrogen, deuterium, optionally substituted C₁-C₈ alkyl, optionally substituted C₂-C₈ alkenyl, and optionally substituted C₂-C₈ alkynyl, or Y is taken together with X and the nitrogen atom therebetween to form a 3- to 7-membered heterocyclic ring optionally including 1 to 2 additional ring heteromoieties selected from O, S, S(O), SO₂, and NRq;
W₁ is selected from O, S, S(O), SO₂, Se, Se(O), and SeO₂;
W₄ is, for each occurrence, independently selected from O, S, Se, C(R₁₂)(R₁₃), optionally substituted arylene, optionally substituted heteroarylene, optionally substituted cycloalkylene, optionally substituted heterocycloalkylene, and

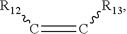

m is an integer selected from 0 to 10;
R₁₂, R₁₃ and R₁₅ are, for each occurrence, independently selected from hydrogen, deuterium, halo, and hydroxyl;
R₄, R₅, R₆ and R₇ are each independently selected from hydrogen, deuterium, —N(R₉)₂, —SR₉, halo, optionally substituted C₁-C₈ alkyl, —C₁-C₃ alkoxy, optionally substituted C₂-C₈ alkenyl, optionally substituted C₂-C₈ alkynyl, optionally substituted aryl, optionally substituted heteroaryl, hydroxyl, —OC(O)R₃, —OC(O)OR₃, —OP(O)(OR₉)₂, and —OSO₂R₃,
or R₄ is taken together with R₅ and the carbons therebetween to form 3- to 7-membered carbocyclic or heterocyclic ring optionally including 1 to 2 additional ring heteromoieties selected from O, S, S(O), SO₂, and NR₉,
or R₅ is taken together with R₆ and the carbons therebetween to form 3- to 7-membered carbocyclic or heterocyclic ring optionally including 1 to 2 additional ring heteromoieties selected from O, S, S(O), SO₂, and NR₉,
or R₆ is taken together with R₇ and the carbons therebetween to form 3- to 7-membered carbocyclic or heterocyclic ring optionally including 1 to 2 additional ring heteromoieties selected from O, S, S(O), SO₂, and NRq;
R₃ and R_{3′} are each, independently, selected from hydrogen, deuterium, —N(R₉)₂, —SR₅, halo, optionally substituted C₁-C₈ alkyl, —C₁-C₈ alkoxy, and optionally substituted C₂-C₈ alkenyl;
R₈ is selected from optionally substituted C₁-C₈ alkyl, optionally substituted C₂-C₈ alkenyl, and optionally substituted aryl;
R₉ is independently selected from hydrogen, deuterium, optionally substituted C₁-C₈alkyl, optionally substituted C₂-C₈ alkenyl, and optionally substituted aryl;
and salts, solvates, hydrates, prodrugs, and enantiomers thereof.
   Embodiment 177: The method according to Embodiment 176, wherein R₄, R₅, R₆ and R₇ are each independently selected from hydrogen, deuterium, —N(R₉)₂, —SR₉, halo, optionally substituted C₁-C₈ alkyl, —C₁-C₈ alkoxy, optionally substituted C₂-C₈ alkenyl, optionally substituted C₂-C₈ alkynyl, optionally substituted aryl, and optionally substituted heteroaryl.
   Embodiment 178: The method according to any one of Embodiments 176-177, wherein X and Y are hydrogen.
   Embodiment 179: The method according to any one of Embodiments 176-178, wherein W₁ is selected from S and Se.
   Embodiment 180: The method according to any one of Embodiments 176-178, wherein W₁ is 0.
   Embodiment 181: The method according to any one of Embodiments 176-180, wherein R₃ and R_{3′} are selected from hydrogen and deuterium.
   Embodiment 182: The method according to any one of Embodiments 176-181, wherein R₄, R₅, R₆ and R₇ are selected from hydrogen, deuterium, and halo.
   Embodiment 183: The method according to any one of Embodiments 176-182, wherein R₅ is fluoro.
   Embodiment 184: The method according to any one of Embodiments 176-183, wherein R₄, R₆ and R₇ are each hydrogen.
   Embodiment 185: The method according to Embodiment 184, wherein W₁ is selected from S and Se.
   Embodiment 186: The method according to Embodiment 184, wherein W₁ is O.
   Embodiment 187: The method according to any one of Embodiments 176-186, wherein W₄ is, for each occurrence, independently selected from C(R₁₂)(R₁₃) and

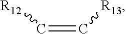

wherein m is an integer selected from 0 to 3, and R₁₂ and R₁₃ are independently selected from hydrogen, deuterium, and fluoro for each occurrence.
Embodiment 188: The method according to any one of Embodiments 176-187, wherein R₁₅ is selected from hydrogen, fluoro, and deuterium.
Embodiment 189: The method according to any one of Embodiments 176-187, wherein R₅ is fluoro, R₄ is hydrogen, R₆ is hydrogen, and R₇ is hydrogen; R₃ and R_{3′} are each hydrogen; X and Y are each hydrogen; and W₄ is, for each occurrence, independently selected from C(R₁₂)(R₁₃) and

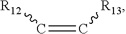

wherein m is an integer selected from 0 to 3, and R₁₂ and R₁₃ are independently selected from hydrogen, deuterium, and fluoro for each occurrence; and R₁₅ is selected from hydrogen, fluoro and deuterium.
Embodiment 190: The method according to Embodiment 189, wherein m is an integer selected from 1 and 2, and R₁₅ is selected from hydrogen and fluoro.
Embodiment 191: The method according to Embodiment 190, wherein W₁ is selected from S and Se.
Embodiment 192: The method according to Embodiment 190, wherein W₁ is O.
Embodiment 193: The method according to any one of Embodiments 176-192, wherein W₄ is C(R₁₂)(R₁₃) and m is 2.
Embodiment 194: The method according to any one of Embodiments 176-193, wherein R₅ is fluoro and R₄, R₆ and R₇ are each hydrogen.
Embodiment 195: The method according to any one of Embodiments 176-194, wherein R₁₂ and R₁₃, independently for each occurrence, are selected from hydrogen and deuterium Embodiment 196: The method according to Embodiment 195, wherein R₃ and R_{3′} are deuterium.
Embodiment 197: The method according to Embodiment 195, wherein R₃ and R_{3′} are each hydrogen.
Embodiment 198: The method according to any one of Embodiments 176-197, wherein the subject is orally administered the compound in an amount of about 0.25 mg/kg to about 2.50 mg/kg once or twice per day.
Embodiment 199: The method according to any one of Embodiments 176-197, wherein the subject is orally administered the compound in an amount of about 0.25 mg/kg to about 2.50 mg/kg once per day.
Embodiment 200: The method according to any one of Embodiments 176-199, wherein the subject is at least 18 years old.
Embodiment 201: The method according to any one of Embodiments 176-200, wherein the subject is administered from about 40 mg to about 80 mg of the compound once per day.
Embodiment 202: The method according to any one of Embodiments 176-201, wherein the compound is administered in a solid capsule or solid tablet.
Embodiment 203: The method according to any one of Embodiments 176-200, wherein the subject is administered about 40 mg to about 80 mg of the compound twice per day.
Embodiment 204: The method according to any one of Embodiment 203, wherein the compound is administered in a solid capsule or solid tablet.
Embodiment 205: The method according to Embodiment 176, wherein the at least one compound is selected from:

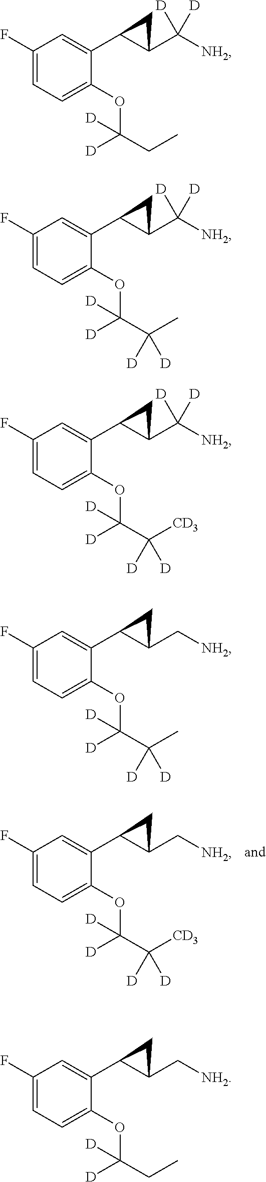

Embodiment 206: A method of treating Prader-Willi Syndrome and/or at least one symptom associated therewith, comprising orally administering to a subject in need thereof from about 0.10 mg/kg to about 5.00 mg/kg of a compound of Formula II:

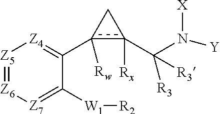

wherein
X and Y are each hydrogen;
W₁ is selected from O, S, S(O), SO₂, Se, Se(O), and SeO₂;
Z₄ is selected from N and CR₄;
Z₅ is selected from N and CR₅;
Z₆ is selected from N and CR₅;
Z₇ is selected from N and CR₇;
R₂, R₄, R₅, R₆ and R₇ are each independently selected from hydrogen, deuterium, halo, —N(R₉)₂, —SR₉, optionally substituted C₁-C₈ alkyl, —C₁-C₈ alkoxy, and optionally substituted C₂-C₈ alkenyl;
R₃, R_{3′}, Rₓ and R_{w}, are each, independently, selected from hydrogen, deuterium, —N(R₉)₂, —SR₉, halo, optionally substituted C₁-C₈ alkyl, —C₁-C₈ alkoxy, optionally substituted C₂-C₈ alkenyl, optionally substituted C₂-C₈ alkynyl, optionally substituted aryl, and optionally substituted heteroaryl;
R₉ is independently selected from hydrogen, deuterium, optionally substituted C₁-C₈alkyl, optionally substituted C₂-C₈ alkenyl, and optionally substituted aryl;
[symbol] is a single bond;
and salts, solvates, hydrates, prodrugs, and enantiomers thereof.
   Embodiment 207: The method according to Embodiment 206, wherein
Z₄ is CR₄;
Z₅ is CR₅;
Z₆ is CR₆;
Z₇ is CR₇; and
R₄, R₅, R₆ and R₇ are each independently selected from hydrogen, deuterium, —N(R₉)₂, —SR₉, halo, optionally substituted C₁-C₈ alkyl, —C₁-C₈ alkoxy, and optionally substituted C₂-C₈ alkenyl.
   Embodiment 208: The method according to any one of Embodiments 206-207, wherein R₄, R₅, R₆, and R₇ are selected from hydrogen, deuterium, and halo.
   Embodiment 209: The method according to any one of Embodiments 206-208, wherein R₅ is fluoro.
   Embodiment 210: The method according to any one of Embodiments 206-209, wherein R₄, R₆, and R₇ are each hydrogen.
   Embodiment 211: The method according to any one of Embodiments 206-210, wherein R₂ is C₁-C₄ alkyl substituted with at least two deuteriums.
   Embodiment 212: The method according to any one of Embodiments 206-211, wherein R₃ and R_{3′} are each independently selected from hydrogen and deuterium.
   Embodiment 213: The method according to any one of Embodiments 206-212, wherein R₂ is optionally substituted C₁-C₄ alkyl.
   Embodiment 214: The method according to Embodiment 213, wherein R₂ is C₁-C₄ alkyl substituted with at least one group selected from cycloalkyl, aryl, and heteroaryl.
   Embodiment 215: The method according to Embodiment 214, wherein the C₁-C₄ alkyl is further optionally substituted with at least one deuterium or fluoro.
   Embodiment 216: The method according to any one of Embodiments 206-213, wherein R₂ is C₁-C₄ alkyl substituted with one or more deuteriums.
   Embodiment 217: The method according to any one of Embodiments 206-212, wherein R₂ is selected from

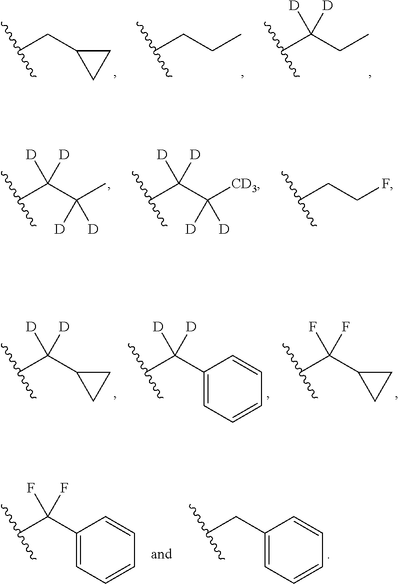

Embodiment 218: The method according to any one of Embodiments 206-216, wherein R₂ is substituted with at least one deuterium.
Embodiment 219: The method according to Embodiment 218, wherein R₂ is substituted with at least two deuteriums.
Embodiment 220: The method according to any one of Embodiments 206-219, wherein R₂ is optionally substituted with at least one halo.
Embodiment 221: The method according to Embodiment 220, wherein R₂ is substituted with fluoro.
Embodiment 222: The method according to any one of Embodiments 206-221, wherein W₁ is selected from S and Se.
Embodiment 223: The method according to any one of Embodiments 206-221, wherein W₁ is 0.
Embodiment 224: The method according to Embodiment 223, wherein
R₂ is selected from

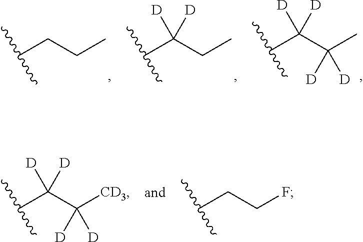

R₅ is fluoro;
R₄, R₆, and R₇ are each hydrogen;
R₃ and R_{3′} are each independently selected from hydrogen and deuterium; and
Rₓ and R_{w} are each hydrogen.
   Embodiment 225: The method according to any one of Embodiments 206-224, wherein the subject is orally administered the at least one compound in an amount of from about 0.25 mg/kg to about 2.50 mg/kg once or twice per day.
   Embodiment 226: The method according to any one of Embodiments 206-224, wherein the subject is orally administered the at least one compound in an amount of from about 0.25 mg/kg to about 2.50 mg/kg once per day.
   Embodiment 227: The method according to any one of Embodiments 206-226, wherein the subject is at least 18 years old.
   Embodiment 228: The method according to any one of Embodiments 206-227, wherein the at least one compound is administered with food.
   Embodiment 229: The method according to any one of Embodiments 206-228, wherein the subject is administered from about 40 mg to about 80 mg of the at least one compound once per day.
   Embodiment 230: The method according to any one of Embodiments 206-229, wherein the at least one compound is administered in a solid capsule or solid tablet.
   Embodiment 231: The method according to any one of Embodiments 206-228, wherein the subject is administered from about 40 mg to about 80 mg of the at least one compound twice per day.
   Embodiment 232: The method according to any one of Embodiments 206-228 and 231, wherein the at least one compound is administered in a solid capsule or solid tablet.
   Embodiment 233: A method of treating a disease or disorder, comprising orally administering to a subject in need thereof a therapeutically effective amount at least one compound, wherein the at least one compound is selected from compounds of Formula V:

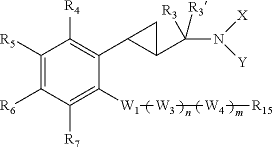

wherein
X and Y are each independently selected from hydrogen, deuterium, optionally substituted C₁-C₈ alkyl, optionally substituted C₂-C₈ alkenyl, and optionally substituted C₂-C₈ alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from CF₂, NR₁, O, S, S(O), SO₂, Se, Se(O), and SeO₂;
W₃ is, for each occurrence, independently selected from optionally substituted alkylene, optionally substituted arylene, optionally substituted heteroarylene, optionally substituted cycloalkylene, optionally substituted heterocycloalkylene, and

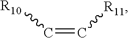

wherein n is an integer selected from 1 to 10;
W₄ is, for each occurrence, independently selected from O, S, Se, C(R₁₂)(R₁₃), optionally substituted arylene, optionally substituted heteroarylene, optionally substituted cycloalkylene, optionally substituted heterocycloalkylene, and

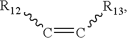

wherein m is an integer selected from 0 to 10;
R₁₀, R₁₁, R₁₂, R₁₃ and R₁₅ are, for each occurrence, independently selected from C₁-C₈ alkyl, hydrogen, deuterium, halo, and hydroxyl;
R₁ is selected from hydrogen, deuterium, optionally substituted C₁-C₈ alkyl, optionally substituted C₂-C₈ alkenyl, —C(O)R₈, —C(O)OR₈, —P(O)(OR₉)₂, —C(O)N(R₉)₂, —SOR₈, and —SO₂R₈;
R₄, R₅, R₆, and R₇ are each independently selected from hydrogen, deuterium, —N(R₉)₂, —SR₉, halo, optionally substituted C₁-C₈ alkyl, —C₁-C₈ alkoxy, and optionally substituted C₂-C₈ alkenyl, or R₄ is taken together with R₅ and the carbons therebetween to form 3- to 7-membered carbocyclic or heterocyclic ring optionally including 1 to 2 additional ring heteromoieties selected from O, S, S(O), SO₂, and NR₅, or R₅ is taken together with R₆ and the carbons therebetween to form 3- to 7-membered carbocyclic or heterocyclic ring optionally including 1 to 2 additional ring heteromoieties selected from O, S, S(O), SO₂, and NR₉, or R₆ is taken together with R₇ and the carbons therebetween to form 3- to 7-membered carbocyclic or heterocyclic ring optionally including 1 to 2 additional ring heteromoieties selected from O, S, S(O), SO₂, and NR₉;
R₃ and R_{3′} are each, independently, selected from hydrogen, deuterium, —N(R₉)₂, —SR₅, halo, optionally substituted C₁-C₈ alkyl, —C₁-C₈ alkoxy, and optionally substituted C₂-C₈ alkenyl;
R₈ is selected from optionally substituted C₁-C₈ alkyl, optionally substituted C₂-C₈ alkenyl, and optionally substituted aryl;
R₉ is independently selected from hydrogen, deuterium, optionally substituted C₁-C₈alkyl, optionally substituted C₂-C₈ alkenyl, and optionally substituted aryl;
and salts, solvates, hydrates, prodrugs, and enantiomers thereof.
   Embodiment 234: The method according to Embodiment 233, wherein X and Y are each hydrogen.
   Embodiment 235: The method according to any one of Embodiments 233-234, wherein W₁ is selected from O, S, and Se.
   Embodiment 236: The method according to any one of Embodiments 233-235, wherein R₄, R₅, R₆, and R₇ are each independently selected from hydrogen, deuterium, and halo.
   Embodiment 237: The method according to any one of Embodiments 233-236, wherein R₃ and R_{3′} are each, independently, selected from hydrogen and deuterium.
   Embodiment 238: The method according to any one of Embodiments 233-237, wherein the at least one compound of Formula V is selected from compounds of Formula VI(a):

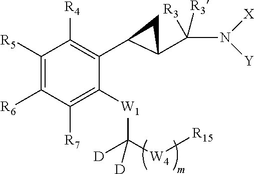

and salts, solvates, hydrates, prodrugs, and enantiomers thereof.
   Embodiment 239: The method according to any one of Embodiments 233-238, wherein R₄, R₅, R₆, and R₇ are each independently selected from hydrogen, deuterium, and fluoro.
   Embodiment 240: The method according to Embodiment 239, wherein R₅ is fluoro.
   Embodiment 241: The method according to Embodiment 240, wherein W₁ is selected from S and Se.
   Embodiment 242: The method according to Embodiment 240, wherein W₁ is O.
   Embodiment 243: The method according to any one of Embodiments 233-242, wherein R₃ and R_{3′} are each hydrogen.
   Embodiment 244: The method according to any one of Embodiments 233-243, wherein R₄, R₆, and R₇ are each hydrogen.
   Embodiment 245: The method according to Embodiment 244, wherein W₁ is selected from S and Se.
   Embodiment 246: The method according to Embodiment 244, wherein W₁ is O.
   Embodiment 247: The method according to any one of Embodiments 233-246, wherein W₄ is, for each occurrence, independently selected from C(R₁₂)(R₁₃) and

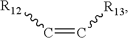

wherein m is an integer selected from 0 to 3, and R₁₂ and R₁₃ are each independently selected from hydrogen, deuterium, and fluoro for each occurrence.
Embodiment 248: The method according to any one of Embodiments 233-247, wherein R₁₅ is selected from hydrogen, fluoro, and deuterium.
Embodiment 249: The method according to Embodiment 233, wherein R₅ is fluoro, R₄ is hydrogen, R₆ is hydrogen, and R₇ is hydrogen; R₃ and R_{3′} are each hydrogen; and W₄ is, for each occurrence, independently selected from C(R₁₂)(R₁₃) and

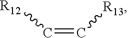

wherein m is an integer selected from 0 to 3, and R₁₂ and R₁₃ are each independently selected from hydrogen, deuterium, and fluoro for each occurrence; and R₁₅ is selected from hydrogen, fluoro, and deuterium.
Embodiment 250: The method according to Embodiment 249, wherein m is an integer selected from 1 and 2, and R₁₅ is selected from hydrogen and fluoro.
Embodiment 251: The method according to any one of Embodiments 249-250, wherein W₁ is selected from S and Se.
Embodiment 252: The method according to any one of Embodiments 249-250, wherein W₁ is 0.
Embodiment 253: The method according to any one of Embodiments 233-252, wherein the disease or disorder is Prader-Willi Syndrome (PWS).
Embodiment 254: The method according to claim 253, wherein the disease or disorder is one or more symptoms associated with Prader-Willi Syndrome (PWS).
Embodiment 255: The method according to any one of Embodiments 233-254, wherein the subject is an adult subject that is aged at least 18 years.
Embodiment 256: The method according to any one of Embodiments 233-255, wherein the at least one compound is orally administered.
Embodiment 257: The method according to any one of Embodiments 233-256, wherein the at least one compound is administered in an amount of about 0.10 mg/kg to about 5.00 mg/kg.
Embodiment 258: The method according to any one of Embodiments 233-257, wherein the subject is orally administered the at least one compound in an amount of about 0.10 mg/kg to about 5.00 mg/kg once or twice per day.
Embodiment 259: The method according to any one of Embodiments 233-257, wherein the subject is orally administered the at least one compound in an amount of about 0.10 mg/kg to about 2.50 mg/kg once per day.
Embodiment 260: The method according to any one of Embodiments 233-259, wherein the subject is administered about 10 mg to about 500 mg of the at least one compound once per day.
Embodiment 261: The method according to any one of Embodiments 233-259, wherein the subject is administered about 10 mg to about 500 mg of the at least one compound twice per day.
Embodiment 262: The method according to any one of Embodiments 233-261, wherein the disease or disorder is associated with food intake.
Embodiment 263: The method according to any one of Embodiments 233-252 or 255-262, wherein the disease or disorder is a seizure disease or disorder, and/or symptoms associated therewith.
Embodiment 264: The method according to any one Embodiments 233-252 or 255-263, wherein the disease or disorder is staring spells and/or loss of awareness associated with a thalamocortical circuit disruption and/or thalamocortical oscillations.
Embodiment 265: The method according to Embodiments 264, wherein the staring spells and/or loss of awareness are associated with eyelid myoclonia.
Embodiment 266: The method according to any one of Embodiments 233-265, wherein the at least one compound is crystalline.
Embodiment 267: The method according to any one of Embodiments 233-266, wherein the at least one compound is a crystalline salt.
Embodiment 268: The method according to any one of Embodiments 264-267, wherein the thalamocortical oscillations are associated with absence seizures.
Embodiment 269: The method according to any one of Embodiments 264-268, wherein the thalamocortical oscillations associated with non-convulsive seizures.
Embodiment 270: The method according to any one of Embodiments 233-252 or 255-269, wherein the disease or disorder is absence seizures.
Embodiment 271: The method according to any one of Embodiments 233-252 or 255-269, wherein the disease or disorder is Developmental Epileptic Encephalopathy.
Embodiment 272: The method according to any one of Embodiments 233-252 or 255-269, wherein the disease or disorder is at least one symptom associated with Developmental Epileptic Encephalopathy.
Embodiment 273: The method according to any one of Embodiments 233-272, wherein the at least one compound is administered in a liquid oral dosage form comprising sucrose and water.
Embodiment 274: The method according to any one of Embodiments 233-273, wherein the at least one compound is administered at least two times per day.
Embodiment 275: The method according to any one of Embodiments 233-273, wherein the at least one compound is administered one to three times per day.
Embodiment 276: The method according to any one of Embodiments 233-273, wherein the at least one compound is administered once per day.
Embodiment 277: The method according to any one of Embodiments 233-272 or 274-276, wherein the at least one compound is administered in an extended release tablet further comprising a pharmaceutically-acceptable carrier.
Embodiment 278: The method according to Embodiments 277, wherein the extended release tablet delivers at least some of the at least one compound to the small intestine of the subject.
Embodiment 279: The method according to any one of Embodiments 277-278, wherein the extended release tablet releases the at least one compound over a time period of from about 8 hrs to about 36 hrs.
Embodiment 280: The method according to any one of Embodiments 277-278, wherein the extended release tablet releases the at least one compound over a time period of from about 12 hrs to about 24 hrs.
Embodiment 281: The method according to any one of Embodiments 277-280, wherein the at least one compound is administered once per day.
Embodiment 282: The method according to any one of Embodiments 98-281, wherein the at least one compound induces a G_{q}-mediated signaling bias at 5-HT_{2C} receptors when compared to β-arrestin-mediated signaling.
Embodiment 283: The method according to any one of Embodiments 233-282, wherein the at least one compound is a 5-HT_{2C} receptor agonist.
Embodiment 284: The method according to Embodiments 270 and 273-283, wherein the absence seizures are associated with Developmental Epileptic Encephalopathy.
Embodiment 285: The method according to Embodiment 284, wherein the absence seizures are atypical.
Embodiment 286: The method according to any one of Embodiments 284-285, wherein the subject exhibits EEG spike-and-wave patterns of about 2.5 Hz or less.
Embodiment 287: The method according to any one of Embodiments 270 and 273-283, wherein the absence seizures are typical.
Embodiment 288: The method according to Embodiment 287, wherein the subject exhibits EEG spike-and-wave patterns of about 3.0 Hz or more.
Embodiment 289: The method according to any one of Embodiments 264-265, wherein the wherein the staring spells occur for from about 5 seconds to about 90 seconds, such as about 3 to about 15 seconds.
Embodiment 290: The method according to any one of Embodiments 233-252, 255-267, or 273-283, wherein the disease or disorder is obesity.
Embodiment 291: The method according to Embodiment 290, wherein the disease or disorder is obesity and at least one comorbidity.
Embodiment 292: The method according to Embodiment 291, wherein the at least one comorbidity is selected from hypertension, Type II diabetes, dyslipidemia, stroke, gout, osteoarthritis, and sleep apnea.
Embodiment 293: The method according to any one of Embodiments 290-292, wherein the subject has a body mass index (BMI) of about 27 kg/m² or more.
Embodiment 294: The method according to any one of Embodiments 290-292, wherein the subject has a body mass index (BMI) of about 30 kg/m² or more.
Embodiment 295: The method according to any one of Embodiments 290-294, further comprising administering to the subject at least one GLP-1 receptor agonist.
Embodiment 296: The method according to Embodiment 295, wherein the at least one GLP-1 receptor agonist is selected from semaglutide, liraglutide, tirzepatide, exenatide, albiglutide, dulaglutide, and lixisenatide.
Embodiment 297: The method according to any one of Embodiments 290-296, wherein the at least one compound is selected from Lumocaserin, deuterated Lumocaserin, and salts, solvates, hydrates, or prodrugs thereof.
Embodiment 298: The method according to Embodiment 297, wherein the at least one compound is selected from:

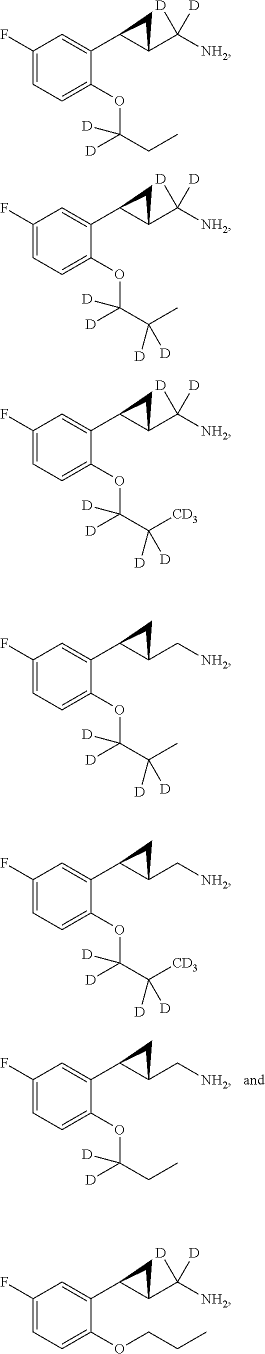

and salts, solvates, hydrates, or prodrugs thereof.
Embodiment 299: A method of treating a metabolic disorder, comprising administering to a subject:
at least one 5-HT_{2C} receptor agonist; and
a GLP-1 receptor agonist,
   wherein the 5-HT_{2C} receptor agonist is administered at least once every other day, and the GLP-1 receptor agonist is administered at least once per month.
      Embodiment 300: The method of Embodiment 299, wherein the at least one 5-HT_{2C} receptor agonist is functionally biased.
      Embodiment 301: The method according to any one of Embodiments 299-300, wherein the 5-HT_{2C} receptor agonist is selected from compounds of Embodiments 1-89.
      Embodiment 302: The method according to any one of Embodiments 299-301, wherein the at least one 5-HT_{2C} receptor agonist is administered orally.
      Embodiment 303: The method according to any one of Embodiments 299-302, wherein the GLP-1 agonist is administered by injection.
      Embodiment 304: A method of treating a metabolic disorder, comprising providing instructions to a patient or a healthcare provider to:
administer at least one 5-HT_{2C} receptor agonist; and
administer a GLP-1 receptor agonist,
   wherein the instructions indicate that the combination therapy improves weight loss outcomes relative to monotherapy.
      Embodiment 305: A method of inducing weight loss in an overweight or obese subject comprising:
administering a 5-HT_{2C} receptor agonist having a half-life of at least 5 hours; and
administering a GLP-1 receptor agonist having a half-life of at least 72 hours,
   wherein the subject achieves at least a 5% reduction in body weight after 12 weeks of therapy.
      Embodiment 306: A method of treating obesity comprising:
initiating treatment with a GLP-1 receptor agonist administered at least once per month;
identifying a subject with suboptimal weight loss after 4-8 weeks of GLP-1 receptor agonist monotherapy; and
initiating co-treatment with a selective 5-HT_{2C} receptor agonist at least once every other day,
   optionally wherein the co-treatment results in a statistically significant increase in mean weight loss compared to continued GLP-1 receptor agonist monotherapy.
      Embodiment 307: A method of treating a metabolic disorder in a subject, comprising the simultaneous, sequential, or separate administration of:
a 5-HT_{2C} receptor agonist; and
a GLP-1 receptor agonist.
   Embodiment 308: A method of treating a metabolic disorder comprising:
selecting a patient that has previously been administered a GLP-1 receptor agonist treatment regimen for treating the metabolic disorder, wherein the treatment regimen did not achieve a desired outcome; and
administering to the patient a combination of a GLP-1 receptor agonist and a 5-HT_{2C} receptor agonist, wherein the administration of the combination is conducted simultaneously, sequentially or separately, and said administration achieves the desired outcome.
   Embodiment 309: A kit for treating obesity comprising:
a first container comprising an oral dosage form of a 5-HT_{2C} receptor agonist;
a second container comprising an injectable dosage form of a GLP-1 receptor agonist; and
instructions (such as printed instructions) for concomitant use of the 5-HT_{2C} receptor agonist and the GLP-1 receptor agonist for the treatment of obesity.
   Embodiment 310: The kit of Embodiment 309, wherein the instructions include language printed on paper or another medium within the kit.
   Embodiment 311: The kit according to any one of Embodiments 309-310, wherein the instructions are accessed by a barcode or QR code contained in the kit, wherein scanning of said code with a smartphone or other device provides the instructions in digital form.
   Embodiment 312: A method of treating binge eating disorder in a human subject comprising concomitantly administering:
a therapeutically effective amount of a 5-HT_{2C} receptor agonist at least once every other day; and
a therapeutically effective amount of a GLP-1 receptor agonist at least once per month,
   optionally wherein the treatment results in a reduction in binge eating episodes and/or overall weight loss.
      Embodiment 313: A system for promoting weight loss in a subject comprising:
a regimen of administration of a 5-HT_{2C} receptor agonist and a GLP-1 receptor agonist; and
a software application configured to track at least one of medication adherence, monitor weight and appetite changes, and/or to adjust dosage recommendations based on subject response.
   Embodiment 314: A method of treating obesity in a human subject comprising:
(a) administering to the subject a 5-HT_{2C} receptor agonist at least once every other day; and
(b) administering to the subject a glucagon-like peptide-1 (GLP-1) receptor agonist at least once per month,
   optionally wherein the combination of (a) and (b) provides for a synergistic effect characterized by a reduction in body weight that is greater than the sum of the reductions observed when each agonist is administered as a monotherapy.
      Embodiment 315: A method of increasing brain stem levels of 5-hydroxytryptamine (serotonin) and/or 5-hydroxyindoleacetic acid (5-HIAA), comprising administering to a subject in need thereof a therapeutically effective amount of a functionally biased 5-HT_{2C} receptor agonist.
      Embodiment 316: A method of preventing or ameliorating sudden unexpected death in epilepsy (SUDEP), comprising administering to a subject in need thereof a therapeutically effective amount of at least one 5-HT_{2C} receptor agonist.
      Embodiment 317: The method or kit according to any one of Embodiments 302-316, wherein the 5-HT_{2C} agonist is functionally biased.
      Embodiment 318: The method or kit according to any one of Embodiment 302-317, wherein 5-HT_{2C} receptor agonist is selected from compounds of Embodiments 1-89.
      Embodiment 319: The compound according to any one of Embodiments 1-89, wherein the at least one compounds is a hydrate.
      Embodiment 320: The compound according to Embodiment 319, wherein the at least one compounds is a non-stoichiometric hydrate.
      Embodiment 321: The compound according to any one of Embodiments 319-320, wherein the at least one compounds is pore hydrate.
      Embodiment 322: The compound according to any one of Embodiments 319-320, wherein the at least one compounds is channel hydrate.
      Embodiment 323: The compound according to any one of Embodiments 319-322, wherein the at least one compounds is salt.
      Embodiment 324: The compound according to Embodiment 323, wherein the at least one compounds is an HCl salt.

Finally, it is noted that, as used in this specification and the appended claims, the singular forms “a,” “an,” and “the,” include plural referents unless expressly and unequivocally limited to one referent and vice versa. As used herein, the term “include” or “comprising” and its grammatical variants are intended to be non-limiting, such that recitation of an item or items is not to the exclusion of other like items that can be substituted or added to the recited item(s).

1.-15. (canceled)

16. A method of treating at least one symptom of Prader-Willi Syndrome, comprising administering to a subject having Prader-Willi Syndrome a therapeutically effective amount of at least one compound of Formula I: 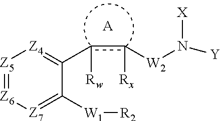
wherein
X and Y are each hydrogen;
W₁ is O;
W₂ is C(R₃)(R_{3′});
Z₄ is CR₄;
Z₅ is CR₅;
Z₆ is CR₆;
Z₇ is CR₇;
R₂ is a C₁-C₄ alkyl optionally substituted with one or more groups selected from deuterium and halo;
R₃ and R_{3′} are each, independently, selected from hydrogen and deuterium;
R₄, R₆, and R₇ are each independently selected from hydrogen and deuterium;
R₅ is fluoro;
Group A is cyclopropyl, wherein the [symbol] is a single bond and wherein Rₓ and R_{w} are each, independently, selected from hydrogen and deuterium;
and salts, solvates, hydrates, prodrugs, and enantiomers thereof.

17. The method of claim 16, wherein R₂ is a C₁-C₄ alkyl optionally substituted with at least one deuterium or fluoro.

18. The method of claim 16, wherein R₂ is a C₁-C₄ alkyl substituted with at least one deuterium.

19. The method of claim 16, wherein R₂ is a C₁-C₄ alkyl substituted with at least one fluoro.

20. The method of claim 16, wherein R₃ and R_{3′} are each deuterium.

21. The method of claim 16, wherein R₂ is selected from methyl, ethyl and propyl, each of which is optionally substituted with one or more groups selected from deuterium and halo.

22. The method of claim 16, wherein R₂ is n-propyl optionally substituted with one or more groups selected from deuterium and halo.

23. The method of claim 22, wherein R₂ is unsubstituted.

24. The method of claim 22, wherein R₂ is substituted with at least one deuterium.

25. The method of claim 16, wherein the at least one compound is 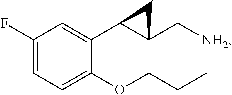 [SMILES: CCCOc1ccc(F)cc1[C@H]1C[C@@H]1CN]
or a salt, solvate, hydrate, or prodrug thereof.

26. The method of claim 25, wherein the at least one compound comprises an HCl salt.

27. The method of claim 26, wherein the HCl salt comprises a non-stoichiometric hydrate.

28. The method of claim 16, wherein the at least one compound is 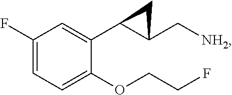 [SMILES: NC[C@H]1C[C@@H]1c1cc(F)ccc1OCCF]
or a salt, solvate, hydrate, or prodrug thereof

29. The method of claim 28, wherein the at least one compound comprises an HCl salt.

30. The method of claim 29, wherein the HCl salt comprises a non-stoichiometric hydrate.

31. The method of claim 16, wherein the at least one compound is selected from: 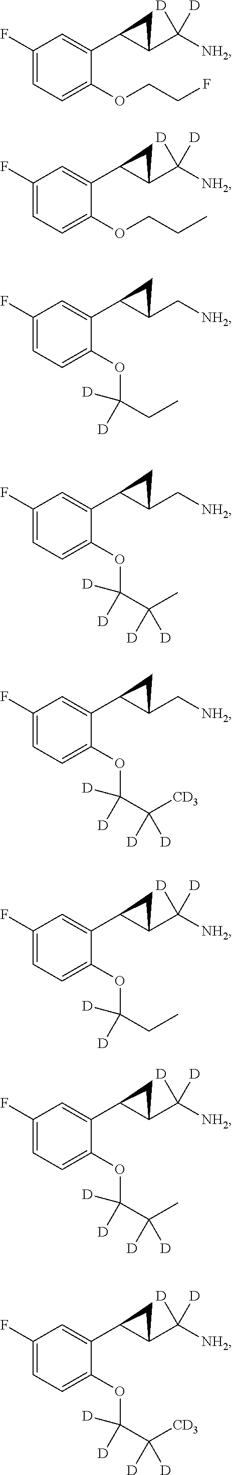
and salts, solvates, hydrates, and prodrugs thereof.

32. The method of claim 16, wherein the subject is a human subject.

33. The method of claim 32, wherein the at least one compound is administered orally.

34. The method of claim 32, wherein the subject is an adult subject 18 years of age or older.

35. The method of claim 16, wherein the subject is administered the at least one compound in an amount of from about 0.25 mg/kg to about 2.50 mg/kg one to three times per day.

36. The method of claim 16, wherein the at least one symptom of Prader-Willi Syndrome is selected from a compulsive behavior, a disturbance in REM sleep, anxiety, or hyperphagia.

37. The method of claim 36, wherein the at least one symptom comprises hyperphagia.

38. The method of claim 37, wherein administering the at least one compound reduces a constant feeling of hunger in the subject.

39. The method of claim 36, wherein the at least one symptom comprises a compulsive behavior.

40. The method of claim 39 wherein administering the at least one compound reduces alcohol- or drug-seeking behavior in the subject.
